(19) United States
(12) Patent Application Publication
(10) Pub. No.: US 2014/0315188 A1
(43) Pub. Date: Oct. 23, 2014

(54) ASSESSMENT OF PML RISK AND METHODS BASED THEREON

(71) Applicant: WESTFAELISCHE WILHEMS-UNIVERSITAET MUENSTER, Muenster (DE)

(72) Inventors: Nicholas Schwab, Muenster (DE); Tilman Schneider-Hohendorf, Muenster (DE); Heinz Wiendl, Muenster (DE)

(21) Appl. No.: 14/351,782

(22) PCT Filed: Oct. 16, 2012
(86) PCT No.: PCT/EP2012/070472

§ 371 Date: Apr. 14, 2014

(30) Foreign Application Priority Data
Mar. 7, 2012 (EP) 12158369.4

(51) Int. Cl. G01N 33/569

(52) U.S. Cl. CPC G01N 33/56966
USPC 435/5

(57) ABSTRACT

The invention provides a method of assessing the risk of occurrence of progressive multifocal leukoencephalopathy (PML) in a subject as well as a method of stratifying a subject undergoing α₄-integrin and/or VLA-4 blocking agent treatment for suspension of the treatment and a method of stratifying a subject undergoing Highly Active Antiretroviral Therapy (HAART) for alteration of HAART. These methods comprise detecting the level of P-selectin glycoprotein ligand-1 (PSGL-1) expressing T cells in a sample from the subject.

### CROSS-REFERENCE TO RELATED APPLICATIONS

The present application claims the benefit of and the priority to an application for “Risk Stratification of Patients Receiving VLA-4 Blocking Agents” filed on 17 Oct. 2011 with the European Patent Office, and there duly assigned serial number EP 11 185 439. The present application further claims the benefit of and the priority to an application for “Methods of Risk Assessment of PML” filed on 7 Mar. 2012 with the European Patent Office, and there duly assigned serial number EP 12 158 369. The contents of said applications filed on 17 Oct. 2011 and 7 Mar. 2012 are incorporated herein by reference for all purposes in their entirety including all tables, figures, and claims—as well as including an incorporation of any element or part of the description, claims or drawings not contained herein and referred to in Rule 20.5(a) of the PCT, pursuant to Rule 4.18 of the PCT.

### FIELD OF THE INVENTION

The present invention relates to the assessment of progressive multifocal leukoencephalopathy (PML) risk, i.e. assessing the risk of occurrence of PML, in a subject. The invention also relates to methods based on such risk assessment. The invention provides a method of stratifying a subject undergoing Highly Active Antiretroviral Therapy (HAART) for alteration of HAART, as well as a method of stratifying a subject undergoing α₄-integrin blocking agent and/or VLA-4 blocking agent treatment for suspension of this α₄-integrin/VLA-4 blocking agent treatment. Provided are further a method of treating retroviral infection so as to avoid the occurrence of PML and a method of administering an α₄-integrin blocking agent or a VLA-4 blocking agent to a subject so as to avoid the occurrence of PML. The invention further provides a method of treating a subject with an autoimmune disease. The invention also provides a method of treating a subject with a retroviral infection such as HIV.

### BACKGROUND OF THE INVENTION

The following discussion of the background of the invention is merely provided to aid the reader in understanding the invention and is not admitted to describe or constitute prior art to the present invention.

Multifocal leukoencephalopathy (PML) is a neurodegenerative disease that may typically occur in the course of advanced HIV/AIDS. PML is also a potential adverse effect of a certain therapy of multiple sclerosis and Crohn's disease.

Acquired immunodeficiency syndrome (AIDS) is a disease of the human immune system first recognized in the U.S. in 1981. Cases were identified on the basis of severe opportunistic infections, and the disease was later found to be caused by the HIV. As of 2011, the World Health Organisation estimated that there were about 34.2 million people worldwide living with HIV/AIDS.

The development of new antiretroviral agents including nucleoside reverse transcriptase inhibitors, nonnucleoside reverse transcriptase inhibitors and protease inhibitors has changed HIV/AIDS from an acute/subacute fatal infection to a chronic disease. The development of antiretroviral therapies has also had a significant impact on the neurological manifestations of HIV. The prolonged survival of patients with HIV/AIDS on Highly Active Antiretroviral Therapy (HAART) has shifted the prevalence of HIV-related neurological diseases to older age groups and is creating a population that is at risk for developing neurodegenerative diseases of later life.

Multiple sclerosis (MS) is a chronic, inflammatory central nervous system (CNS) disease, characterized pathologically by demyelination. MS has also been classified as an autoimmune disease. MS disease activity can be monitored by cranial scans, including magnetic resonance imaging (MRI) of the brain, accumulation of disability, as well as rate and severity of relapses. There are five distinct disease stages and/or types of MS, namely, (1) benign multiple sclerosis; (2) relapsing-remitting multiple sclerosis; (3) secondary progressive multiple sclerosis; (4) progressive relapsing multiple sclerosis; and (5) primary progressive multiple sclerosis.

Crohn's disease is a type of inflammatory bowel disease. It typically manifests in the gastrointestinal tract and can be categorized by the specific tract region affected. It is thought to be an autoimmune disease, in which the body's immune system attacks the gastrointestinal tract, causing inflammation of the gastrointestinal tract. The disease manifestations usually are isolated to the digestive tract, but other manifestations such as inflammation of skin structures, the eyes, and the joints have been well described. The disease is known to have spontaneous exacerbations and remissions. Unfortunately, the cause of Crohn's disease is not known, and there is no known cure for Crohn's disease.

Crohn's disease has an immune response pattern that includes an increased production of interleukin-12, tumour necrosis factor (TNF) and interferon-γ. Tumor necrosis factor (TNF) has been identified as an important cytokine in the pathogenesis of Crohn's disease, with elevated concentrations playing a role in pathologic inflammation. The increased production of TNF by macrophages in patients with Crohn's disease results in elevated concentrations of TNF in the stool, blood, and mucosa. In recent years, biologic response modifiers that inhibit TNF activity have become potential therapies for treating Crohn's disease.

The humanized monoclonal immunoglobulin Natalizumab, directed against the α4-subunit of α4β1 (VLA-4, Very Late Antigen-4) and α4β7 (LPAM-1, Lymphocyte Peyer's Patch Adhesion Molecule 1) integrins expressed on the surface of activated lymphocytes, has been used in the treatment of both MS and Crohn's disease. Natalizumab is both clinically effective and generally well-tolerated. However, Natalizumab treatment for longer than 18 months has been found to be associated with an enhanced risk of developing PML. PML has almost exclusively been found in immunocompromised individuals, especially in subjects with reduced cellular immunity. It has also been reported in rheumatic diseases. PML has for example been found in individuals with hematological malignancies and lymphoproliferative diseases, individuals with Hodgkin's lymphoma, individuals with systemic lupus erythematosus or subjects receiving immunosuppressive medication such as transplant patients. In addition to Natalizumab therapy, PML has also been found to be associated with therapy using the monoclonal antibodies Rituximab, used in the treatment of lymphomas, leukemias, transplant rejection and certain autoimmune disorders, and Efalizumab, formerly used in the treatment of autoimmune diseases, in particular psoriasis. In view of the risk of PML, Efalizumab has currently been withdrawn from the U.S. market. Natalizumab, first approved in 2004 by the U.S. Food and Drug Administration (FDA) for the treatment of multiple sclerosis, was withdrawn from the market after it was linked with three cases of PML. After a review of safety information and no additional cases of PML were identified in previously treated patients the antibody was re-introduced in the U.S. and approved in the European Union in July 2006. Natalizumab has now been restricted as a monotherapy for adult relapsing remitting multiple sclerosis (RRMS) patients with high disease activity. Natalizumab is also still approved as a monotherapy for adults with moderate-to-severe active Crohn's disease.

PML is caused by lytic infection of oligodendrocytes by the John Cunningham virus (JCV), a double-stranded, not enveloped human polyomavirus. So far there have been over 150 cases of JCV-induced PML associated with the treatment of MS patients with Natalizumab with a mortality rate of so far 20%. It is still largely unknown how the treatment with blocking integrins α₄β₁/VLA-4 and/or α₄β₇/LPAM-1 interferes with JCV control or immune surveillance (Tan, C. S, and Koralnik, I. J., Lancet Neurol. (2010) 9, 4, 425-437). The majority of PML cases is, nevertheless, represented by individuals infected with HIV (supra). While the availability of potent antiretroviral therapies has led to a decrease in the incidence of PML, HIV/AIDS-associated PML morbidity and mortality remain high (Hernandez, B., et al., Expert Opin. Pharmacother. [2009] 10, 3, 403-416). JCV is difficult to study as it grows only in a few cell types in vitro (human fetal glial cells or adult glioma or neuroblastoma cell lines) and no animal models exist.

Prognosis of PML is poor, since no specific therapy is available. While only 20% of the Natalizumab-treated PML patients so far died, the overall mortality of PML has been reported to be above 50%. In the absence of any therapy it would be particularly helpful to be able to predict the risk whether an individual, in particular an individual suffering from HIV infection, is likely to develop PML. Hence, there exists a need for means to determine at an early stage, i.e. before the onset of the disease, whether an HIV positive individual is likely to suffer from PML.

Recent studies suggest that patients under treatment with the α₄-integrin-blocking agent Natalizumab for more than 12 months are at elevated risk for PML, with the risk increasing after approximately 18 months of treatment, and can reach risk levels of up to 1:120. It is not known if the risk of developing PML continues to increase, remains the same, or decreases after a patient has been on Natalizumab for more than three years. Since there is a clear risk association between Natalizumab and the development of PML after long-term treatment of the α₄-integrin-blocking agent Natalizumab, there is an urgent need to identify those patients who are more prone to PML. However, only few candidates as indicators in this regard have evolved: (1) treatment duration, (2) pre-treatment with immunosuppressive drugs, and (3) presence of JCV antibodies in serum.

European patent application EP 2 226 392 A1 discloses an immunological method for detecting an extra renal active infection by JCV in a patient who is a candidate for immunosuppressive treatment. The method of EP 2 226 392 A1 includes screening for the presence of activated T lymphocytes against JCV.

U.S patent application 2010/0196318 discloses testing for serum anti-JCV antibody prior to initiating Natalizumab therapy in patients. However, the detection of JCV antibody in an individual does not predict the risk for PML and therefore cannot advise a medical professional whether or not to continue the treatment. U.S. patent application 2009/0216107 discloses a method of screening patients undergoing Natalizumab treatment by testing the patient's cerebrospinal fluid to detect the presence of cytomegalovirus, JCV, Toxoplasma gondii, Epstein-Barr virus, Cryptococcus neoformans and tuberculosis by PCR, as well as examining the retinal status to detect the presence of ocular cytomegalovirus. If an indication of the presence of the virus is detected, Natalizumab treatment should be discontinued. However, such methods are only precautionary measures which also do not indicate a risk of developing PML. There still remains a need to develop a method to determine the risk of a subject to develop PML who receive an α₄-integrin-blocking agent on an individual basis. It would be advantageous if the determination could help the practitioner to identify patients who are particularly prone to PML or stop the treatment in time before the immune competence of the subject deteriorates.

It is therefore an object of the present invention to provide a method that is suitable for determining the risk for PML development in a subject. It would be advantageous if such method can be used to monitor the immune competence of patients receiving or expected to receive Natalizumab thus to avoid the possible development of PML or even another complication at a later stage. It is a further object of the invention to provide a method for assessing the likelihood of PML occurrence in a subject suffering from HIV. It is yet a further object of the invention to provide a therapeutic method or use for a subject under HAART or under treatment with an α₄-integrin-blocking agent that avoids the occurrence of PML. These objects are solved by the methods of the independent claims.

### SUMMARY OF THE INVENTION

The present disclosure can be taken to generally relate to the determination of a subject's immune competence. In one aspect, the invention relates to the identification of one or more subjects that/who are at lower or higher risk for developing PML. More specifically, the present invention provides inter alia a method for assessing the likelihood that a subject will develop a condition associated with JC virus and a method of stratification for risk of a JCV induced disease. Typically such a method is a method of PML risk stratification. A respective subject may be in an immunosuppressive condition. A respective subject may also have received a bone marrow transplant, an organ transplant, or a stem cell transplant. In one aspect, this disclosure provides a method of risk assessment of an individual such as a patient that undergoes α₄-integrin blocking agent treatment and/or VLA-4 blocking agent treatment to occurrence of a JCV-induced disease or at least some aspects of such disease. In a further aspect the invention provides a method of detecting or diagnosing risk of PML occurrence as well as a method for diagnosis and/or prognosis of PML. In another aspect this disclosure provides a method for determining whether an individual such as a patient infected with a retrovirus such as HIV is or is not at an increased risk of suffering from a JCV-induced disease. In yet a further aspect there is provided a method of performing flow cytometry on T cells from an individual in order to assess the likelihood that the individual will or will not develop a JCV-induced disease. The present disclosure provides biomarkers the level of which can assist a practitioner in determining an appropriate therapeutic regimen for a subject, typically a patient. The present invention also provides a method of treating a subject infected with HIV as well as a method of treating a subject suffering from an autoimmune disorder. In some embodiments the autoimmune disorder is a pathological inflammatory disease, such as MS, Crohn's disease, sarcoidosis, Sjögren's syndrome, Churg-Strauss syndrome or ulcerative colitis. In some embodiments the autoimmune disorder is Graves' disease, idiopathic thrombocytopenic purpura, Addison's disease, Hashimoto's thyroiditis, systemic lupus erythematosus or an idiopathic inflammatory myopathy such as dermatomyositis, polymyositis and sporadic inclusion body myositis.

The methods and uses provided by the present invention are based on employing L-selectin (CD62L), P-selectin glycoprotein ligand-1 (PSGL-1) and/or lymphocyte function-associated antigen-1 (LFA-1) as a biomarker for identifying a predisposition of a subject of developing PML. In the context of the present invention CD62L levels, PSGL-1 levels and/or LFA-1 levels may be determined using any desired technique. In some embodiments means may be employed that indirectly indicate CD62L levels, PSGL-1 levels and/or LFA-1 levels, for example by assessing indicators from which levels of CD62L, PSGL-1 and/or LFA-1 can be inferred. A method according to the invention may include assigning a likelihood of one or more future changes in a subject's immune competence, in particular with regard to a subject's risk of having a condition associated with JC virus. A method according to the invention may include staging, monitoring, categorizing and/or determination of a subject's immune competence, as well as staging, monitoring, categorizing and/or determination of further diagnosis and treatment regimens in a subject at risk of suffering from a JCV-induced disease.

According to a first aspect, the present invention provides a method of assessing the risk of occurrence of PML in a subject. The method generally includes providing a sample from the subject. Further the method includes detecting the level of PSGL-1 expressing T cells in the sample from the subject. In some embodiments the method further includes detecting the level of CD62L expressing T cells in the sample from the subject. In some embodiments the method further includes detecting the level of LFA-1 expressing T cells in the sample from the subject. In one embodiment the method includes detecting the level of CD62L expressing T cells, of LFA-1 expressing T cells and of PSGL-1 expressing T cells in the sample from the subject.

According to some embodiments of the method according to the first aspect the sample is a body fluid sample from the subject selected from a blood sample, a lymph sample and a sample of cerebrospinal fluid.

According to a particular embodiment of the method according to the first aspect, the T cells are CD3⁺ T cells. According to particular embodiments of the method according to the first aspect, the subject is suffering from a retroviral infection. The subject may for example be infected with HIV. In one such embodiment the method includes detecting the level of CD62L expressing T cells in a sample from the subject. According to a particular embodiment of the method according to the first aspect, the expression is monitored at certain, e.g. predetermined, time intervals.

According to a further embodiment of the method according to the first aspect, the subject has been diagnosed as being in need of treatment with an α₄-integrin and/or a VLA-4 blocking agent. In such an embodiment the level of CD62L expressing T cells, PSGL-1 expressing T cells and/or LFA-1 expressing T cells in the sample from the subject may be analysed.

According to yet a further embodiment of the method according to the first aspect, the subject is undergoing treatment with α₄-integrin blocking agent treatment and/or a VLA-4 blocking agent. The α₄-integrin/VLA-4 blocking agent is in some embodiments an immunoglobulin or a proteinaceous binding molecule with immunoglobulin-like functions. According to an embodiment of the method according to the first aspect, a decreased level of CD62L expressing T cells, of LFA-1 expressing T cells and/or of PSGL-1 expressing T cells, relative to a threshold value, may indicate an elevated risk of occurrence of PML. In such embodiments a method according to the first aspect may include determining that the subject is at an elevated risk of occurrence of PML. According to this embodiment of the method according to the first aspect, an increased level of CD62L expressing T cells, of LFA-1 expressing T cells and/or of PSGL-1 expressing T cells, relative to a threshold value, or a level of CD62L expressing T cells, of LFA-1 expressing T cells and/or of PSGL-1 expressing T cells that is at about the threshold value may indicate no elevated risk of occurrence of PML. In this case it may accordingly be determined that the subject is not at an elevated risk of occurrence of PML.

In some embodiments of the method according to the first aspect it is further determined whether the subject is seropositive for JCV, that is whether immunoglobulins against JCV are present in the subject's organism. If the subject is not seropositive for JCV it is determined that the subject is not at elevated risk of occurrence of PML. If the subject is seropositive for JCV, that is the subject has immunoglobulins against JCV, and a decreased level of PSGL-1 expressing T cells, relative to a threshold value, is detected, it is determined that the subject is at an elevated risk of developing a condition associated with JCV infection. In embodiments where the level of CD62L expressing T cells and/or of LFA-1 expressing T cells in the sample is detected, and the subject is seropositive for JCV, if a decreased level of one of CD62L expressing T cells, LFA-1 expressing T cells, and PSGL-1 expressing T cells, relative to a threshold value, is detected, it is determined that the subject is at an elevated risk of developing a condition associated with JCV infection.

In some embodiments of the method according to the first aspect detecting the level of CD62L expressing T cells, of LFA-1 expressing T cells and/or of PSGL-1 expressing T cells includes contacting the sample with a binding partner. The binding partner is specific for at least one of CD62L, LFA-1 and PSGL-1, respectively. In such embodiments of the method according to the first aspect detecting the level of CD62L expressing T cells, of LFA-1 expressing T cells and/or of PSGL-1 expressing T cells further includes detecting the amount of the binding partner that is binding to proSP-B.

In some embodiments the method according to the first aspect includes carrying out flow cytometry on T cells from the subject. In some embodiments the method according to the first aspect includes carrying out flow cytometry to sort T cells. Sorting T cells may be based on contacting cells in a sample from the subject with a binding partner. The binding partner may in some embodiments be immobilized on a substrate. In some embodiments the binding partner is in solution. In some embodiments the binding partner is coupled to a detectable label. In one embodiment the method includes carrying out fluorescence assisted cell sorting (FACS).

According to some embodiments of the method according to the first aspect, the method includes comparing the level of CD62L, LFA-1 and/or PSGL-1 expressing T cells in the sample to a threshold value.

In some embodiments of the method according to the first aspect a decreased level of CD62L expressing T cells, of LFA-1 expressing T cells and/or of PSGL-1 expressing T cells, relative to a threshold value, indicates an elevated risk of occurrence of PML. A level of CD62L expressing T cells, of LFA-1 expressing T cells and/or of PSGL-1 expressing T cells that is about at a threshold value or above a threshold value indicates no elevated risk of occurrence of PML when compared to healthy subjects. In some embodiments a method according to the first aspect accordingly includes diagnosing the likelihood of occurrence or nonoccurrence of PML, and the level of CD62L expressing T cells, of LFA-1 expressing T cells and/or of PSGL-1 expressing T cells is/are correlated to the likelihood of occurrence or nonoccurrence of PML.

In some embodiments of the method according to the first aspect an increased risk of occurrence of PML is determined if a decreased level of CD62L expressing T cells, of LFA-1 expressing T cells and/or of PSGL-1 expressing T cells, relative to a threshold value, is detected. In some embodiments of the method according to the first aspect it is determined that no increased risk of occurrence of PML exists if a level of CD62L expressing T cells, of LFA-1 expressing T cells and/or of PSGL-1 expressing T cells is detected that is about at a threshold value or above a threshold value.

In one embodiment more than one level of the level of CD62L expressing T cells, of LFA-1 expressing T cells and of PSGL-1 expressing T cells is determined. Each of the measured levels/amounts may be compared to a threshold value. An increased likelihood of the occurrence of PML is assigned to the subject when the measured concentration is below the threshold (relative to the likelihood assigned when the measured concentration is above or at the threshold). When the measured concentration is above or at the threshold, an increased likelihood of the nonoccurrence of PML may be assigned to the subject (relative to the likelihood assigned when the measured concentration is below the threshold).

In some embodiments the method according to the first aspect is included in a method of treating a subject with an autoimmune disease, including a demyelinating disease. As explained above, PML is thought to be caused by JCV, so that the corresponding demyelinating disease treated is different from PML. Examples of a respective autoimmune disease include, but are not limited to, multiple sclerosis, including relapsing-remitting MS and secondary progressive MS, Crohn's disease, rheumatoid arthritis and psoriasis. The method of treating a subject with an autoimmune disease includes administering a VLA-4 blocking agent, determining the expression of PSGL-1 on T cells of the subject, and continuing or discontinuing the administration of the α₄-integrin-blocking agent based on the determined level of PSGL-1 expression. Determining the expression of PSGL-1 on T cells of the subject is typically carried out on T cells that are included in a sample from the subject treated or to be treated. The administration of the VLA-4 blocking agent may be stopped if a decreased level of PSGL-1 expressing T cells relative to a threshold value is identified. The administration of the α₄-integrin-blocking agent may be continued if a level of PSGL-1 expressing T cells is determined that is about the same as a threshold value, or above a threshold value. In some embodiments such a method further includes determining the expression of CD62L and/or LFA-1 on T cells of the subject. In such an embodiment the administration of the α₄-integrin-blocking agent may be stopped or continued based on the measured level of expression of PSGL-1 as well as CD62L and/or LFA-1 on T cells of the subject. The administration of the α₄-integrin-blocking agent may be discontinued if a decreased level of at least one of PSGL-1 expressing T cells, CD62L expressing T cells and LFA-1 expressing T cells, relative to a threshold value is determined. The administration of the α₄-integrin-blocking agent may be continued if a level of PSGL-1, CD62L and/or LFA-1 expressing T cells is determined that is about the same as a threshold value, or above a threshold value.

In some embodiments the method according to the first aspect is included in a method of treating patients with a retroviral infection, including a HIV infection. The method of treating a subject with a retroviral infection includes administering an antiretroviral compound or a combination of antiretroviral compounds, determining the expression of PSGL-1 on T cells of the subject, and continuing or discontinuing the administration of the antiretroviral compound(s) based on the determined level of PSGL-1 expression. Determining the expression of PSGL-1 on T cells of the subject is typically carried out on T cells that are included in a sample from the subject treated or to be treated. The administration of the antiretroviral compound(s) may be stopped if a decreased level of PSGL-1 expressing T cells relative to a threshold value is identified. The administration of the antiretroviral compound(s) may be continued if a level of PSGL-1 expressing T cells is determined that is about the same as a threshold value, or above a threshold value. In some embodiments such a method further includes determining the expression of CD62L and/or LFA-1 on T cells of the subject. In such an embodiment the administration of the antiretroviral compound(s) may be stopped or continued based on the measured level of expression of PSGL-1 as well as CD62L and/or LFA-1 on T cells of the subject. The administration of antiretroviral compound(s) may be discontinued if a decreased level of at least one of PSGL-1 expressing T cells, CD62L expressing T cells and LFA-1 expressing T cells, relative to a threshold value is determined. The administration of the antiretroviral compound(s) may be continued if a level of PSGL-1, CD62L and/or LFA-1 expressing T cells is determined that is about the same as a threshold value, or above a threshold value.

According to further embodiments of the method according to the first aspect the method includes determining the migration of CD45⁺CD49d⁺ immune cells, such as CD45⁺CD49d⁺ T cells. In some embodiments migration is measured using a transendothelial chemotaxis assay. In some embodiments migration is measured using a chemotaxis assay, for instance employing a blank permeable membrane.

In a related second aspect the invention provides a method of screening one or more individuals for risk or future occurrence of a condition associated with JCV infection. In some embodiments one or more of the one or more individuals is/arc infected with a retrovirus such as HIV. The method generally includes providing a sample from each of the one or more subjects. The method includes detecting the level of PSGL-1 expressing T cells in the sample from each of the one or more subjects. In some embodiments the method further includes detecting the level of CD62L expressing T cells in the sample from each of the one or more subjects. In some embodiments the method further includes detecting the level of LFA-1 expressing T cells in the sample from each of the one or more subjects. In one embodiment the method includes detecting the level of CD62L expressing T cells, detecting the level of LFA-1 expressing T cells and detecting the level of PSGL-1 expressing T cells in the sample from each of the one or more subjects.

According to an embodiment of the method according to the second aspect, the method includes comparing the level of CD62L, LFA-1 and/or PSGL-1 expressing T cells in the sample to a threshold value.

In some embodiments of the method according to the second aspect an altered, such as a decreased or an increased, level of CD62L, LFA-1 and/or PSGL-1 expressing T cells, relative to a threshold value, may indicate an increased risk of future occurrence of a condition associated with JCV infection. In such embodiments a method according to the second aspect may include determining that the subject is at an increased risk of future occurrence of a condition associated with JCV infection.

In one embodiment of the method according to the second aspect a decreased level of CD62L expressing T cells, of LFA-1 expressing T cells and/or of PSGL-1 expressing T cells, relative to a threshold value, indicates that the subject is at an increased risk of future occurrence of a condition associated with JCV infection. A level of CD62L expressing T cells, of LFA-1 expressing T cells and/or of PSGL-1 expressing T cells that is about at a threshold value or above a threshold value indicates that the subject is not at an increased risk of future occurrence of a condition associated with JCV infection when compared to healthy subjects.

In some embodiments of the method according to the second aspect it is determined that a subject is at an increased risk of future occurrence of a condition associated with JCV infection if a decreased level of PSGL-1 expressing T cells, relative to a threshold value, is detected. In some embodiments of the method according to the second aspect it is determined that a subject is at increased risk of a condition associated with JCV infection if a decreased level of at least one of PSGL-1 expressing T cells and CD62L expressing T cells, relative to a threshold value, is detected.

In some embodiments of the method according to the second aspect it is further determined whether the subject is seropositive for JCV, i.e. whether the subject carries immunoglobulins against JCV. If the subject is not seropositive for JCV it is determined that the subject is not at an elevated risk of developing a condition associated with JCV infection. If the subject is seropositive for JCV, that is the subject has immunoglobulins against JCV, and a decreased level of PSGL-1 expressing T cells, relative to a threshold value, is detected, it is determined that the subject is at an elevated risk of developing a condition associated with JCV infection.

In some embodiments of the method according to the second aspect, the T cells are CD3⁺ T cells. In some embodiments the method according to the second aspect includes carrying out flow cytometry to sort T cells. Sorting T cells may be based on contacting cells in a sample from the subject with a binding partner. The binding partner may in some embodiments be immobilized on a substrate. In some embodiments the binding partner is in solution. In some embodiments the binding partner is coupled to a detectable label. In one embodiment the method includes carrying out FACS.

According to some embodiments of the method according to the second aspect the method includes determining the migration of CD45⁺CD49d⁺ immune cells, such as CD45⁺CD49d⁺ T cells. In some embodiments migration is measured using a transendothelial chemotaxis assay. In some embodiments migration is measured using a chemotaxis assay, for instance employing a blank permeable membrane.

In a third aspect there is provided a method of monitoring the risk of occurrence of a JCV related condition in a subject. The method includes monitoring the level of PSGL-1 expressing T cells of the subject. In some embodiments such method further includes monitoring the level of CD62L and/or LFA-1 expressing T cells of the subject. Generally these T cells are included, including provided, in a sample from the subject. In some embodiments of the method according to the third aspect, the T cells are CD3⁺ T cells. Monitoring the expression of CD62L, PSGL-1 and/or LFA-1 on T cells is generally carried out using a sample from the subject. Monitoring may be carried out at predetermined time intervals. In some embodiments monitoring begins prior to a treatment. A respective treatment may be a treatment for improving the immune competence of the subject, such as HAART (supra). In some embodiments a respective treatment may be an α₄-integrin-blocking agent treatment such as a VLA-4 blocking agent treatment and/or a LPAM-1 blocking agent treatment.

In some embodiments the method according to the third aspect includes carrying out flow cytometry to sort T cells. Sorting T cells may be based on contacting cells in a sample from the subject with a binding partner. The binding partner may in some embodiments be immobilized on a substrate. In some embodiments the binding partner is in solution. In some embodiments the binding partner is coupled to a detectable label. In one embodiment the method includes carrying out FACS.

According to further embodiments of the method according to the third aspect, the method includes determining the migration of CD45⁺CD49d⁺ immune cells, such as CD45⁺CD49d⁺ T cells. In some embodiments migration is measured using a transendothelial chemotaxis assay. In some embodiments migration is measured using a chemotaxis assay, for instance employing a blank permeable membrane.

In a related fourth aspect there is provided a method of monitoring the risk of occurrence of PML in a subject. The method includes monitoring the level of expression of PSGL-1 on T cells. In some embodiments the method of the fourth aspect further includes monitoring the level of expression of CD62L and/or LFA-1 on T cells. Generally these T cells are included, including provided, in a sample from the subject.

In one embodiment of the method according to the fourth aspect a decreased level of CD62L expressing T cells, of LFA-1 expressing T cells and/or of PSGL-1 expressing T cells at a point of time, relative to a threshold value, indicates that the subject is at an elevated risk to suffer from JCV, including a condition associated with JCV infection. In some embodiments a decreased level of CD62L expressing T cells, of LEA-1 expressing T cells and/or of PSGL-1 expressing T cells at two consecutive points of time (when a measurement was performed), relative to a threshold value, indicates that the subject is at an elevated risk to suffer from JCV. A level of CD62L expressing T cells, of LFA-1 expressing T cells and/or of PSGL-1 expressing T cells that is about at a threshold value or above a threshold value indicates that the subject is not at an elevated risk to suffer from a condition associated with JCV infection when compared to healthy subjects.

In some embodiments of the method according to the fourth aspect, the T cells are CD3 T cells. In some embodiments the method according to the fourth aspect includes carrying out flow cytometry to sort T cells. Sorting T cells may be based on contacting cells in a sample from the subject with a binding partner. The binding partner may in some embodiments be immobilized on a substrate. In some embodiments the binding partner is in solution. In some embodiments the binding partner is coupled to a detectable label. In one embodiment the method includes carrying out FACS.

In some embodiments the method according to the fourth aspect includes determining the migration of CD45⁺CD49d⁺ immune cells, such as CD45⁺CD49d⁺ T cells. In some embodiments migration is measured using a transendothelial chemotaxis assay. In some embodiments migration is measured using a chemotaxis assay, for instance employing a blank permeable membrane.

In a fifth aspect there is disclosed a method of screening patients who are known or suspected to be prone to occurrence of PML, i.e. susceptible to PML. The method generally includes detecting the level of PSGL-1 expressing T cells in a sample from the subject. In some embodiments the method further includes detecting the level of CD62L and/or LFA-1 expressing T cells in a sample from the subject. The method may also include comparing the result, the level of PSGL-1 expressing T cells, as well as—where applicable—CD62L and/or LFA-1 expressing T cells, to a threshold value.

According to a sixth aspect in this regard, the invention provides a method of monitoring the risk of occurrence of a JCV related complication of AIDS/HIV infection. The method includes monitoring the level of CD62L expressing T cells and/or PSGL-1 expressing T cells in a sample from a subject having AIDS/HIV infection. Generally these T cells are included, including provided, in one or more samples from the subject. In some embodiments the T cells are CD3⁺ T cells.

According to a particular embodiment of the method according to the sixth aspect, the expression is monitored at certain, e.g. predetermined, time intervals. Samples from the subject may be provided that have been obtained at the corresponding time points.

According to an embodiment of the method according to the sixth aspect, the method includes comparing the level of CD62L, LFA-1 and/or PSGL-1 expressing T cells in the sample to a threshold value.

In some embodiments the method according to the sixth aspect includes carrying out flow cytometry to sort T cells. Sorting T cells may be based on contacting cells in a sample from the subject with a binding partner. The binding partner may in some embodiments be immobilized on a substrate. In some embodiments the binding partner is in solution. In some embodiments the binding partner is coupled to a detectable label. In one embodiment the method includes carrying out FACS.

In some embodiments of the method according to the sixth aspect an altered, such as a decreased or an increased, level of CD62L and/or PSGL-1 expressing T cells, relative to a threshold value, may indicate an increased risk of occurrence of a condition associated with JCV infection. According to a particular embodiment, the method according to the sixth aspect includes comparing the level of CD62L and/or PSGL-1 expressing T cells in the sample to a threshold value.

In one embodiment of the method according to the sixth aspect a decreased level of CD62L expressing T cells, of LFA-1 expressing T cells and/or of PSGL-1 expressing T cells, relative to a threshold value, indicates that the subject is at or has acquired an increased risk to suffer from a condition associated with JCV infection. A level of CD62L expressing T cells, of LFA-1 expressing T cells and/or of PSGL-1 expressing T cells that is about at a threshold value or above a threshold value indicates that the subject is not at an increased risk to suffer from a condition associated with JCV infection when compared to healthy subjects.

In some embodiments the method according to the sixth aspect includes determining the migration of CD45⁺CD49d⁺ immune cells, such as CD45⁺CD49d⁺ T cells. In some embodiments migration is measured using a transendothelial chemotaxis assay. In some embodiments migration is measured using a chemotaxis assay, for instance employing a blank permeable membrane.

According to a seventh aspect, the invention provides a method of predicting the risk of occurrence of PML in a subject. The method can also be taken to be a method of predicting whether a patient is at risk of developing PML. The method includes detecting the level of T cells expressing PSGL-1 in a sample from the subject. The method generally includes providing a sample from the subject. The method further includes detecting the level of T cells expressing PSGL-1 in the sample. In some embodiments the T cells are CD3⁺ T cells.

In some embodiments the method according to the seventh aspect includes comparing the expression of PSGL-1 on T cells to a reference value or to a threshold level. A threshold level may be based on one or more reference values.

In some embodiments the method according to the seventh aspect further includes detecting the level of T cells expressing CD62L in a sample from the subject. In some embodiments the method according to the seventh aspect includes comparing the expression of CD62L on T cells to a reference value or to a threshold level. A threshold level may be based on one or more reference values. According to a particular embodiment of the method according to the seventh aspect, the expression of CD62L and PSGL-1 is monitored at certain, e.g. predetermined, time intervals.

In some embodiments of the method according to the seventh aspect a decreased level of at least one of CD62L expressing T cells and PSGL-1 expressing T cells, relative to a threshold value, may indicate an elevated risk of occurrence of PML in the subject. In such embodiments a method according to the seventh aspect may include determining that the subject is at an elevated risk of occurrence of PML. According to a particular embodiment of the method according to the seventh aspect, the level of CD62L expressing T cells and of PSGL-1 expressing T cells in the sample is compared to a threshold value.

In one embodiment of the method according to the seventh aspect a decreased level of at least one of CD62L expressing T cells and of PSGL-1 expressing T cells, relative to a threshold value, indicates an elevated risk of occurrence of PML in the subject. A level of at least one of CD62L expressing T cells and of PSGL-1 expressing T cells that is about at a threshold value or above a threshold value indicates no elevated risk of occurrence of PML in the subject when compared to healthy subjects.

According to a particular embodiment of the method according to the seventh aspect the method further includes detecting the level of T cells expressing LFA-1 in the sample. According to a particular embodiment of the method according to the seventh aspect the subject is infected with a retrovirus. The subject may for example be HIV positive. According to a particular embodiment of the method according to the seventh aspect the subject is undergoing treatment with an [symbol] ₄-integrin-blocking agent such as a VLA-4 blocking agent and/or a LPAM-1 blocking agent. The [symbol] ₄-integrin-blocking agent is in some embodiments an immunoglobulin or a proteinaceous binding molecule with immunoglobulin-like functions.

In some embodiments the method according to the seventh aspect includes carrying out flow cytometry to sort T cells. Sorting T cells may be based on contacting cells in a sample from the subject with a binding partner. The binding partner may in some embodiments be immobilized on a substrate. In some embodiments the binding partner is in solution. In some embodiments the binding partner is coupled to a detectable label. In one embodiment the method includes carrying out FACS.

In some embodiments the method according to the seventh aspect includes determining the migration of CD45⁺CD49d⁺ immune cells, such as CD45⁺CD49d⁺ T cells. In some embodiments migration is measured using a transendothelial chemotaxis assay. In some embodiments migration is measured using a chemotaxis assay, for instance employing a blank permeable membrane.

According to an eighth aspect, the invention provides a method of monitoring the risk of occurrence of a JCV related complication under treatment with an α₄-integrin-blocking agent. The α₄-integrin-blocking agent is in some embodiments an immunoglobulin or a proteinaceous binding molecule with immunoglobulin-like functions. The method includes monitoring the level of T cells in a sample from a subject having AIDS/HIV infection, which express CD62L, LFA-1 and/or PSGL-1. Generally these T cells are included, including provided, in one or more samples from the subject. Samples from the subject may have been obtained at certain time points.

In some embodiments of the method according to the eighth aspect, the T cells are CD3⁺ T cells. In some embodiments the method according to the eighth aspect includes carrying out flow cytometry to sort T cells (cf. also above). In one embodiment the method includes carrying out FACS.

In some embodiments the method according to the eighth aspect includes comparing the level of CD62L, LFA-1 and/or PSGL-1 expressing T cells in the sample to a threshold value. In one embodiment of the method according to the eighth aspect a decreased level of CD62L expressing T cells, of LFA-1 expressing T cells and/or of PSGL-1 expressing T cells, relative to a threshold value, at a point of time indicates that the subject is at an elevated risk of occurrence of a JCV related complication. In some embodiments a decreased level of CD62L expressing T cells, of LFA-1 expressing T cells and/or of PSGL-1 expressing T cells at two consecutive points of time (where a measurement was performed), relative to a threshold value, indicates that the subject is at an elevated risk of occurrence of a JCV related complication. A level of CD62L expressing T cells, of LFA-1 expressing T cells and/or of PSGL-1 expressing T cells that is about at a threshold value or above a threshold value indicates that the subject is not at an elevated risk of occurrence of a JCV related complication when compared to healthy subjects.

According to a particular embodiment, the method according to the eighth aspect includes monitoring the migration of CD45⁺CD49d⁺ immune cells, such as CD45⁺CD49d⁺ T cells. In some embodiments migration is measured using a transendothelial chemotaxis assay. In some embodiments migration is measured using a chemotaxis assay employing a blank permeable membrane.

According to a ninth aspect, the invention provides a method of stratifying a subject that/who is undergoing α₄-integrin blocking agent treatment for suspension of α₄-integrin blocking agent treatment. The α₄-integrin-blocking agent is in some embodiments an immunoglobulin or a proteinaceous binding molecule with immunoglobulin-like functions. The method generally includes providing a sample from the subject. The method further includes detecting the level of T cells in the sample from the subject, with the T cells expressing PSGL-1. In some embodiments the T cells, the level of which is detected, are expressing CD62L and/or LFA-1. In some embodiments of the method according to the ninth aspect, the T cells are CD3⁺ T cells.

In some embodiments of the method according to the ninth aspect the sample is a body fluid sample from the subject selected from a blood sample, a lymph sample and a sample of cerebrospinal fluid.

In some embodiments the method according to the ninth aspect is a method of screening subjects under treatment with an α₄-integrin blocking agent as to whether they are more prone to PML.

In some embodiments the method according to the ninth aspect includes carrying out flow cytometry to sort T cells. Sorting T cells may be based on contacting cells in a sample from the subject with a binding partner. The binding partner may in some embodiments be immobilized on a substrate. In some embodiments the binding partner is in solution. In some embodiments the binding partner is coupled to a detectable label. In one embodiment the method includes carrying out FACS. In one embodiment the method includes carrying out FACS.

According to a particular embodiment, the method according to the ninth aspect includes comparing the level of CD62L, LFA-1 and/or PSGL-1 expressing T cells in the sample to a threshold value.

In some embodiments of the method according to the ninth aspect a decreased level of CD62L, LFA-1 and/or PSGL-1 expressing T cells, relative to a threshold value, may indicate an increased risk of occurrence of PML. In such embodiments a method according to the ninth aspect may include stratifying the subject for suspension of α₄-integrin blocking agent treatment.

In one embodiment of the method according to the ninth aspect a decreased level of CD62L expressing T cells, of LFA-1 expressing T cells and/or of PSGL-1 expressing T cells, relative to a threshold value, indicates an increased risk of occurrence of PML. A level of CD62L expressing T cells, of LFA-1 expressing T cells and/or of PSGL-1 expressing T cells that is about at a threshold value or above a threshold value indicates no increased risk of occurrence of PML when compared to healthy subjects.

According to a tenth aspect, the invention provides a method of stratifying a subject undergoing HAART for suspension of HAART. The method generally includes providing a sample from the subject. The method further includes detecting the level of CD62L expressing T cells and/or of PSGL-1 expressing T cells in the sample from the subject.

In some embodiments of the method according to the tenth aspect the sample is a body fluid sample from the subject selected from a blood sample, a lymph sample and a sample of cerebrospinal fluid.

In some embodiments of the method according to the tenth aspect an altered, such as a decreased or an increased, level of CD62L and/or of PSGL-1 expressing T cells, relative to a threshold value, may indicate an elevated risk of occurrence of PML. In such embodiments a method according to the tenth aspect may include stratifying the subject for suspension of HAART. According to a particular embodiment, the method according to the tenth aspect includes comparing the level of CD62L expressing T cells and/or of PSGL-1 expressing T cells in the sample to a threshold value.

In one embodiment of the method according to the tenth aspect a decreased level of CD62L expressing T cells or of PSGL-1 expressing T cells, relative to a threshold value, indicates an elevated risk of occurrence of PML in the subject. A level of at least one of CD62L expressing T cells and of PSGL-1 expressing T cells that is about at a threshold value or above a threshold value indicates no elevated risk of occurrence of PML in the subject when compared to healthy subjects. If a decreased level of CD62L expressing T cells or of PSGL-1 expressing T cells, relative to a threshold value, is detected, a subject undergoing HAART may be stratified for suspension of HAART. If a level of CD62L expressing T cells or of PSGL-1 expressing T cells is detected that is about at a threshold value or above a threshold value the subject may not be stratified for suspension of HAART.

In some embodiments of the method according to the tenth aspect the T cells are CD3 T cells. In some embodiments the method according to the tenth aspect includes carrying out flow cytometry to sort T cells. Sorting T cells may be based on contacting cells in a sample from the subject with a binding partner. The binding partner may in some embodiments be immobilized on a substrate. In some embodiments the binding partner is in solution. In some embodiments the binding partner is coupled to a detectable label. In one embodiment the method includes carrying out FACS.

In some embodiments the method according to the tenth aspect is a method of screening subjects under HAART as to whether they are more prone to develop PML.

According to some embodiments, the method according to the tenth aspect includes determining the migration of CD45⁺CD49d⁺ immune cells, such as CD45⁺CD49d⁺ T cells. In some embodiments migration is measured using a transendothelial chemotaxis assay. In some embodiments migration is measured using a chemotaxis assay, for instance employing a blank permeable membrane.

According to an eleventh aspect, the invention provides a method of determining the proportion, such as the percentage, of T cells of a subject that have PSGL-1 on the cell surface. Typically the method is carried out on a sample from the subject. The method includes determining the ratio of T cells that have PSGL-1 on the cell surface to the total number of T cells, for example T cells in a sample from the subject. The method includes contacting the T cells with a binding partner specific for PSGL-1. The method further includes allowing the formation of a complex between PSGL-1 on the T cells and the binding partner.

In some embodiments of the method according to the eleventh aspect the T cells are CD3⁺ T cells. In some embodiments the method according to the eleventh aspect includes comparing the proportion of PSGL-1 expressing T cells to a threshold value. In one embodiment of the method according to the eleventh aspect, if a decreased proportion of PSGL-1 expressing T cells, relative to a threshold value, is detected, it is determined that the subject is (a) in need of a therapy to prevent the occurrence of a condition associated with JCV infection or (b) in need of a change of HIV therapy or α₄-integrin-blocking agent therapy so as to avoid the occurrence of a condition associated with JCV infection. The α₄-integrin blocking agent is in some embodiments an immunoglobulin or a proteinaceous binding molecule with immunoglobulin-like functions. As applicable, the subject is then exposed to a therapy to prevent the occurrence of a condition associated with JCV infection. If under such therapy, a HIV therapy or an α₄-integrin blocking agent therapy is changed. If an increased proportion of PSGL-1 expressing T cells, relative to a threshold value, or a proportion of about the threshold value is detected, it is determined that the subject is (a) not in need of a therapy to prevent the occurrence of a condition associated with JCV infection or (b) not in need of a change of HIV therapy or α₄-integrin blocking agent therapy.

In some embodiments of the method according to the eleventh aspect it is further determined whether the subject is seropositive for JCV. If the subject is not seropositive for JCV any HIV therapy or an α₄-integrin blocking agent therapy is continued. No therapy to prevent the occurrence of a condition associated with JCV infection is initiated. If the subject is seropositive for JCV and a decreased proportion of PSGL-1 expressing T cells, relative to a threshold value, is detected, e.g. in a sample from the subject, a HIV therapy or a α₄-integrin blocking agent therapy is changed, if applicable. If the subject is seropositive for JCV and a decreased proportion of PSGL-1 expressing T cells, relative to a threshold value, is detected, the subject may also be exposed to a therapy to prevent the occurrence of a condition associated with JCV infection.

Typically the T cells from the subject are included in a sample from the subject. In some embodiments of the method according to the eleventh aspect the sample is a body fluid sample from the subject selected from a blood sample, a lymph sample and a sample of cerebrospinal fluid.

According to a twelfth aspect, the invention provides a method of determining the proportion, such as the percentage, of T cells of a subject that have CD62L, LFA-1 and/or PSGL-1 on the cell surface. The subject has AIDS/HIV infection. Typically the method is carried out on a sample from the subject. The method includes determining the ratio of T cells that have CD62L, LFA-1 and/or PSGL-1 on the cell surface to the total number of T cells, for example T cells in the sample. The method includes contacting the T cells with a binding partner specific for at least one of CD62L, LFA-1 and PSGL-1, respectively. The method further includes allowing the formation of a complex between CD62L, LFA-1 and/or PSGL-1 on the T cells and the binding partner.

In some embodiments the method according to the twelfth aspect includes comparing the proportion of CD62L, LFA-1 and/or PSGL-1 expressing T cells to a threshold value. In one embodiment of the method according to the twelfth aspect, if a decreased proportion of CD62L expressing T cells, of LFA-1 expressing T cells and/or of PSGL-1 expressing T cells, relative to a threshold value, is detected, it is determined that the subject is in need of a change of HIV therapy so as to avoid the occurrence of a condition associated with JCV infection. Any HIV therapy is changed accordingly. If an increased proportion of CD62L, LFA-1 and/or PSGL-1 expressing T cells, relative to a threshold value, or a proportion of about the threshold value is detected, it is determined that the subject is not in need of a change of HIV therapy.

In some embodiments of the method according to the twelfth aspect it is further determined whether the subject is seropositive for JCV. If the subject is not seropositive for JCV any HIV therapy is continued. If the subject is seropositive for JCV and a decreased proportion of CD62L, LFA-1 and/or PSGL-1 expressing T cells, relative to a threshold value, is detected, e.g. in a sample from the subject, a HIV therapy is changed.

In some embodiments of the method according to the twelfth aspect the T cells are CD3⁺ T cells. Typically the T cells from the subject are included in a sample from the subject. In some embodiments of the method according to the twelfth aspect the sample is a body fluid sample from the subject selected from a blood sample, a lymph sample and a sample of cerebrospinal fluid.

According to a thirteenth aspect, the invention relates to a method of carrying out flow cytometry on T cells from a subject. The method is generally a diagnostic method. The method includes contacting the T cells with a binding partner specific for PSGL-1. The method further includes allowing the formation of a complex between PSGL-1 on the T cells and the binding partner. In typical embodiments the method of the thirteenth aspect includes allowing the T cells to pass through a microfluidic device that is capable of interrogating the T cells with regard to the presence of PSGL-1. In some embodiments the microfluidic device has a sensor that is capable of detecting the binding partner. The method further includes determining the number of PSGL-1 expressing T cells relative to the total number of T cells in the sample. The method further includes comparing the number of PSGL-1 expressing T cells, relative to the total number of T cells, to a threshold value.

In some embodiments of the method according to the thirteenth aspect the T cells are CD3⁺ T cells. Typically the T cells from the subject are included in a sample from the subject. In some embodiments of the method according to the thirteenth aspect the sample is a body fluid sample from the subject selected from a blood sample, a lymph sample and a sample of cerebrospinal fluid.

According to a fourteenth aspect, the invention relates to a method of carrying out flow cytometry on T cells from a subject having AIDS/HIV infection. The method is generally a diagnostic method. The method includes contacting the T cells with one or more binding partners specific for at least one of CD62L, LFA-1 and PSGL-1. The method further includes allowing the formation of a complex between CD62L, LFA-1 and/or PSGL-1 on the T cells and the corresponding binding partner. In typical embodiments the method of the fourteenth aspect includes allowing the T cells to pass through a microfluidic device that is capable of interrogating the T cells with regard to the presence of CD62L, LFA-1 and/or PSGL-1. In some embodiments the microfluidic device has a sensor that is capable of detecting the binding partner. The method further includes determining the number of CD62L, LFA-1 and/or PSGL-1 expressing T cells relative to the total number of T cells in the sample. The method further includes comparing the number of CD62L, LFA-1 and/or PSGL-1 expressing T cells, relative to the total number of T cells, to a threshold value.

In some embodiments of the method according to the fourteenth aspect the T cells are CD3⁺ T cells. Typically the T cells from the subject are included in a sample from the subject. In some embodiments of the method according to the fourteenth aspect the sample is a body fluid sample from the subject selected from a blood sample, a lymph sample and a sample of cerebrospinal fluid.

According to a fifteenth aspect, the invention relates to the in-vitro use of a binding partner, which is specific for CD62L, for assessing the risk of occurrence of PML in a subject. The subject may in some embodiments suffer from a retroviral infection. In some embodiments the subject is infected with HIV.

In typical embodiments the binding partner is an immunoglobulin or a proteinaceous binding molecule with immunoglobulin-like functions, with the binding molecule or the immunoglobulin being specific for CD62L.

According to a particular embodiment of the use according to the fifteenth aspect the subject may undergo treatment with one or more α₄-integrin-blocking agents. In some embodiments the use according to the fifteenth aspect includes carrying out flow cytometry to sort CD62L+ T cells. Sorting T cells may be based on contacting cells in a sample from the subject with a binding partner. The binding partner may in some embodiments be immobilized on a substrate. In some embodiments the binding partner is in solution. In some embodiments the binding partner is coupled to a detectable label. In one embodiment the method includes carrying out FACS. In one embodiment the method includes carrying out FACS.

According to a sixteenth aspect, the invention relates to the in-vitro use of a binding partner, which is specific for CD62L, for stratifying a subject undergoing HAART for alteration of HAART.

In typical embodiments the binding partner is an immunoglobulin or a proteinaceous binding molecule with immunoglobulin-like functions specific for CD62L. In some embodiments the use according to the sixteenth aspect includes carrying out flow cytometry to sort CD62L+ T cells. In one embodiment the method includes carrying out FACS.

According to a seventeenth aspect, the invention relates to the in-vitro use of a binding partner, which is specific for PSGL-1, for assessing the risk of occurrence of PML in a subject. The subject is infected with a retrovirus such as HIV.

In typical embodiments the binding partner is an immunoglobulin or a proteinaceous binding molecule with immunoglobulin-like functions specific for PSGL-1.

According to an eighteenth aspect, the invention relates to the in-vitro use of a binding partner, which is specific for PSGL-1, for stratifying a subject undergoing HAART for alteration of HAART.

In typical embodiments the binding partner is an immunoglobulin or a proteinaceous binding molecule with immunoglobulin-like functions specific for PSGL-1. In some embodiments the use according to the eighteenth aspect includes carrying out flow cytometry to sort PSGL-1+ T cells. Sorting T cells may be based on contacting cells in a sample from the subject with a binding partner. The binding partner may in some embodiments be immobilized on a substrate. In some embodiments the binding partner is in solution. In some embodiments the binding partner is coupled to a detectable label. In one embodiment the method includes carrying out FACS.

According to a nineteenth aspect the invention relates to the in-vitro use of a binding partner, which is specific for LFA-1, for assessing the risk of occurrence of PML in a subject. The subject is infected with a retrovirus such as HIV.

In typical embodiments the binding partner is an immunoglobulin or a proteinaceous binding molecule with immunoglobulin-like functions specific for LFA-1. In some embodiments the use according to the nineteenth aspect includes carrying out flow cytometry to sort LFA-1+ T cells. In one embodiment the method includes carrying out FACS.

According to a twentieth aspect, the invention relates to the in-vitro use of a binding partner, which is specific for at least one of CD11A, CD18 and LFA-1, for assessing the risk of occurrence of PML in a subject. The subject is suffering from a retroviral infection. In some embodiments the subject is infected with HIV.

In typical embodiments the binding partner is an immunoglobulin or a proteinaceous binding molecule with immunoglobulin-like functions specific for at least one of CD11A, CD18 and LFA-1. In some embodiments the method according to the twentieth aspect includes carrying out flow cytometry to sort T cells positive for at least one of CD11A, CD18 and LFA-1. Sorting T cells may be based on contacting cells in a sample from the subject with a binding partner, which is specific for CD11A, CD18 and/or LFA-1, respectively. The binding partner may in some embodiments be immobilized on a substrate. In some embodiments the binding partner is in solution. In some embodiments the binding partner is coupled to a detectable label. In one embodiment the method includes carrying out FACS.

According to a twenty-first aspect the invention relates to the use of a PSGL-1 binding assay kit for determining the risk of a subject undergoing VLA-4 blocking agent treatment to develop PML.

According to a particular embodiment of the use according to the twenty-first aspect, the PSGL-1 binding assay kit employs a PSGL-1 binding partner.

According to a twenty-second aspect the invention relates to the use of a CD62L and/or PSGL-1 binding assay kit for determining the risk of a subject infected with a retrovirus, for instance an HIV positive subject, to develop PML.

According to a particular embodiment of the use according to the twenty-second aspect, the CD62L binding assay kit employs a CD62L binding partner. According to a particular embodiment of the use according to the twenty-second aspect, the PSGL-1 binding assay employs a PSGL-1 binding partner.

According to a twenty-third aspect, the invention relates to the measurement of one or more biomarkers selected from the group consisting of CD62L, LFA-1 and PSGL-1 for the prognosis of PML in a subject. The twenty-third aspect may also be taken to relate to the use of one or more biomarkers selected from the group consisting of CD62L, LEA-1 and PSGL-1 for the prognosis of PML in a subject. In some embodiments the use/measurement is for the evaluation of the risk of occurrence of PML in the subject.

Typically the measurement of the one or more biomarkers is carried out on a sample from the subject. In some embodiments the measurement of the one or more biomarkers is determining whether the one or more biomarkers are present on the surface of T cells. In some embodiments of the method according to the twenty-third aspect the T cells are CD3⁺ T cells. Typically these T cells are included in a sample from the subject. In some embodiments of the measurement or use according to the twenty-third aspect the sample is a body fluid sample from the subject selected from a blood sample, a lymph sample and a sample of cerebrospinal fluid.

In some embodiments the measurement or use according to the twenty-third aspect includes carrying out flow cytometry to sort T cells. In one embodiment the measurement/use includes carrying out FACS.

According to a twenty-fourth aspect, the invention provides a method of stratifying a subject infected with a retrovirus such as HIV for discontinuing the administration of one or more anti-retroviral compounds. The subject is accordingly under treatment with these anti-retroviral compounds. The method generally includes providing a sample from the subject. The method further includes detecting the level of T cells expressing CD62L, T cells expressing LFA-1 and/or T cells expressing PSGL-1 in a sample from the subject. In some embodiments of the method according to the twenty-fourth aspect an altered, such as a decreased level of at least one of CD62L expressing T cells, LFA-1 expressing T cells and PSGL-1 expressing T cells, relative to a threshold value, may indicate the need to discontinue the administration of the anti-retroviral compounds. In such embodiments a method according to the twenty-fourth aspect may include determining that the subject is at an elevated risk of occurrence of PML. According to a particular embodiment of the method according to the twenty-fourth aspect, the level of CD62L expressing T cells and of PSGL-1 expressing T cells in the sample is compared to a threshold value.

According to a particular embodiment of the method according to the twenty-fourth aspect, the expression of CD62L, LFA-1 and PSGL-1 is monitored at certain, e.g. predetermined, time intervals. In some embodiments of the method according to the twenty-fourth aspect the T cells are CD3⁺ T cells. In some embodiments the method according to the aspect includes carrying out flow cytometry to sort T cells. In one embodiment the measurement/use includes carrying out FACS.

In some embodiments the method according to the twenty-fourth aspect is a method of screening subjects infected with a retrovirus and under treatment with one or more anti-retroviral compounds for an alteration of antiretroviral therapy.

According to a twenty-fifth aspect, the invention provides a method of treating a retroviral infection in a subject so as to avoid the additional occurrence of PML. The method includes administering a combination of anti-retroviral compounds to the subject, generally an effective amount of the anti-retroviral compounds, over a period of time, followed by discontinuing the administration for a period of time. Discontinuing administration of the combination of anti-retroviral compounds is effected if a reduced level of T cells that express PSGL-1 is determined. Accordingly the method generally includes measuring the amount/the proportion of T cells that express PSGL-1. In some embodiments discontinuing administration of the combination of anti-retroviral compounds includes starting administration of an alternative combination of anti-retroviral compounds. In some embodiments the method includes administering a combination of anti-retroviral compounds to the subject over a period of time, followed by exchanging the combination of anti-retroviral compounds administered for a different combination of anti-retroviral compounds.

In some embodiments of the method according to the twenty-fifth aspect it is further determined whether the subject is seropositive for JCV. If the subject is not seropositive for JCV administering the combination of anti-retroviral compounds is continued. If the subject is seropositive for JCV and a decreased level of PSGL-1 expressing T cells, relative to a threshold value, is detected in a sample from the subject, administering the combination of anti-retroviral compounds is discontinued for a period of time.

In a related twenty-sixth aspect the invention provides a combination of anti-retroviral compounds for use in the treatment of retroviral infection so as to avoid the additional occurrence of PML. The use includes administration of the combination to a subject over a period of time, followed by a discontinuation of the administration for a period of time.

According to a twenty-seventh aspect, the invention provides a method of treating a retroviral infection in a subject. The method includes administering a combination of anti-retroviral compounds to the subject, generally an effective amount of the anti-retroviral compounds. The method further includes measuring the level of expression of one or more of CD62L, LFA-1 and PSGL-1 on T cells, such as CD3⁺ T cells, of the subject, typically in a sample from the subject. In some embodiments the method includes repeatedly determining the expression of the level of CD62L, LFA-1 and/or PSGL-1 in a sample from the subject. In some embodiments the method includes monitoring CD62L expressing T cells, LFA-1 expressing T cells and/or PSGL-1 expressing T cells in a sample from the subject. Based on the amount of T cells expressing CD62L, LFA-1 and/or PSGL-1 that has been determined, the administration of the combination of anti-retroviral compounds is stopped or continued. Stopping administration of the combination of anti-retroviral compounds may include starting administration of an alternative combination of anti-retroviral compounds. In some embodiments the administration of the combination of anti-retroviral compounds is replaced by administration of a different combination of anti-retroviral compounds. The method according to the twenty-seventh aspect may include discontinuing the administration of the anti-retroviral compounds for a period of time if a decreased level of CD62L expressing T cells and/or PSGL-1 expressing T cells relative to a threshold value is determined. The method according to the twenty-seventh aspect may include continuing the administration of the anti-retroviral compounds if a level of CD62L expressing T cells and/or PSGL-1 expressing T cells is determined that is a level being at about a threshold value or a level above a threshold value.

In some embodiments of the method according to the twenty-seventh aspect the amount of T cells that express CD62L, LFA-1 and/or PSGL-1 is determined after administration of the anti-retroviral compounds has been discontinued. In some embodiments the amount of T cells that express CD62L, LFA-1 and/or PSGL-1 is monitored after administration of the anti-retroviral compounds has been discontinued. If it is determined that the number of T cells that express CD62L, LFA-1 and/or PSGL-1 has recovered, i.e. increased relative to one or more previous values after discontinuing administration of the anti-retroviral compound(s), the anti-retroviral compound(s) may be administered to the subject. In some embodiments administration of the anti-retroviral compound(s) is started again if a level of T cells expressing CD62L, LFA-1 and/or PSGL-1 is determined that is about at the level of a threshold value or above a threshold value.

According to a particular embodiment, the method according to the twenty-seventh aspect further includes comparing the level of CD62L and/or PSGL-1 expressing T cells in the sample to a threshold value. According to another particular embodiment, the method according to the twenty-seventh aspect further includes determining migration of immune cells, such as CD45⁺CD49⁺ cells and T cells. In some embodiments the method may include both (i) detecting the level of expression of the one or more biomarkers on T cells and (ii) determining migration of immune cells.

According to a further particular embodiment, the method according to the twenty-seventh aspect further includes discontinuing administering the combination of anti-retroviral compounds if an altered, such as a decreased or an increased, level of CD62L and/or PSGL-1 expressing T cells has been determined. In one embodiment discontinuing administering the combination includes a substitution therapy. Discontinuing administering the combination may include administering a further combination of anti-retroviral compounds. This further combination is different from the combination of anti-retroviral compounds used initially. Thus in one embodiment the method includes administering a first combination of anti-retroviral compounds to the subject, generally an effective amount of the anti-retroviral compounds of the first combination. The method further includes monitoring the expression of the level of CD62L and/or PSGL-1 expressing T cells in a sample from the subject. The method may further include discontinuing administering the first combination of anti-retroviral compounds and beginning administering a second combination of anti-retroviral compounds if an altered level of CD62L and/or PSGL-1 expressing T cells has been determined. The second combination of anti-retroviral compounds is different from the first combination of anti-retroviral compounds.

In some embodiments of the method according to the twenty-seventh aspect it is further determined whether the subject is seropositive for JCV. If the subject is not seropositive for the administration of the anti-retroviral compounds administration of the anti-retroviral compounds is continued. If the subject is seropositive for JCV and a decreased level of CD62L expressing T cells and/or PSGL-1 expressing T cells, relative to a threshold value, is detected, the administration of the anti-retroviral compounds is discontinued for a period of time.

According to a particular embodiment of the method according to the twenty-seventh aspect the retroviral infection is a HIV infection.

According to a twenty-eighth aspect the invention provides a method of treating a subject that/who is in a state of immunodeficiency so as to avoid the additional occurrence of PML. In some embodiments the method of the twenty-eighth aspect is a method of treating a subject that/who is suffering from an autoimmune disease, including a demyelinating disease. The subject may for instance be suffering from MS, e.g. relapsing-remitting MS and secondary progressive MS, Crohn's disease and/or rheumatoid arthritis. The method includes administering one or more α₄-integrin blocking agents, such as VLA-4 blocking agents, to the subject, generally an effective amount of the α₄-integrin blocking agent(s), over a period of time. The α₄-integrin blocking agent is in some embodiments an immunoglobulin or a proteinaceous binding molecule with immunoglobulin-like functions. The method further includes measuring the level of expression of T cells, such as CD3⁺ T cells and/or CD4⁺ T cells, that express PSGL-1 of the subject, typically in a sample from the subject. In some embodiments the method includes repeatedly determining the expression of the level of PSGL-1 on T cells in a sample from the subject. In some embodiments the method includes monitoring PSGL-1 expressing T cells in a sample from the subject. Based on the level of T cells expressing PSGL-1 that has been determined, the administration of the α₄-integrin-blocking agent(s) is stopped or continued. Accordingly the method may include discontinuing the administration of the α₄-integrin-blocking agent(s) for a period of time. Discontinuing the administration of the one or more α₄-integrin-blocking agents may be effected after a decreased level of PSGL-1 expressing T cells relative to a threshold value is determined. The method according to the twenty-eighth aspect may include discontinuing the administration of the α₄-integrin blocking agent(s) for a period of time if a decreased level of PSGL-1 expressing T cells relative to a threshold value is determined. The method according to the twenty-eighth aspect may include continuing the administration of the [symbol] ₄-integrin blocking agent(s) if a level of PSGL-1 expressing T cells is determined that is a level, which is at about a threshold value or a level that is above a threshold value.

The method may further include monitoring PSGL-1 expression levels on T cells, including CD3⁺ T cells and/or CD4⁺ T cells, in a sample from the subject, after administration of the α₄-integrin blocking agent(s) has been discontinued. If it is determined that the number of T cells that express PSGL-1 has recovered, i.e. increased relative to one or more previous values after discontinuing administration of the α₄-integrin blocking agent(s), the α₄-integrin blocking agent(s) may be administered to the subject. In some embodiments administration of the α₄-integrin blocking agent(s) is started again if a level of T cells expressing PSGL-1 is determined that is about at the level of a threshold value or above a threshold value.

In some embodiments the subject is suffering from an autoimmune disease such as a demyelinating disease. The subject may for instance be suffering from MS, e.g. relapsing-remitting MS and secondary progressive MS, Crohn's disease, rheumatoid arthritis and/or psoriasis.

In some embodiments of the method according to the twenty-eighth aspect it is further determined whether the subject is seropositive for JCV. If the subject is not seropositive for JCV, administering the α₄-integrin-blocking agent is continued. If the subject is seropositive for JCV and a decreased level of PSGL-1 expressing T cells, relative to a threshold value, is detected in a sample from the subject, administering the α₄-integrin-blocking agent is discontinued for a period of time.

In some embodiments of the method according to the twenty-eighth aspect is a method of treating an autoimmune disease in a subject. In some embodiments the subject is suffering from a retroviral infection such as HIV.

According to a twenty-ninth aspect the invention provides a combination of an immunoglobulin or a proteinaceous binding molecule with immunoglobulin-like functions specific for CD62L, an immunoglobulin or a proteinaceous binding molecule with immunoglobulin-like functions specific for CD3⁺, and an immunoglobulin or a proteinaceous binding molecule with immunoglobulin-like functions specific for HIV.

According to a particular embodiment, the combination according to the twenty-ninth aspect is provided in the form of a kit. The kit includes a first container that includes the immunoglobulin or a proteinaceous binding molecule with immunoglobulin-like functions specific for CD62L. The kit further includes a second container that includes the immunoglobulin or a proteinaceous binding molecule with immunoglobulin-like functions specific for CD3⁺. The kit also includes a third container that includes the immunoglobulin or a proteinaceous binding molecule with immunoglobulin-like functions specific for HIV.

According to a thirtieth aspect the invention provides a combination of an immunoglobulin or a proteinaceous binding molecule with immunoglobulin-like functions specific for PSGL-1, an immunoglobulin or a proteinaceous binding molecule with immunoglobulin-like functions specific for CD62L, and an immunoglobulin or a proteinaceous binding molecule with immunoglobulin-like functions specific for CD3⁺.

According to a particular embodiment, the combination according to the thirtieth aspect is provided in the form of a kit. The kit includes a first container that includes the immunoglobulin or a proteinaceous binding molecule with immunoglobulin-like functions specific for PSGL-1. The kit further includes a second container that includes the immunoglobulin or a proteinaceous binding molecule with immunoglobulin-like functions specific for CD62L. The kit also includes a third container that includes the immunoglobulin or a proteinaceous binding molecule with immunoglobulin-like functions specific for CD3⁺.

According to a thirty-first aspect there is provided a method of treating a subject. The method includes administering one or more α₄-integrin-blocking agents and/or VLA-4 blocking agents to the subject. In some embodiments the α₄-integrin-blocking agent is an immunoglobulin or a proteinaceous binding molecule with immunoglobulin-like functions. The method may further include detecting the level of expression of PSGL-1 on T cells, such as CD3⁺ T cells, of the subject. In some embodiments the method further includes detecting the level of CD62L expressing T cells in the sample from the subject. In some embodiments the method further includes detecting the level of LFA-1 expressing T cells in the sample from the subject. In one embodiment the method includes detecting the level of CD62L expressing T cells, of LFA-1 expressing T cells and of PSGL-1 expressing T cells in the sample from the subject. In some embodiments the expression of PSGL-1 and, where applicable, of CD62L and/or LFA-1 on T cells is monitored. Generally these T cells are included, including provided, in a sample from the subject. The method may also include determining migration of immune cells, such as CD45⁺CD49⁺ cells and T cells. In some embodiments, a respective biomarker is one or more of CD62L, PSGL-1 and LFA-1. In some embodiments the method may include both (i) detecting the level of expression of the one or more biomarkers on T cells and (ii) determining migration of immune cells. In some embodiments the subject may be suffering from an autoimmune disorder. In some embodiments the autoimmune disorder may be a demyelinating disorder.

The subject is in some embodiments suffering from a pathologic inflammatory disease within the CNS. The subject may in some embodiments be diagnosed to have an autoimmune disease, such as multiple sclerosis, e.g. relapsing-remitting multiple sclerosis and secondary progressive multiple sclerosis or Crohn's disease. In some embodiments the VLA-blocking agent is CD29d specific, i.e. specific for the integrin β1 chain. In some embodiments the VLA-blocking agent is CD49d specific, i.e. specific for the integrin α4 chain. Examples of a suitable VLA-4 blocking agent include, but are not limited to, the monoclonal antibodies Natalizumab, HP2/1, HP1/3, HP1/2, including humanized HP1/2, HP1/7, HP2/4, B-5G10, TS2/16, L25, P4C2, AJM300 and the recombinant anti-VLA4 immunoglobulins described in U.S. Pat. No. 6,602,503 and U.S. Pat. No. 7,829,092, a low molecular weight VLA-4 antagonist such as SB-683699, a CS-1 peptidomimetic as disclosed in e.g. U.S. Pat. No. 5,821,231, U.S. Pat. No. 5,869,448, U.S. Pat. No. 5,869,448, U.S. Pat. No. 5,936,065, U.S. Pat. No. 6,265,572, U.S. Pat. No. 6,288,267, U.S. Pat. No. 6,365,619, U.S. Pat. No. 6,423,728, U.S. Pat. No. 6,426,348, U.S. Pat. No. 6,458,844, U.S. Pat. No. 6,479,666, U.S. Pat. No. 6,482,849, U.S. Pat. No. 6,596,752, U.S. Pat. No. 6,667,331, U.S. Pat. No. 6,668,527, U.S. Pat. No. 6,685,617, U.S. Pat. No. 6,903,128 or U.S. Pat. No. 7,015,216, a phenylalanine derivative as disclosed in e.g. U.S. Pat. No. 6,197,794, U.S. Pat. No. 6,229,011, U.S. Pat. No. 6,329,372, U.S. Pat. No. 6,388,084, U.S. Pat. No. 6,348,463, U.S. Pat. No. 6,362,204, U.S. Pat. No. 6,380,387, U.S. Pat. No. 6,445,550, U.S. Pat. No. 6,806,365, U.S. Pat. No. 6,835,738, U.S. Pat. No. 6,855,706, U.S. Pat. No. 6,872,719, U.S. Pat. No. 6,878,718, U.S. Pat. No. 6,911,451, U.S. Pat. No. 6,916,933, U.S. Pat. No. 7,105,520, U.S. Pat. No. 7,153,963, U.S. Pat. No. 7,160,874, U.S. Pat. No. 7,193,108, U.S. Pat. No. 7,250,516 or U.S. Pat. No. 7,291,645, alphafeto protein, a beta-amino acid compound as disclosed in e.g. U.S. patent applications US 2004/0229859 or US 2006/0211630, a semi-peptide compound as disclosed in e.g. U.S. Pat. No. 6,376,538, the tripeptide Leu-Asp-Val and a pegylated molecule as disclosed in U.S. patent application US 2007/066533 or U.S. Pat. No. 6,235,711.

Determining the level of CD62L, LFA-1 and/or PSGL-1 expressing T cells in any of the above aspects and embodiments may include detecting the number, proportion, e.g. percentage and/or the absolute number of T cells in the sample from the subject that have CD62L, LFA-1 and/or PSGL-1 on the cell surface. Determining the level of CD62L, LFA-1 and/or PSGL-1 expressing T cells may also include detecting the amount or level of CD62L, LFA-1 and/or PSGL-1 present on T cells of the sample from the subject. Determining the level of CD62L expressing T cells may also include detecting, in T cells of the sample from the subject, the amount or level of nucleic acid formation from the SELL gene encoding CD62L. Determining the level of LFA-1 expressing T cells may also include detecting the amount or level of nucleic acid formation from the ITGAL gene encoding CD11A and/or the ITGB2 gene encoding CD18. Determining the level of CD62L, LFA-1 and/or PSGL-1 expressing T cells may also include detecting, in T cells of the sample from the subject, the amount or level of nucleic acid formation from the SELPLG gene encoding PSGL-1.

### BRIEF DESCRIPTION OF THE D WINGS

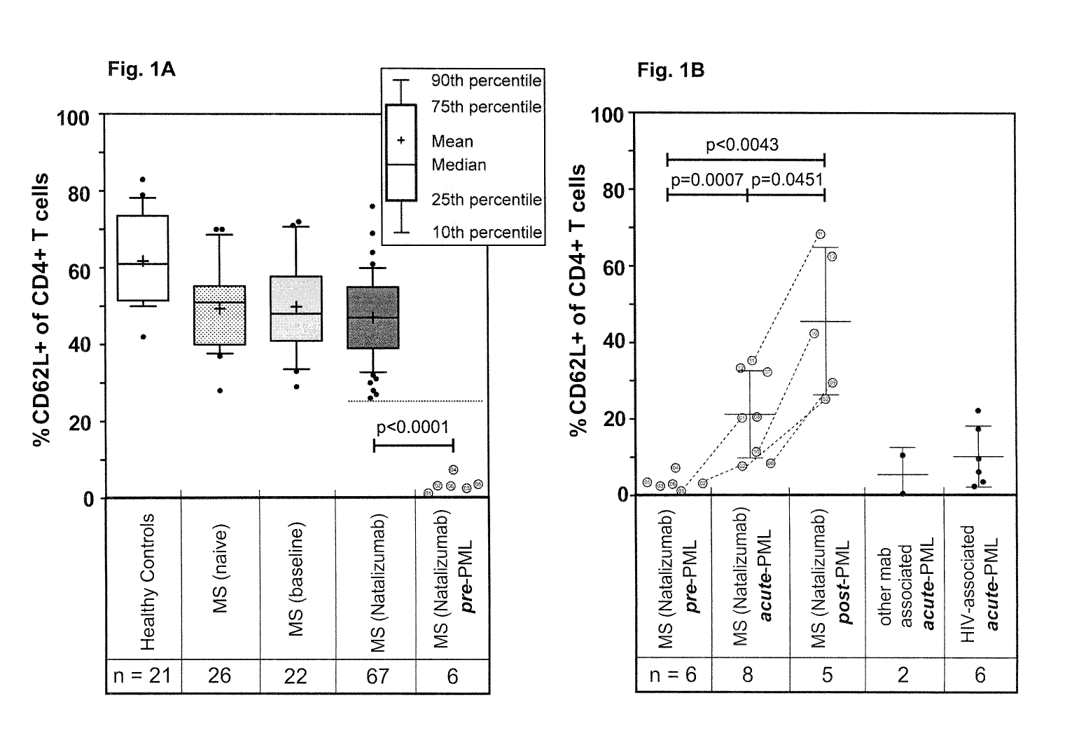

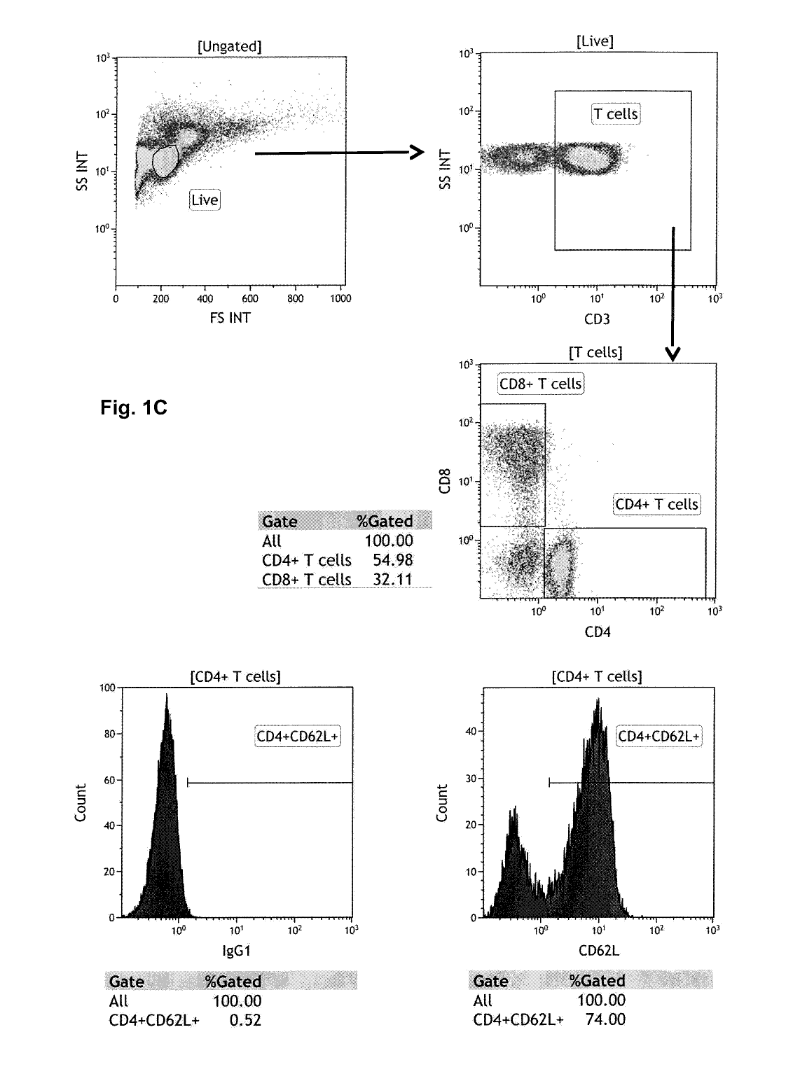

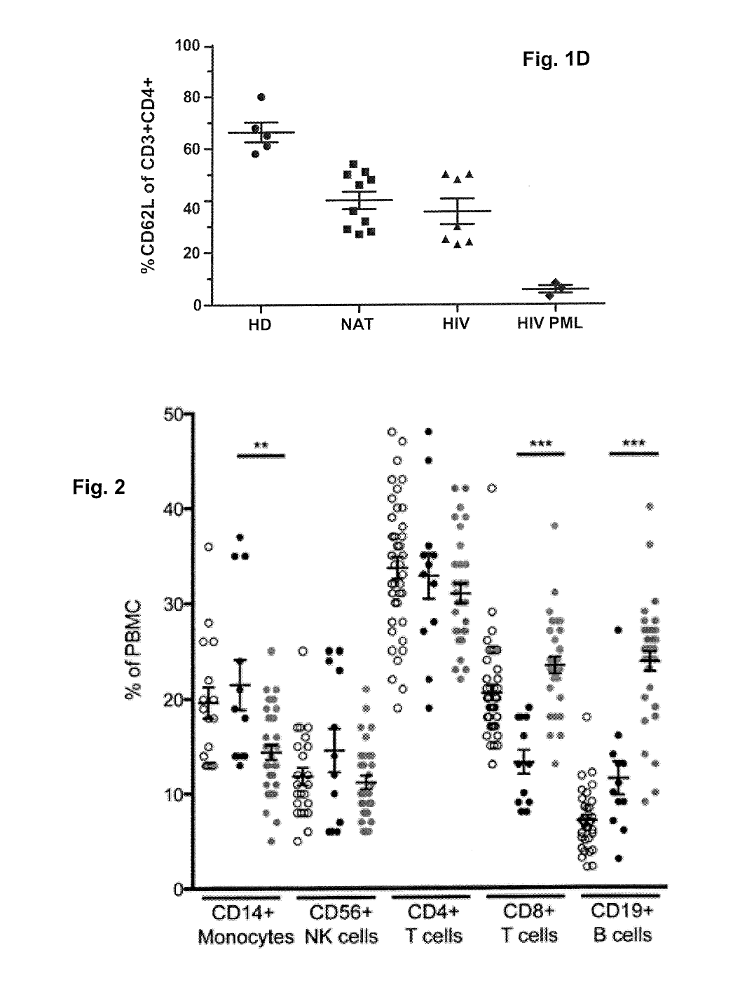

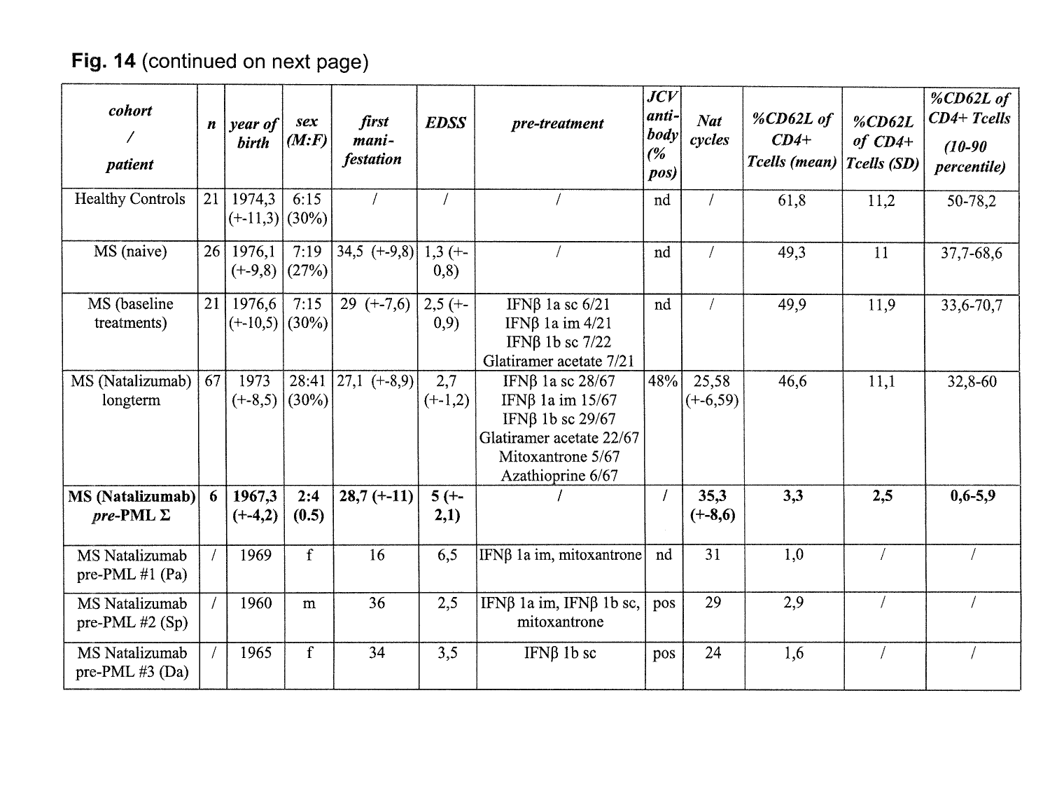

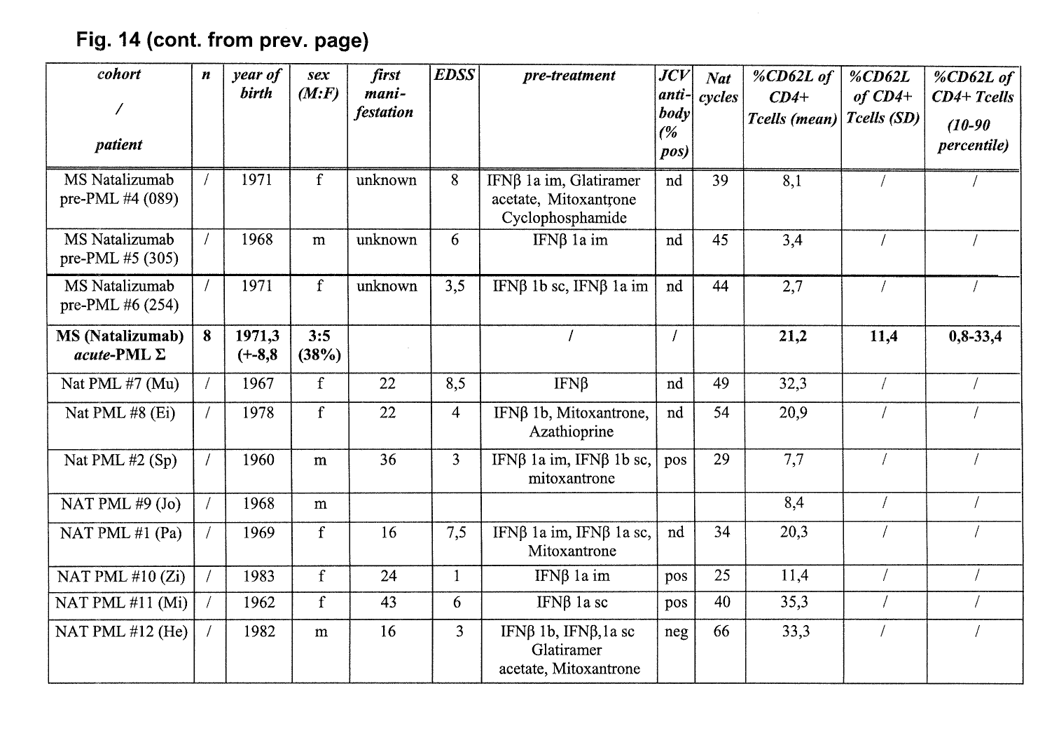

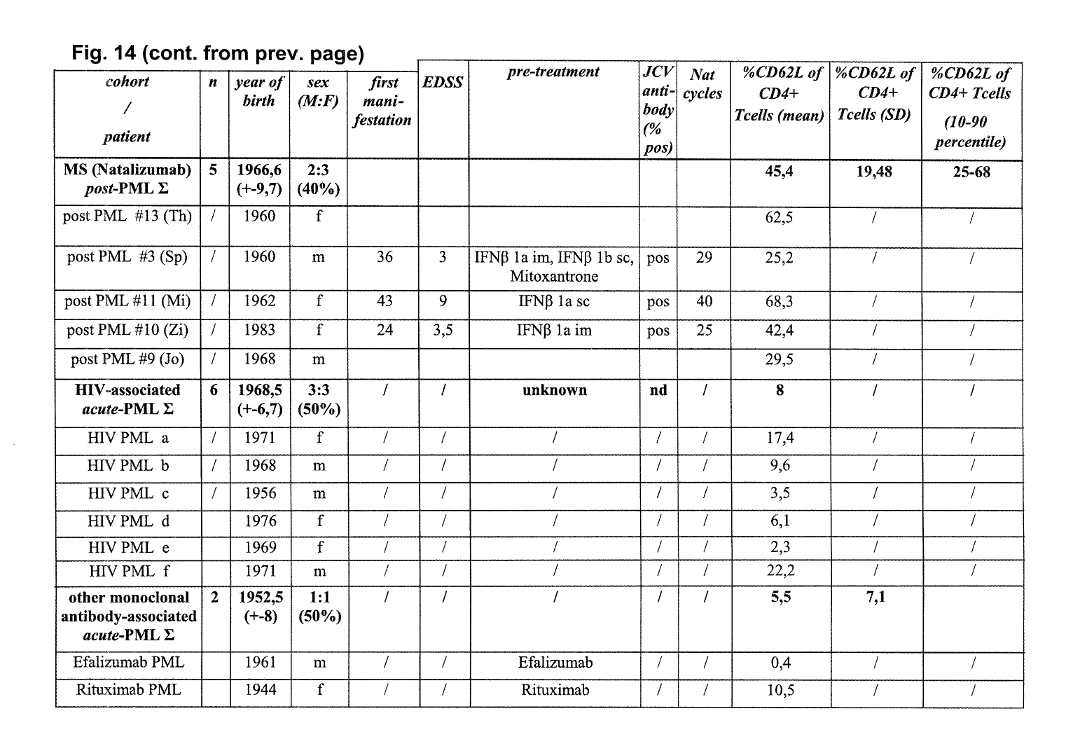

FIG. 1A depicts the percentage (%) of CD62L surface expressing CD3⁺ CD4⁺ T cells, as determined by flow cytometric measurements using peripheral blood derived mononuclear cells (PBMC). Cells were isolated from EDTA blood by density gradient centrifugation, frozen, thawed for analysis, and stained with fluorescence labeled immunoglobulins against CD3, CD4 and CD62L. Cells were gated as shown in FIG. 1C. The boxes in FIG. 1A represent 50% of each cohort (25^{th}-75^{th} percentile) while 80% of all individuals reside within the limits of each box and its whiskers (10^{th}-90^{th} percentile). The line within the boxes indicates the mean, the plus (+) represents the median of the respective cohort. Each dot represents an individual patient. The white box represents 21 control subjects without any acute or chronic disorder (healthy controls). The dotted box represents subjects diagnosed for MS, who are in stable condition and did not receive any prior immune-modulating treatment (MS naïve). The light grey box represents patients diagnosed for MS, who received baseline treatments other than Natalizumab as lined-out in FIG. 14. These blood withdrawings took place right before the escalation to Natalizumab therapy (MS baseline). The dark grey box indicates patients diagnosed for MS, who after receiving baseline treatments as lined-out in FIG. 14 received Natalizumab continuously for 18 months or longer (18-66 months of Natalizumab treatment, MS Natalizumab). The six numbered MS (Natalizumab) pre-PML patients all match the criteria of the dark grey cohort, but developed PML later on at different time points throughout Natalizumab long-term therapy as lined out in FIG. 14. The dotted line indicates the threshold for increased PML risk under long-term Natalizumab therapy (mean of the dark grey cohort minus two times its standard deviation).

FIG. 1B depicts the percentage (%) of CD62L surface expressing CD3+ CD4+ T cells, see the explanation for FIG. 1A for details. The MS (Natalizumab) acute- and post-PML cohorts both match the dark grey cohort but were sampled after PML onset, either while suffering from acute PML (MS (Natalizumab) acute-PML) or after PML subsided (MS (Natalizumab) post-PML, e.g. the beginning of immune reconstituation inflammatory syndrome (IRIS). Two patients with other monoclonal antibody-associated PMLs, one suffering from severe psoriasis treated with Efalizumab and one suffering from B-cell lymphoma treated with Rituximab (other monoclonal antibody-associated acute-PML), and seven HIV/AIDS PML patients (HIV-associated acute-PML) served as additional PML controls. The dashed lines indicate sequential samples, if identical patients were available at different time points during disease development.

FIG. 1C shows illustrative flow cytometry measurements with gating to life lymphocytes, CD3⁺ T cells as well as CD4⁺ and CD8⁺ T cells.

FIG. 1D depicts data of flow cytometric measurements of peripheral blood derived mononuclear cells (PBMC). Cells were isolated from EDTA blood (EDTA: 1.2 to 2 mg/ml blood) by density gradient centrifugation and subsequently frozen. For analysis, cells were thawed and stained with fluorescence labeled antibodies against CD3, CD4 and CD62L. 200,000 cells were used per staining. After flow cytometry measurement, cells were first gated to life lymphocytes, then CD3⁺ cells, then CD4⁺ cells and finally on CD62L⁺ cells (cf. also FIG. 1C). The graph depicts CD3⁺ CD4⁺ living lymphocytes that are positive for CD62L (1-selectin) expression. The groups are as follows: HD=healthy controls without any pathology or treatment; NAT=patients suffering from relapsing/remitting multiple sclerosis long-term treated with Natalizumab (18+ months of treatment); HIV=patients suffering from HIV infection treated with HAART medication; HIV PML=patients suffering from HIV infection treated with HAART medication that developed PML alongside therapy.

FIG. 2 shows percentages of CD14⁺ monocytes, CD4⁺ and CD8⁺ T cells, CD19+ B cells, and CD56⁺ NK cells (of PBMC) in peripheral blood of patients receiving long-term Natalizumab therapy (≧18 months). White dots represent healthy donors (n=16-39), black dots represent untreated MS patients (n=12), and grey dots represent Natalizumab patients treated ≧18 months continuously (n=34). Significance of differences between the groups is indicated by asterisks (*p<0.05, **p<0.01, ***p<0.001).

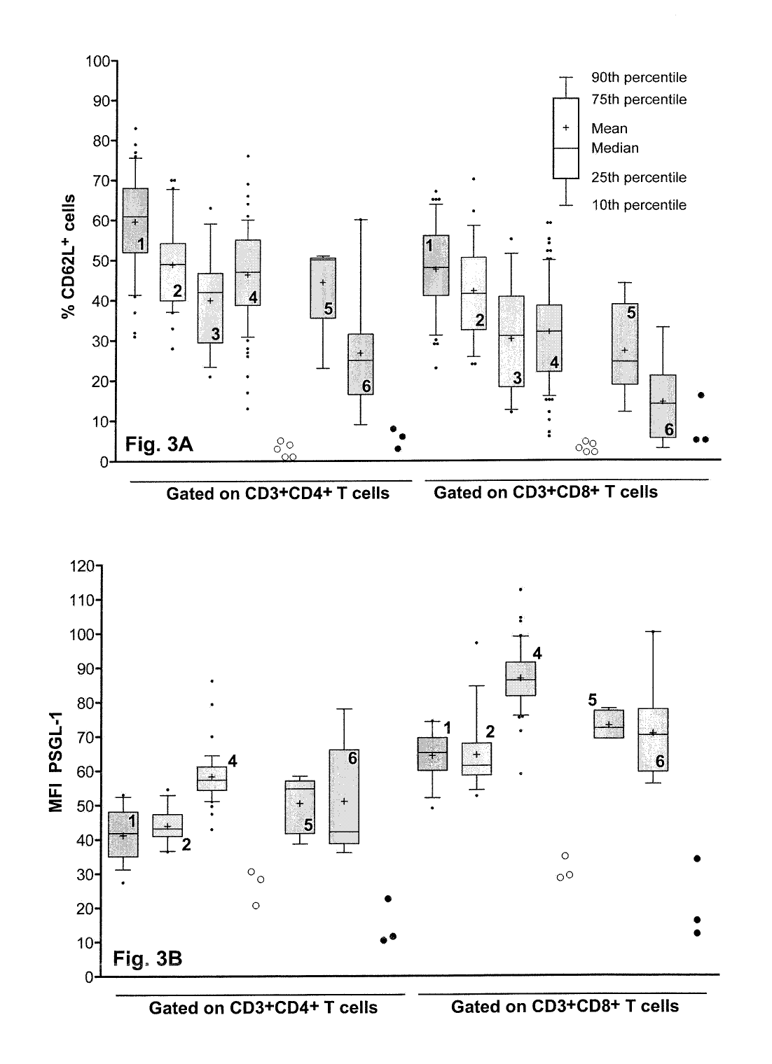

FIG. 3 depicts data analysis of flow cytometric measurements of peripheral blood derived mononuclear cells (PBMC). EDTA blood was obtained from patients and healthy control subjects as indicated above, PBMC were isolated by density gradient isolation and cryo-preserved in 50% RPMI, 40% FCS and 10% DMSO. Samples were subsequently thawed and stained in phosphate buffered saline (200 mM EDTA, 0.5% BSA) for surface markers (CD3, CD4, CD8, CD62L and CD162 (PSGL-1)). 1: Healthy controls, n=73; 2: Untreated RRMS patients, n=12; 3: RRMS patients before Natalizumab therapy, n=30; 4: RRMS patients after long-term Natalizumab therapy, which is defined as a therapy of more than 18 months, n=78; 5: HIV+ patients (CDC stadium B1-C2), n=5; 6: HIV+ patients (CDC stadium C3), n=9. White circles: RRMS patients under long-term Natalizumab therapy before onset of PML; Black circles: HIV+ patients after onset of PML.

FIG. 3A: The percentage of CD62L positive cells of CD3⁺CD4⁺ T cells or CD3⁺CD8⁺ T cells is shown. An isotype control was used to define a threshold between CD62L positive and negative cells.

FIG. 3B: PSGL-1 expression on CD3⁺CD4⁺ T cells or CD3⁺CD8⁺ T cells is shown; MFI=Mean Fluorescence Intensity.

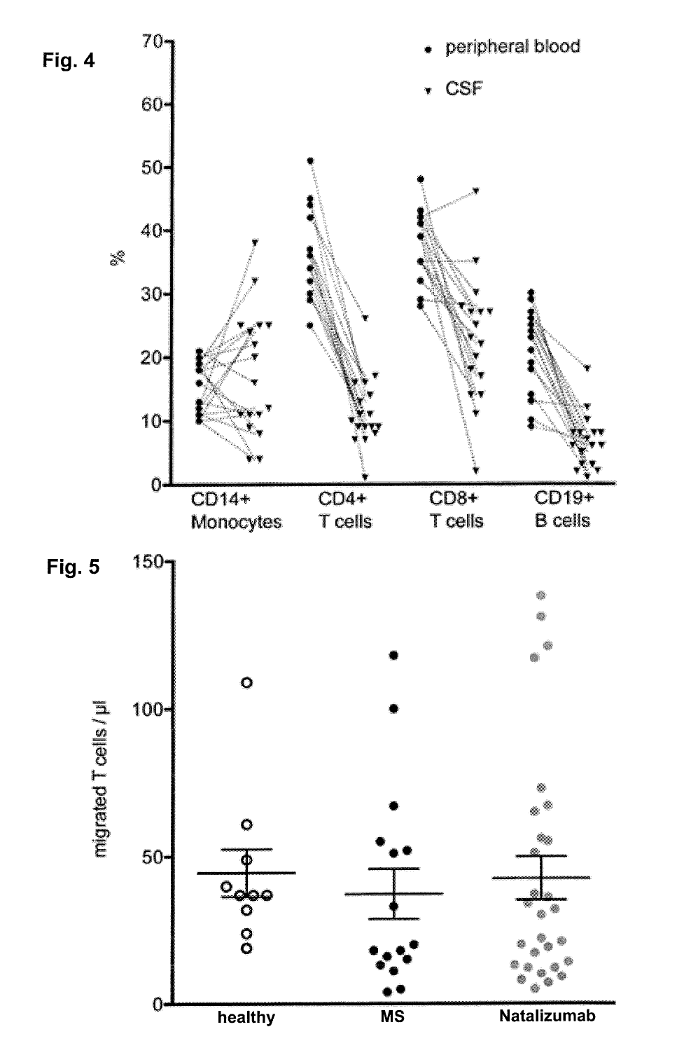

FIG. 4 shows the immune cell composition in peripheral blood (dots) and CSF (triangles) of patients under Natalizumab therapy (n=18; treatment ≧18 months). Given are percentages of monocytes, CD4⁺ and CD8⁺ T cells and B cells (of total leukocytes).

FIG. 5 shows the in vitro migration of isolated PBMC over primary human microvascular endothelial cells (HBMEC). Given are the absolute values of migrated T cells per μl of sample represented by individual dots of healthy donors (open circles, n=10), untreated MS patients (black, n=16) or Natalizumab patients (grey, n=29). Migration was assessed after 6 h.

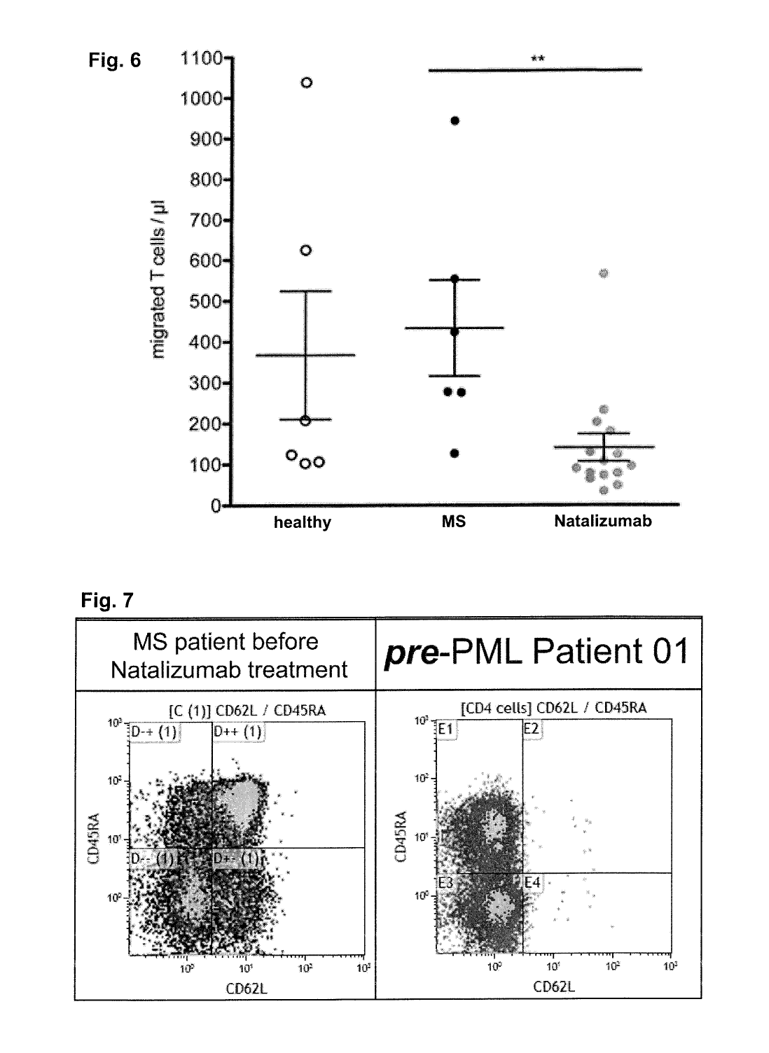

FIG. 6 shows the in vitro migration of isolated PBMC over primary human choroid plexus-derived epithelial cells (HCPEpiC). Given are the absolute values of migrated T cells per μl of sample represented by individual dots of healthy donors (open circles, n=6), untreated MS patients (black, n=6) or Natalizumab patients (grey, n=15). Migration has been assessed after 6 h.

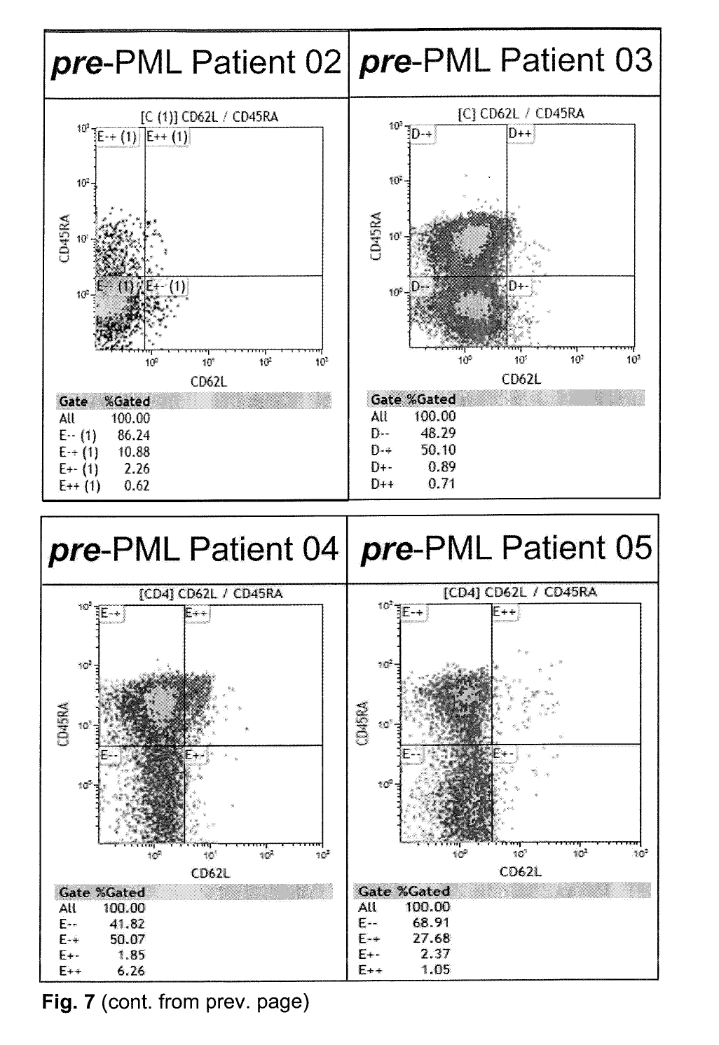

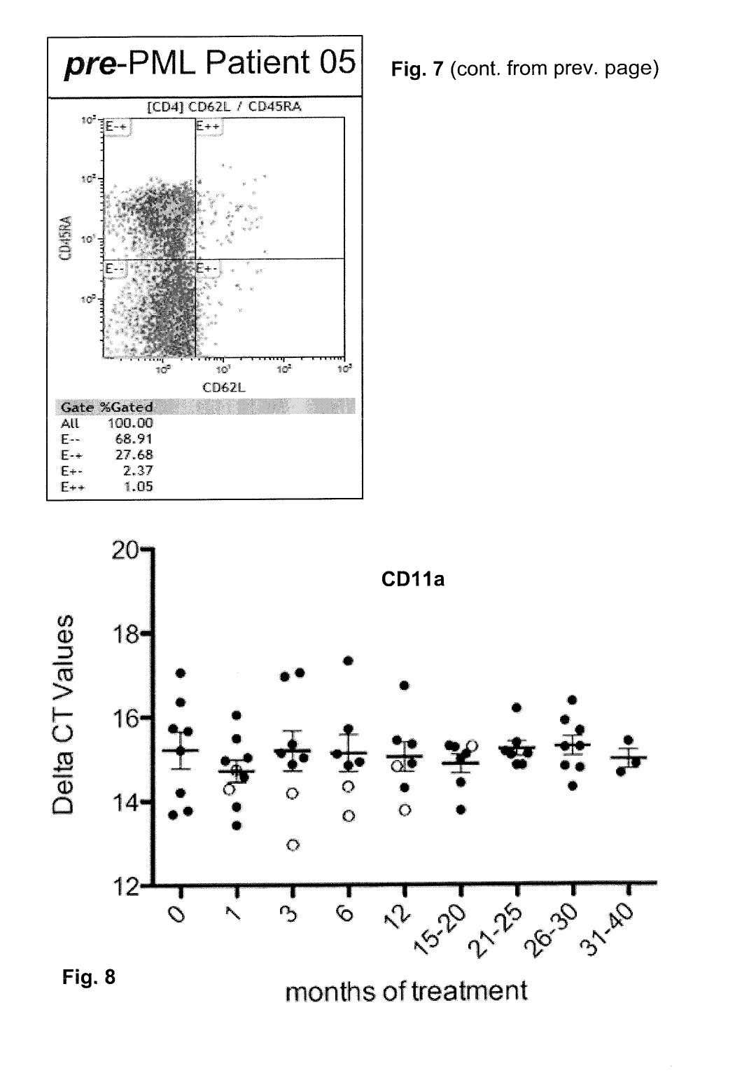

FIG. 7 depicts dot plots of samples of the six MS Natalizumab pre-PML patients and one exemplary MS patient before the start of Natalizumab therapy. The numbering of pre-PML patients is in line with the numbering used in FIG. 14. PBMC were first gated on “live lymphocytes”, then CD3+ (T cells), then CD4+ and finally plotted on CD62L vs. CD45RA to illustrate the loss of CD62L (especially striking on the CD45R+ (naive) CD4+ T cells).

FIG. 8 shows relative quantification of CD11a as compared to hS18 on thawed PBMC from MS patients before (month 0) and in the time course of therapy (months 1, 3, 6, 12, 15-20, 21-25, 26-30, 31-40, 41-50; n=27 patients) as assessed by real-time PCR. Lower delta CT values indicate a higher expression of the target.

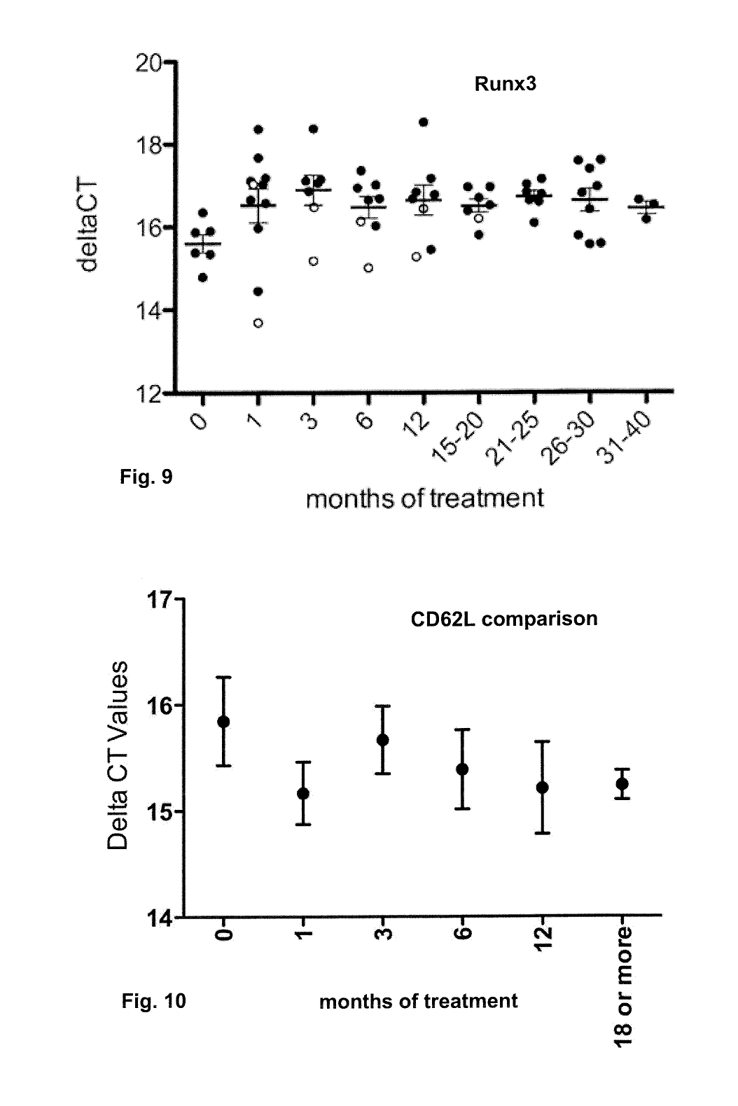

FIG. 9 shows relative quantification of Runs-3 as compared to hS18 on thawed PBMC from MS patients before (month 0) and in the time course of therapy (months 1, 3, 6, 12, 15-20, 21-25, 26-30, 31-40, 41-50; n=28 patients) as assessed by real-time PCR. Lower delta CT values indicate a higher expression of the target.

FIG. 10 shows relative quantification of CD62L as compared to hS18 on thawed PBMC from MS patients before (month 0) and in the time course of therapy (months 1, 3, 6, 12, or more; n=28 patients) as assessed by real-time PCR. Lower delta CT values indicate a higher expression of the target.

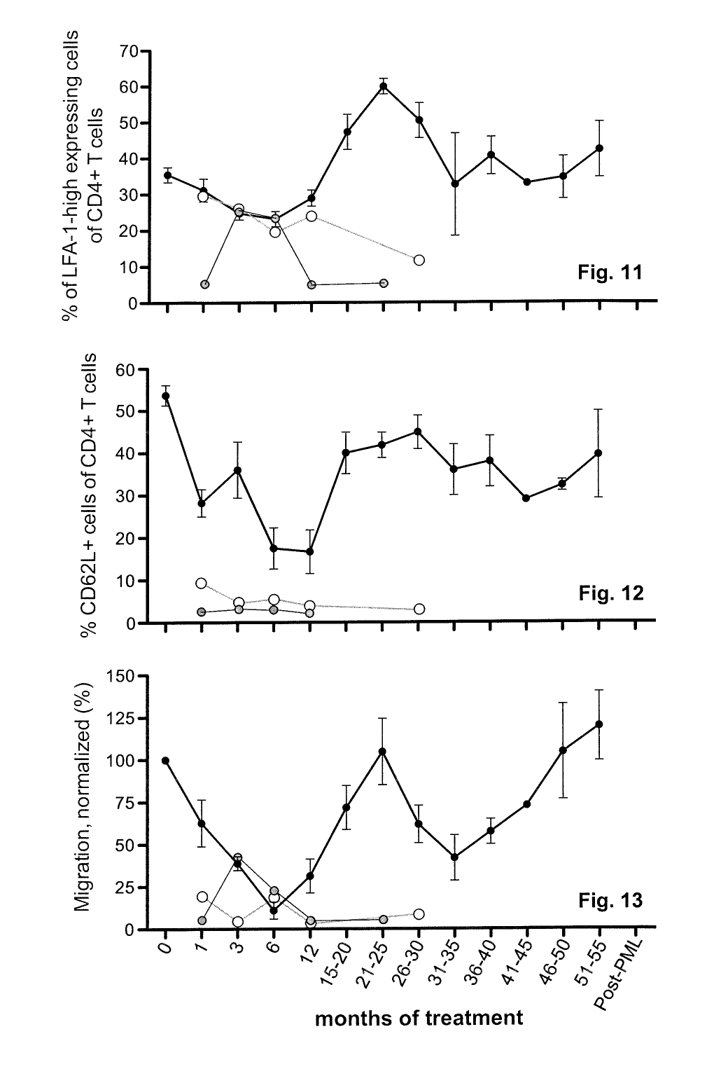

FIG. 11 shows the percentages of LFA-1 expressing T cells before Natalizumab treatment (month 0) and in the time course of therapy (months 1, 3, 6, 12, 15-20, 21-25, 26-30, 31-35, 36-40, 41-45, 46-50, 51-55; n=39 patients). Black symbols indicate the mean calculated from patients at given time points, standard error of the mean are given. The white and grey circles represent two patients who later developed PML.

FIG. 12 shows the percentages of CD62L expressing T cells before Natalizumab treatment (month 0) and in the time course of therapy (months 1, 3, 6, 12, 15-20, 21-25, 26-30, 31-35, 36-40, 41-45, 40-50, 51-55; n=39 patients). Black symbols indicate the mean calculated from patients at given time points, standard error of the mean are given. The white and grey circles represent two patients who later developed PML.

FIG. 13 shows the migration of CD3⁺ T cells (in percent, related to untreated MS patients set to 100%) before Natalizumab treatment (month 0) and in the time course of therapy (months 1, 3, 6, 12, 15-20, 21-25, 26-30, 31-35, 36-40, 41-45, 46-50, 51-55; n=50 patients). Black symbols indicate the mean calculated from patients at given time points, standard error means are given. The white and grey circles represent two patients who later developed PML.

FIG. 14 lists all patients included in this study. Given are cohort/patient, number of patients, year of birth, sex, first manifestation of MS, EDSS, pre-treatments, JCV antibody seropositivity, cycles of Natalizumab, % CD62L of CD4+ T cells (mean, standard deviation, and 10-90 percentile) of the following cohorts: Healthy controls, MS (naïve), MS (baseline treatments), MS (Natalizumab), MS (Natalizumab) pre-PML, MS (Natalizumab) acute-PML, MS (Natalizumab) post-PML, other monoclonal antibody-associated acute-PML and HIV-associated acute-PML, corresponding to the groups in FIG. 1A and FIG. 1B.

### DETAILED DESCRIPTION OF THE INVENTION

The present invention provides, amongst others, methods of determining a prognosis of the risk for PML occurrence. Using such a method according to the invention a subject can be identified as being at a higher risk of developing PML when compared to otherwise apparently similar subjects, e.g. subjects of comparable health/disease state or risk factor exposure. In some embodiments a respective method according to the invention can thus be taken to define a method of assessing the risk level of a subject with regard to PML occurrence. Based on such an assessment of the risk of occurrence of PML, a decision is in some embodiments taken as to whether a therapy, for example of administering an α₄-integrin-blocking agent and/or a VLA-4 blocking agent, or HAART is to be continued or discontinued. Methods of the invention also allow stratifying patients for risk of PML.

### DEFINITIONS

Unless otherwise stated, the following terms used in this document, including the description and claims, have the definitions given below.

The word “about” as used herein refers to a value being within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. The term “about” is also used to indicate that the amount or value in question may be the value designated or some other value that is approximately the same. The phrase is intended to convey that similar values promote equivalent results or effects according to the invention. In this context “about” may refer to a range above and/or below of up to 10%. The word “about” refers in some embodiments to a range above and below a certain value that is up to 5%, such as up to up to 2%, up to 1%, or up to 0.5% above or below that value. In one embodiment “about” refers to a range up to 0.1% above and below a given value.

The term “administering”, as used herein, refers to any mode of transferring, delivering, introducing, or transporting matter such as a compound, e.g. a pharmaceutical compound, or other agent such as an antigen, to a subject. Modes of administration include oral administration, topical contact, intravenous, intraperitoneal, intramuscular, intranasal, or subcutaneous administration (cf. below). Administration “in combination with” further matter such as one or more therapeutic agents includes simultaneous (concurrent) and consecutive administration in any order.

The term “antibody” generally refers to an immunoglobulin, a fragment thereof or a proteinaceous binding molecule with immunoglobulin-like functions (cf. below).

The word “assay” as used in this document refers to to a method, generally known in the art, to analyse a feature, e.g. a catalytic activity, the presence, the formation or the amount of matter occurring in a biological specimen. Such matter may be occurring in a living organism or representing a living organism, such as a protein, a nucleic acid, a lipid, a cell, a virus, a saccharide, a polysaccharide, a vitamin or an ion, to name a few examples. The word “assay” emphasizes that a certain procedure or series of procedures is followed, which may be taken to represent the respective assay. An assay may include quantitated reagents and established protocols to assess the presence, absence, amount or activity of a biological entity.

The term “binding assay” generally refers to a method of determining the interaction of matter. Hence, some embodiments of a binding assay can be used to qualitatively or quantitatively determine the ability of matter, e.g. a substance, to bind to other matter, e.g. a protein, a nucleic acid or any other substance. Some embodiments of a binding assay can be used to analyse the presence and/or the amount of matter on the basis of binding of the matter to a reagent such as a binding partner that is used in the method/assay. As two illustrative examples, a PSGL-1 binding assay or a CD62L binding assay may include the use of a binding partner such as an antibody (cf. below) that specifically binds to PSGL-1 and CD62L, respectively. Where a binding assay is based on the use of an immunoglobulin or a proteinaceous binding molecule with immunoglobulin-like functions as a binding partner such a method/procedure may also be called an “immunoassay”. In this regard, it is understood that the signals obtained from an immunoassay are a direct result of complexes formed between one or more immunoglobulins or proteinaceous binding molecules with immunoglobulin-like functions and the corresponding biomarker (i.e., the analyte) containing the necessary epitope(s) to which the binding partner(s) bind(s). While such an assay may detect the full length biomarker and the assay result be expressed as a concentration of a biomarker of interest, the signal from the assay is actually a result of all such “immunoreactive” molecules present in the sample. Expression of a biomarker may also be determined by means other than an immunoassay, including protein measurements such as dot blots, Western blots, chromatographic methods, mass spectrometry, and nucleic acid measurements such as mRNA quantification. For this purpose T cells may be isolated and optionally lysed, cf. also below.

A variety of methods for analysing binding of matter to other matter are known in the art. The techniques underlying such methods can for example be subdivided based on the use of a detectable label (cf. below). For example, some techniques require a labeled binding partner for signal detection, while others generate a signal based on the interaction of the analyte and the binding partner—including for instance measuring a mass change. Some techniques do not use labeled binding partners, but instead use a labeled analyte. Some techniques use two binding partners to create a an called “sandwich assay”, while others use only one binding partner (such as competitive assays). In sandwich assays, both binding partners bind specifically to the same analyte. In some embodiments, the two binding partners bind to differing portions, such as differing epitopes, of the analyte. Some techniques require a separation step to differentiate between a labeled binding partner that has bound an analyte and a labeled binding partner that has not bound an analyte. Some techniques do not require a separation step, such as agglutination assays and assays wherein the label on the labeled binding partner is modified, activated, or deactivated by the binding of the analyte. Some techniques require a support on which a binding partner is immobilized. A respective support may for instance be used in the context of a technique where two binding partners are employed—a first binding partner immobilized on the support, while a second binding partner is a labeled binding partner—to link the label to the support. By way of washing the support, any unbound, free labeled binding partner can then be removed prior to measuring the amount of label. The term “chemotaxis assay” as used herein refers to a method established in the art that can be used to measure the migration of certain cells in a given environment.

The term “binding partner” as used herein refers to matter, such as a molecule, in particular a polymeric molecule, that can bind a nucleic acid molecule such as a DNA or an RNA molecule, including an mRNA molecule, as well as a peptide, a protein, a saccharide, a polysaccharide or a lipid through an interaction that is sufficient to permit the agent to form a complex with the nucleic acid molecule, peptide, protein or saccharide, a polysaccharide or a lipid, generally via non-covalent bonding. In some embodiments the binding partner is a PNA molecule. In some embodiments the binding partner is an immunoglobulin or a proteinaceous binding molecule with immunoglobulin-like functions as defined below. In some embodiments the binding partner is an aptamer. In some embodiments a binding partner is specific for a particular target. In some embodiments a binding partner includes a plurality of binding sites, each binding site being specific for a particular target. As an illustrative example, a binding partner may be a proteinaceous agent with immunoglobulin-like functions with two binding sites. It may for instance be a bispecific diabody, such as a bispecific single chain diabody.

The term “biomarker” as used herein refers to a protein or a gene encoding the protein, which is expressed at a lower level in, or found at a lower level on, T cells of individuals that are at risk as compared to not at risk of PML occurrence.

The term “detect” or “detecting”, as well as the term “determine” or “determining” when used in the context of a biomarker, refers to any method that can be used to detect the presence of a nucleic acid (DNA and RNA) or a protein/polypeptide. When used herein in combination with the words “level”, “amount” or “value”, the words “detect”, “detecting”, “determine” or “determining” are understood to generally refer to a quantitative rather than a qualitative level. Accordingly, a method according to the invention includes a quantification of CD62L, PSGL-1 and/or LFA-1—i.e. the amount or number of CD62L expressing, PSGL-1 expressing and/or LFA-1 expressing T cells, e.g. CD3 positive T cells, is analysed. In this regard the words “value,” “amount” and “level” are used interchangeably herein. The terms “value,” “amount” and “level” also refer to the rate of synthesis of CD62L, PSGL-1 and/or LFA-1 in CD3⁺ T cells, as explained further below. The exact nature of the “level”, “amount” or “value” depends on the specific design and components of the particular analytical method employed to detect CD62L, PSGL-1 and/or LFA-1 or other biomarker.

The term “detectable label” is used to herein to refer to any substance the detection or measurement of which, either directly or indirectly, by physical or chemical means, is indicative of the presence of a selected target bioentity in a sample. Representative examples of useful detectable labels include, but are not limited to, molecules or ions directly or indirectly detectable based on light absorbance, fluorescence, reflectivity, light scatter, phosphorescence, or luminescence properties, molecules or ions detectable by their radioactive properties or molecules or ions detectable by their nuclear magnetic resonance or paramagnetic properties. A detectable label may in some embodiments be a molecule that can be indirectly detected based on light absorbance or fluorescence, for example, various enzymes which cause appropriate substrates to convert, e.g., from non-light absorbing to light absorbing molecules, or from non-fluorescent to fluorescent molecules.

A “differential”, “differing” or “altered” expression, as used throughout the present application, is observed when a difference in the level of expression of a biomarker of the invention can be analysed by measuring the level of expression of the products of the biomarkers of the invention, such as the difference in level of RNA expressed, the difference of the amount on cells or the difference of cells carrying the biomarker on their cell surface. A differential expression is for example observed when the expression of a protein, e.g. on the surface of a cell, is lower or higher than that observed from one or more control subjects such that one of skill in the art would consider it to be of statistical significance. As further explained below, in some embodiments the expression/amount of a protein is considered differential or altered when gene expression/amount is increased or decreased by about 10% as compared to the control level. The expression/amount of a protein is in some embodiments considered differential when it is increased or decreased by about 25% when compared to the control level. In some embodiments the expression/amount of a protein is considered altered when gene expression/amount is increased or decreased by about 50%. In some embodiments the expression/amount of a protein is considered differential when it is increased or decreased by about 75%, including about 100%, or higher, as compared to the control level. In some embodiments an expression level or an amount is deemed “differential”, “increased” or “decreased” when gene expression/amount is increased or decreased by at least about 0.1 fold, as compared to a control level. In some embodiments an expression level or an amount is considered differential when it is increased or decreased by at least about 0.2 fold. In some embodiments the expression/amount of a protein is considered differential when it is increased or decreased by about a factor of 1, including at least about 2. In some embodiments an expression level or an amount is deemed “differential” when gene expression amount is increased or decreased by at least about 5 fold, as compared to a control level.

Generally a biomarker of the present invention is expressed at a lower level when a subject is at an increased risk of PML occurrence. The term “differential”, “differing” or “altered” expression can also refer to an increase or decrease in the measurable expression level of a given biomarker in a population of cells as compared with the measurable expression level of a biomarker in a second population of cells. In one embodiment, the differential expression can be compared using the ratio of the level of expression of a given biomarker or biomarkers as compared with the expression level of the given biomarker or biomarkers of a control as further explained below. A differential expression means that the respective ratio is not equal to 1.0. For example, an RNA is differentially expressed if the ratio of the level of expression in a first sample as compared with a second sample is greater than or less than 1.0. For example, a ratio of greater than 1 or than 1.2 is an expression differing from the reference. As a further example, where the ratio of expression between a first and a second sample is about 1.5 or more the expression is altered or different. In some embodiments an expression with a ratio of about 1.7 or greater is regarded as altered or different. In some embodiments a ratio of expression levels of about 2, 3, 3, 5, 10, 15, 20 or more is taken to be altered or differential/different. As a further example, where the ratio of expression between samples is less than 1 or about 0.8 or less the expression is altered or different. In some embodiments expression levels are regarded as different/altered when the ratio is 0.6 or less. In some embodiments a ratio of expression levels of about 0.6, 0.4, 0.2, 0.1, 0.05, 0.001 or less is taken to be different. In some embodiments the differential expression is measured using p-value. For instance, when using p-value, a biomarker is identified as being differentially expressed as between a first and second population when the p-value is less than about 0.1, including less than about 0.05. In some embodiments expression levels are regarded as different/altered when the p-value is less than about 0.01. In some embodiments expression levels that have a p-value of less than about 0.005 are regarded as different/altered. In some embodiments expression levels are regarded as different/altered when the p-value is less than about 0.001.

An “effective amount” or a “therapeutically effective amount” of a compound, such as an anti-retroviral compound, an α₄-integrin blocking agent or a VLA-4 blocking agent, is an amount—either as a single dose or as part of a series of doses—sufficient to provide a therapeutic benefit in the treatment or management of the relevant pathological condition, or to delay or minimize one or more symptoms associated with the presence of the condition. Such a condition may be associated with immunosuppression, e.g. an autoimmune disease, or with a retroviral infection.

An “epitope” is antigenic and thus an epitope may also be taken to define an “antigenic structure” or “antigenic determinant”. Thus, a binding domain of an immunoglobulin or of a proteinaceous binding molecule with immunoglobulin-like functions is an “antigen-interaction-site”. The term “antigen-interaction-site” defines, in accordance with the present invention, a motif of a polypeptide, which is able to specifically interact with a specific antigen or a specific group of antigens, e.g. L-selectin, PSGL-1 and/or LFA-1 in different species. This binding/interaction is also understood to define a “specific recognition”. An epitope usually consists of spatially accessible surface groupings of moieties of one or more chemical entities such as polypeptide chains or mono- or polysaccharides. Surface groupings defining an epitope may for instance be groupings of amino acids or sugar side chains. An epitope usually has specific three dimensional structural characteristics, as well as specific charge characteristics. Conformational and nonconformational epitopes are distinguished in that the binding to the former but not the latter is lost in the presence of denaturing solvents (cf. also below).

The term “epitope” also refers to a site on an antigen such as CD3, CD4 or CD8, with which an immunoglobulin, a T cell receptor or a proteinaceous binding molecule with immunoglobulin-like functions forms a complex. In some embodiments, an epitope is a site on a molecule against which an immunoglobulin or a proteinaceous binding molecule with immunoglobulin-like functions will be produced and/or to which an antibody will bind. For example, an epitope can be recognized by an immunoglobulin or a proteinaceous binding molecule with immunoglobulin-like functions. The epitope may be a “linear epitope”, which is an epitope where an amino acid primary sequence contains the epitope recognized. A linear epitope typically includes at least 3, and more usually, at least 5 amino acids in a unique sequence. A linear epitope may for example include about 8 to about 10 amino acids in a unique sequence. The epitope may also be a “conformational epitope”, which in contrast to a linear epitope, is an epitope where the primary sequence of the amino acids that includes the epitope is not the sole defining component of the epitope recognized (e.g., an epitope wherein the primary sequence of amino acids is not necessarily recognized by the antibody defining the epitope). Typically a conformational epitope includes a larger number of amino acids than a linear epitope. With regard to recognition of conformational epitopes, an immunoglobulin or a proteinaceous binding molecule with immunoglobulin-like functions recognizes a 3-dimensional structure of the antigen, such as a peptide or protein, or a fragment of a peptide or protein. As an illustrative example, when a protein molecule folds to form a three dimensional structure, certain amino acids and/or all or portions of the polypeptide backbone forming the conformational epitope become juxtaposed, allowing an antibody to recognize the epitope. Methods of determining conformation of epitopes include, but are not limited to, x-ray crystallography, 2-dimensional nuclear magnetic resonance spectroscopy, site-directed spin labeling and electron paramagnetic resonance spectroscopy.

By the use of the term “enriched” in reference to a polypeptide, a nucleic acid or a cell is meant that the specific amino acid/nucleotide sequence or cell, including cell population, constitutes a significantly higher fraction (2-5 fold) of the total amino acid sequences or nucleic acid sequence present in the sample of interest than in the natural source from which the sample was obtained. The polypeptide, a nucleic acid or a cell may also constitute a significantly higher fraction than in a normal or diseased organism or than in normal or diseased cells or in the cells from which the sequence was taken. This could be caused by preferential reduction in the amount of other amino acid/nucleotide sequences or cells present, or by a preferential increase in the amount of the specific amino acid/nucleotide sequence or cell of interest, or by a combination of the two. However, it should be noted that enriched does not imply that there are no other amino acid sequences, nucleotide sequences or cells present. The term merely defines that the relative amount of the sequence of interest has been significantly increased. The term significant here is used to indicate that the level of increase is useful to the person achieving such an increase, and generally means an increase relative to other amino acid or nucleic acid sequences of about at least 2-fold, for example at least about 5- to 10-fold or even more. The term is meant to cover only those situations in which man has intervened to increase the proportion of the desired amino acid sequence, nucleotide sequence or cell.

The term “essentially consists of” is understood to allow the presence of additional components in a sample or a composition that do not affect the properties of the sample or a composition. As an illustrative example, a pharmaceutical composition may include excipients if it essentially consists of an active ingredient.

The terms “expressing” and “expression” in reference to a biomarker are intended to be understood in the ordinary meaning as used in the art. A biomarker is expressed by a cell via transcription of a nucleic acid into mRNA, followed by translation into a polypeptide, which is folded and possibly further processed. The biomarkers discussed in this disclosure are in addition being transported to the surface of the respective cell. Hence, the statement that a cell is expressing such a biomarker indicates that the biomarker is found on the surface of the cell and implies that the biomarker has been synthesized by the expression machinery of the respective cell. Accordingly, the term “expression level” in the context of a cell population such as T cells refers to the number or percentage of cells that have the biomarker of interest on their cell surface. The determination of expression may be based on the normalized expression level of the biomarkers. Expression levels are normalized by correcting the absolute expression level of a biomarker by comparing its expression to the expression of a gene that is not a biomarker in the context of the invention. The expression level may also be provided as a relative expression level.

With regard to the respective biological process itself, the terms “expression”, “gene expression” or “expressing” refer to the entirety of regulatory pathways converting the information encoded in the nucleic acid sequence of a gene first into messenger RNA (mRNA) and then to a protein. Accordingly, the expression of a gene includes its transcription into a primary hnRNA, the processing of this hnRNA into a mature RNA and the translation of the mRNA sequence into the corresponding amino acid sequence of the protein. In this context, it is also noted that the term “gene product” refers not only to a protein, including e.g. a final protein (including a splice variant thereof) encoded by that gene and a respective precursor protein where applicable, but also to the respective mRNA, which may be regarded as the “first gene product” during the course of gene expression.

By “fragment” in reference to a polypeptide such as an immunoglobulin or a proteinaceous binding molecule is meant any amino acid sequence present in a corresponding polypeptide, as long as it is shorter than the full length sequence and as long as it is capable of performing the function of interest of the protein—in the case of an immunoglobulin specifically binding to the desired target, e.g. antigen (CD62L, LFA-1 or PSGL-1, for example). The term “immunoglobulin fragment” refers to a portion of an immunoglobulin, often the hypervariable region and portions of the surrounding heavy and light chains that displays specific binding affinity for a particular molecule. A hypervariable region is a portion of an immunoglobulin that physically binds to the polypeptide target.

The terms “immunize”, “immunization”, or “immunizing” refer to exposing the immune system of an animal to an antigen or to an epitope thereof as illustrated in more detail below. The antigen may be introduced into the animal using a desired route of administration, such as injection, inhalation or ingestion. Upon a second exposure to the same antigen, the adaptive immune response, in particular T cell and B cell responses, is enhanced.

The term “isolated” indicates that the cell or cells, or the peptide(s) or nucleic acid molecule(s) has/have been removed from its/their normal physiological environment, e.g. a natural source, or that a peptide or nucleic acid is synthesized. Use of the term “isolated” indicates that a naturally occurring sequence has been removed from its normal cellular (i.e., chromosomal) environment. Thus, the sequence may be in a cell-free solution or placed in a different cellular environment. An isolated cell or isolated cells may for instance be included in a different medium such as an aqueous solution than provided originally, or placed in a different physiological environment. Typically isolated cells, peptides or nucleic acid molecule(s) constitute a higher fraction of the total cells, peptides or nucleic acid molecule(s) present in their environment, e.g. solution/suspension as applicable, than in the environment from which they were taken. By “isolated” in reference to a polypeptide or nucleic acid molecule is meant a polymer of amino acids (2 or more amino acids) or nucleotides coupled to each other, including a polypeptide or nucleic acid molecule that is isolated from a natural source or that is synthesized. The term “isolated” does not imply that the sequence is the only amino acid chain or nucleotide chain present, but that it is essentially free, e.g. about 90-95% pure or more, of e.g. non-amino acid material and/or non-nucleic acid material, respectively, naturally associated with it.

Isolation of a desired population of cells may in some embodiments include general cell enrichment techniques such as centrifugation, filtration or cell chromatography. Generally, isolating or enriching a desired population of cells may be carried out according to any desired technique known in the art. In some embodiments isolation of a desired population of cells may include the use of a commercially available cell isolation kit. T cells may for instance be obtained from peripheral blood, from blood, cerebrospinal fluid, or enriched fractions thereof. T cells may for instance be obtained from peripheral blood mononuclear cells (PBMC) such as human PBMCs. In some embodiments PBMC may for instance be enriched using a standard technique based on cell density and/or cell size. As an illustrative example, PBMC may be enriched or isolated via density gradient centrifugation, for example using sucrose, dextran, Ficoll® or Percoll®. T cells may then be enriched or purified from the obtained PBMCs, for example using a commercially available T cell isolation kit such as the Dynabeads Untouched™ Human CD4 T Cells kit available from Invitrogen or the StemSep® Human CD4+ T Cell Enrichment Kit from STEMCELL Technologies Inc.

The term “nucleic acid molecule” as used herein refers to any nucleic acid in any possible configuration, such as single stranded, double stranded or a combination thereof. Examples of nucleic acids include for instance DNA molecules, RNA molecules, analogues of the DNA or RNA generated using nucleotide analogues or using nucleic acid chemistry, locked nucleic acid molecules (LNA), protein nucleic acids molecules (PNA), alkylphosphonate and alkylphosphotriester nucleic acid molecules and tecto-RNA molecules (e.g. Liu, B., et al., J. Am. Chem. Soc. (2004) 126, 4076-4077). LNA has a modified RNA backbone with a methylene bridge between C4′ and O2′, providing the respective molecule with a higher duplex stability and nuclease resistance. Alkylphosphonate and alkylphosphotriester nucleic acid molecules can be viewed as a DNA or an RNA molecule, in which phosphate groups of the nucleic acid backbone are neutralized by exchanging the P—OH groups of the phosphate groups in the nucleic acid backbone to an alkyl and to an alkoxy group, respectively. DNA or RNA may be of genomic or synthetic origin and may be single or double stranded. Such nucleic acid can be e.g. mRNA, cRNA, synthetic RNA, genomic DNA, cDNA synthetic DNA, a copolymer of DNA and RNA, oligonucleotides, etc. A respective nucleic acid may furthermore contain non-natural nucleotide analogues and/or be linked to an affinity tag or a label.

Many nucleotide analogues are known and can be used in nucleic acids used in the methods of the invention. A nucleotide analogue is a nucleotide containing a modification at for instance the base, sugar, or phosphate moieties. As an illustrative example, a substitution of 2′-OH residues of siRNA with 2′F, 2′O-Me or 2′H residues is known to improve the in vivo stability of the respective RNA. Modifications at the base moiety may be a natural or a synthetic modification of A, C, G, and T/U, a different purine or pyrimidine base, such as uracil-5-yl, hypoxanthin-9-yl, and 2-aminoadenin-9-yl, as well as a non-purine or a non-pyrimidine nucleotide base. Other nucleotide analogues serve as universal bases. Examples of universal bases include 3-nitropyrrole and 5-nitroindole. Universal bases are able to form a base pair with any other base. Base modifications often can be combined with for example a sugar modification, such as for instance 2′-β-methoxyethyl, e.g. to achieve unique properties such as increased duplex stability.

The term “occurrence of PML” as used in this disclosure includes a condition having one or more characteristics indicative of the presence of PML. As explained above, the typical characteristic of PML is the demyelination in brain. The characteristic of PML generally used in the art for diagnostic purposes is the presence of JCV DNA in cerebrospinal fluid or a brain biopsy specimen (cf. also below). Further characteristics may be assessed, e.g. visual field testing, ophthalmologic examination and/or cranial magnetic resonance imaging may be performed.

As used in this document, the expression “pharmaceutically acceptable” refers to those active compounds, materials, compositions, carriers, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

“Plasma” as used in this disclosure refers to acellular fluid found in blood. “Plasma” may be obtained from blood by removing whole cellular material from blood by methods known in the art such as centrifugation or filtration.

The terms “polypeptide” and “protein” refer to a polymer of amino acid residues and are not limited to a certain minimum length of the product. Where both terms are used concurrently, this twofold naming accounts for the use of both terms side by side in the art.

The term “predicting the risk” as used in the disclosure refers to assessing the probability that a subject will suffer from PML in the future. As will be understood by those skilled in the art, such an assessment is usually not intended to be correct for 100% of the subjects to be investigated. The term, however, requires that a prediction can be made for a statistically significant portion of subjects in a proper and correct manner. Whether a portion is statistically significant can be determined by those skilled in the art using various well known statistic evaluation tools, e.g., determination of confidence intervals, p-value determination, Student's t-test, and Mann-Whitney test. Suitable confidence intervals are generally at least 90%, at least 95%, at least 97%, at least 98%, or at least 99%. Suitable p-values are generally 0.1, 0.05, 0.01, 0.005, or 0.0001. In one embodiment of the disclosed methods, the probability envisaged by the present disclosure allows that the prediction of an increased, normal, or decreased risk will be correct for at least 60%, at least 70%, at least 80%, or at least 90% of the subjects of a given cohort or population. Predictions of risk in a disclosed method relates to predicting whether or not there is an increased risk for PML compared to the average risk for developing PML in a population of subjects rather than giving a precise probability for the risk.

In this regard the term “prognosis”, commonplace and well-understood in medical and clinical practice, refers to a forecast, a prediction, an advance declaration, or foretelling of the probability of occurrence of a disease state or condition in a subject not (yet) having the respective disease state or condition. In the context of the present invention prognosis refers to the forecast or prediction of the probability as to whether a subject will or will not suffer from PML.

The term “preventing” in the medical/physiological context, i.e. in the context of a physiological state, refers to decreasing the probability that an organism contracts or develops an abnormal condition.

The term “purified” is understood to be a relative indication in comparison to the original environment of the cell, thereby representing an indication that the cell is relatively purer than in the natural environment. It therefore includes, but does not only refer to, an absolute value in the sense of absolute purity from other cells (such as a homogeneous cell population). Compared to the natural level, the level after purifying the cell will generally be at least 2-5 fold greater (e.g., in terms of cells/ml). Purification of at least one order of magnitude, such as about two or three orders, including for example about four or five orders of magnitude is expressly contemplated. It may be desired to obtain the cell at least essentially free of contamination, in particular free of other cells, at a functionally significant level, for example about 90%, about 95%, or 99% pure. With regard to a nucleic acid, peptide or a protein, the above applies mutatis mutandis. In this case purifying the nucleic acid, peptide or protein will for instance generally be at least 2-5 fold greater (e.g., in terms of mg/ml).

When used in the context of expression of a biomarker, e.g. PSGL-1 or CD62L, on cells and the amount or level of a biomarker that can be detected, “recovery” is defined as an increase in the amount/level following a decrease. A recovery of expression may be a return of the level of the biomarker to a level that has previously been observed for a given subject, or to a higher level. Generally a recovery is a return of the percentage of cells which express the biomarker back to the range of a reference level or higher. A recovery is determined by comparing a determined amount or level to a threshold value, which may be based on a reference level (cf. below).

Diagnosing, determining, assessing or predicting the “risk of occurrence” of PML is understood to refer to an analysis of a relative degree of a risk when compared to a healthy individual. The term “risk of occurrence” refers to the likelihood or probability that PML will occur in a subject. Without being bound by theory, PML is thought to be a reactivation of latent infection (cf. above) with JCV. While a general susceptibility to occurrence of PML is linked to the presence of JCV in a subject's organism, the mere presence of JCV in an organism does not indicate whether there is or will be an infection of oligodendrocytes with JCV, which leads to demyelination (supra). Hence, there can generally only be an elevated risk of PML occurrence if JCV is present in an organism, however, the actual risk level needs to be determined on the basis of the level of PSGL-1, CD62L and/or LFA-1. In the context of diagnosis and risk assessment, determining/predicting the risk of occurrence of PML is a relative assessment whether a particular subject is at a higher risk or not at a higher risk of suffering from PML at a point of time in the future, when compared to a healthy subject or to an average subject that is in an otherwise comparable physiological condition.

The word “recombinant” is used in this document to describe a nucleic acid molecule that, by virtue of its origin, manipulation, or both is not associated with all or a portion of the nucleic acid molecule with which it is associated in nature. Generally a recombinant nucleic acid molecule includes a sequence which does not naturally occur in the respective wildtype organism or cell.

Typically a recombinant nucleic acid molecule is obtained by genetic engineering, usually constructed outside of a cell. Generally a recombinant nucleic acid molecule is substantially identical and/or substantial complementary to at least a portion of the corresponding nucleic acid molecule occurring in nature. A recombinant nucleic acid molecule may be of any origin, such as genomic, cDNA, mammalian, bacterial, viral, semisynthetic or synthetic origin The term “recombinant” as used with respect to a protein/polypeptide means a polypeptide produced by expression of a recombinant polynucleotide.

The term “reducing the risk”, as used in this document, means to lower the likelihood or probability of a disease state or condition, e.g., PML, from occurring in a subject, especially when the subject is predisposed to such or at risk of contracting a disease state or condition, e.g., PML.

The terms “screening subjects”, “screening individuals” or “screening patients” in the context of risk assessment refers to a method or process of determining if a subject/patient or a plurality of subjects/patients is or is not likely to suffer from a disease or disorder such as PML, or has or does not have an increased risk of developing a disease or disorder. “Screening compounds” and a “screening assay” means a process or method used to characterize or select compounds based upon their activity from a collection of compounds.

“Serum” as used in this disclosure, refers to components of blood that do not define a cell, such as a leukocyte, and that do not define a clotting factor. Serum includes the fraction of plasma obtained after plasma or blood is permitted to clot and the clotted fraction is removed.

The term “specific” as used in this document is understood to indicate that a binding partner is directed against, binds to, or reacts with a biomarker disclosed in the present application, such as PSGL-1, LFA-1, CD4, CD8, CD62L and CD3. Thus, being directed to, binding to or reacting with includes that the binding partner specifically binds to CD62L, LFA-1, PSGL-1, CD4, CD8 or CD3, as applicable. The term “specifically” in this context means that the binding partner reacts with CD62L, LFA-1, PSGL-1, CD4, CD8 or CD3, as applicable, or/and a portion thereof, but at least essentially not with another protein. The term “another protein” includes any protein, including proteins closely related to or being homologous to e.g. CD62L, PSGL-1, LFA-1 or CD3 against which the binding partner is directed to. The term “does not essentially bind” means that the binding partner does not have particular affinity to another protein, i.e., shows a cross-reactivity of less than about 30%, when compared to the affinity to CD62L, LFA-1, PSGL-1 or CD3. In some embodiments the binding partner shows a cross-reactivity of less than about 20%, such as less than about 10%. In some embodiments the binding partner shows a cross-reactivity of less than about 9, 8, or 7%, when compared to the affinity to CD62L, LFA-1, PSGL-1 or CD3. In some embodiments the binding partner shows a cross-reactivity of less than about 6%, such as less than about 5%, when compared to the affinity to CD62L, LFA-1, PSGL-1 or CD3. Whether the binding partner specifically reacts as defined herein above can easily be tested, inter alia, by comparing the reaction of a respective binding partner with CD62L, with LFA-1, PSGL-1 or with CD3, as applicable, and the reaction of the binding partner with (an) other protein(s). The term “specifically recognizing”, which can be used interchangeably with the terms “directed to” or “reacting with” means in the context of the present disclosure that a particular molecule, generally an immunoglobulin, an immunoglobulin fragment or a proteinaceous binding molecule with immunoglobulin-like functions is capable of specifically interacting with and/or binding to at least two, including at least three, such as at least four or even more amino acids of an epitope as defined herein. Generally the immunoglobulin or proteinaceous binding molecule can thereby form a complex with the respective epitope of e.g. CD62L, LFA-1, PSGL-1 or CD3. Such binding may be exemplified by the specificity of a “lock-and-key-principle”. “Specific binding” can also be determined, for example, in accordance with a Western blot, ELISA-, RIA-, ECL-, IRMA-test, FACS, IHC and a peptide scan.

The terms “stratifying” and “stratification” as used herein indicate in one aspect that individuals are assigned to groups with similar characteristics such as at a similar risk level of developing PML. As an illustrative example, individuals may be stratified into risk categories. The terms “stratifying” and “stratification” as used herein indicate in another aspect that an individual is assigned to a certain group according to characteristics matching the respective group such as a corresponding risk level of developing PML. The groups may be, for example, for testing, prescribing, suspending or abandoning any one or more of a drug, surgery, diet, exercise, or intervention. Accordingly, in some embodiments of a method or use according to the invention a subject may be stratified into a subgroup of a clinical trial of a therapy. As explained in the following, in the context of the present invention CD62L, PSGL-1 and/or LFA-1 may be used for PML risk stratification.

The terms “stratifying” and “stratification” according to the invention generally include identifying subjects that require an alteration of their current or future therapy. The term includes assessing, e.g. determining, which therapy a subject likely to suffer from PML is in need of. Hence, in the context of the present invention stratification may be based on the probability (or risk) of developing PML. A method or use according to the invention may also serve in stratifying the probability of the risk of PML or the risk of any PML related condition for a subject. A method of stratifying a subject for PML therapy according to the invention includes detecting the amount of determining the expression level of CD62L, PSGL-1 and/or LFA-1 as described above, and/or assessing the migratory capacity of CD45+CD49d+ immune cells of the subject. As explained above, in some embodiments on a general basis a CD62L, a PSGL-1 and/or a LFA-1 binding partner can be advantageously used to screen risk patients which are at a higher risk or have a higher predisposition to develop PML.

The term “subject” as used herein, also addressed as an individual, refers to a human or non-human animal, generally a mammal. A subject may be a mammalian species such as a rabbit, a mouse, a rat, a Guinea pig, a hamster, a dog, a cat, a pig, a cow, a goat, a sheep, a horse, a monkey, an ape or a human. Thus, the methods, uses and compositions described in this document are applicable to both human and veterinary disease. As explained in more detail below, the sample has been obtained from the subject. It is thus understood that conclusions drawn from expression levels in the sample and decisions based thereon concern the subject from whom/which the subject has been taken. Further, while a subject is typically a living organism, the invention described in this document may also be used in post-mortem analysis. Where the subject is a living human who is receiving medical care for a disease or condition, it is also addressed as a “patient”.

The term “susceptibility” as used in this document refers to the proneness of a subject towards the development of a certain state or a certain condition such as a pathological condition, including a disease or disorder, in particular PML, or towards being less able to resist a particular state than the average individual. Susceptibility to PML is in particular dependent on the presence of JCV in an organism.

The terms “treatment” and “treating” as used herein, refer to a prophylactic or preventative measure having a therapeutic effect and preventing, slowing down (lessen), or at least partially alleviating or abrogating an abnormal, including pathologic, condition in the organism of a subject. Those in need of treatment include those already with the disorder as well as those prone to having the disorder or those in whom the disorder is to be prevented (prophylaxis). Generally a treatment reduces, stabilizes, or inhibits progression of a symptom that is associated with the presence and/or progression of a disease or pathological condition. The term “administering” relates to a method of incorporating a compound into cells or tissues of a subject. The term “therapeutic effect” refers to the inhibition or activation of factors causing or contributing to the abnormal condition. A therapeutic effect relieves to some extent one or more of the symptoms of an abnormal condition or disease. The term “abnormal condition” refers to a function in the cells or tissues of an organism that deviates from their normal functions in that organism. An abnormal condition can inter alia relate to cell proliferation, cell differentiation, or cell survival.

As used herein, the term “viable” refers to a cell that maintains homeostasis by the use of one or more energy consuming mechanisms. Thus a “viable” cell for example includes a cell in which productive oxidative metabolism occurs to produce the necessary energy; a cell in which only glycolysis is used to produce energy, as well as a cell which maintains cellular integrity, such as the ability to exclude, or actively remove, certain molecules from the interior of the cell, by energy consuming mechanisms. In some embodiments, a viable cell is capable of undergoing mitosis, cell growth, differentiation, and/or proliferation. The expression “viable cell” can be taken to be synonymous with a “living cell”, which includes a cell that is quiescent (and thus not going through the cell cycle), but nonetheless alive because energy production and consumption occurs in such a cell to maintain homeostasis.

The term “VLA-4 blocking agent” refers to a molecule that binds to the VLA-4 antigen on the surface of a leukocyte with sufficient specificity to inhibit the VLA-4/VCAM-1 interaction. In some embodiments the blocking agent binds to VLA-4 integrin with a K_{D} of less than 10⁻⁶ M. A VLA-4 blocking agent may be a VLA-4 binding antibody such as an anti-VLA-4 immunoglobulin or a fragment of an anti-VLA-4 immunoglobulin (cf. below for details). A VLA-4 blocking agent generally inhibits the migration of leukocytes from the blood to the central nervous system by disrupting adhesion between the T-cell and endothelial cells. This is believed to result in the reduction of proinflammatory cytokines, and thus the reduction of the occurrence of pathologic inflammatory disease within the CNS. Examples of a VLA-4 blocking agent include, but are not limited to, Natalizumab (Biogen, U.S. Pat. No. 5,840,299), monoclonal immunoglobulins HP2/1, HP1/3 (Elices et al, Cell (1990) 60, 577-584), HP1/2 (Sanchez-Madrid et al, Eur. J. Immunol. (1986) 16, 1343-1349), humanized HP1/2 (U.S. Pat. No. 6,602,503), HP1/7, HP2/4, B-5G10, TS2/16 (Pulido et al, J. Biol. Chem. (1991) 266, 16, 10241-5), monoclonal immunoglobulin L25 (Becton Dickinson GmBH, Germany), P4C2 (Abcam, Cambridge, UK), and AJM300 (Ajinomoto, Japan), and recombinant anti-VLA4 immunoglobulins as described in U.S. Pat. No. 6,602,503 and U.S. Pat. No. 7,829,092. In one embodiment, the VLA-blocking agent is specific for CD49d (α4-integrin). As a further example, a VLA-4 blocking agent may also be a VLA-4 antagonist that differs from an antibody such as an immunoglobulin. Illustrative example of such an antagonist are the low molecular weight compound SB-683699 (GlaxoSmithKline, Middlesex, UK), which is a dual (α4 antagonist, a CS-1 peptidomimetic (U.S. Pat. Nos. 5,821,231, 5,869,448, 5,869,448; 5,936,065; 6,265,572; 6,288,267; 6,365,619; 6,423,728; 6,426,348; 6,458,844; 6,479,666; 6,482,849; 6,596,752; 6,667,331; 6,668,527; 6,685,617; 6,903,128; and 7,015,216), a phenylalanine derivative (U.S. Pat. Nos. 6,197,794; 6,229,011; 6,329,372; 6,388,084; 6,348,463; 6,362,204; 6,380,387; 6,445,550; 6,806,365; 6,835,738; 6,855,706; 6,872,719; 6,878,718; 6,911,451; 6,916,933; 7,105,520; 7,153,963; 7,160,874; 7,193,108; 7,250,516; and 7,291,645) alphafeto protein (U.S. Pat. Pub. No. 2010/0150915), a beta-amino acid compound (U.S. Pat. Pub. Nos. 2004/0229859 and 2006/0211630), a semi-peptide compound (U.S. Pat. No. 6,376,538), Leu-Asp-Val tripeptide (U.S. Pat. No. 6,552,216), or a pegylated molecule as described in U.S. patent application 2007/066533 and U.S. Pat. No. 6,235,711.

An “α₄-integrin blocking agent” refers to a molecule that binds to the α₄-subunit of integrins with a specificity and an affinity and/or k_{off} rate that is sufficient to inhibit the interaction with a physiological ligand such as MAdCAM-1, VCAM-1 or CS-1 of the respective integrin. In some embodiments the blocking agent binds to an integrin that has an α-4-subunit with an affinity constant of at least about 10⁻⁵ M. In some embodiments the affinity constant has a value of at least about 10⁻⁶ M. The binding affinity may in some embodiments be of a K_{D} of about 0.1 nM or below. In some embodiments the K_{D} may be below 10 picomolar (pM). An α₄-integrin blocking agent may in some embodiments bind to VLA-4 integrin. In some embodiments the α₄-integrin blocking agent binds to LPAM-1 integrin. In some embodiments the α₄-integrin blocking agent binds to both VLA-4 and LPAM-1 integrins. An α₄-integrin blocking agent may be an α₄-integrin binding antibody such as an anti-α₄-integrin immunoglobulin or a fragment of an anti-α₄-integrin immunoglobulin (cf. below for details). Examples of an α₄-integrin blocking agent include, but are not limited to, monoclonal immunoglobulins Natalizumab (Biogen, U.S. Pat. No. 5,840,299), Vedolizumab (Millennium Pharmaceuticals, Cambridge, U.S.), HP1/2, HP2/1, HP1/3 (Elices et al, Cell (1990) 60, 577-584), HP1/2 (Sanchez-Madrid et al, Eur. J. Immunol. (1986) 16, 1343-1349), humanized HP1/2 (U.S. Pat. No. 6,602,503), HP1/7, HP2/4, B-5G10, Max68P (Becton Dickinson GmBH, Germany), L25 (Becton Dickinson GmBH, Germany), P₄C₂ (Abeam, Cambridge, UK), R1-2 (BD Biosciences) and AJM300 (Ajinomoto, Japan).

The terms “comprising”, “including,” containing”, “having” etc. shall be read expansively or open-ended and without limitation. Singular forms such as “a”, “an” or “the” include plural references unless the context clearly indicates otherwise. Thus, for example, reference to a “vector” includes a single vector as well as a plurality of vectors, either the same—e.g. the same operon—or different. Likewise reference to “cell” includes a single cell as well as a plurality of cells. Unless otherwise indicated, the term “at least” preceding a series of elements is to be understood to refer to every element in the series. The terms “at least one” and “at least one of” include for example, one, two, three, four, or five or more elements. It is furthermore understood that slight variations above and below a stated range can be used to achieve substantially the same results as a value within the range. Also, unless indicated otherwise, the disclosure of ranges is intended as a continuous range including every value between the minimum and maximum values.

The scope and meaning of any use of a term will be apparent from the specific context in which the term is used. Certain further definitions for selected terms used throughout this document are given in the appropriate context of the detailed description, as applicable. Unless otherwise defined, all other scientific and technical terms used in the description, figures and claims have their ordinary meaning as commonly understood by one of ordinary skill in the art.

### Assessment of PML Risk and the Sample Used

The present invention is at least in part based on the surprising finding that CD62L levels and PSGL-1 levels on T cells can be used as an indicator for the risk evaluation of occurrence of PML in a subject, for example a subject having an organism that is in a condition associated with immunosuppression. On the basis of CD62L levels and/or PSGL-1 levels, optionally in conjunction with further indicators or tests explained in this specification, it can be assessed whether a subject is more likely to suffer from PML, for example when compared to a healthy subject or when compared to the statistical average of subjects that are in a comparable health state. The levels of PSGL-1 and CD62L on T cells can assist a physician in the determination of an appropriate therapeutic regimen. In some embodiments the subject is a subject infected with HIV and/or a subject undergoing HAART. A CD62L level on T cells can be used for identifying a likelihood that an HIV positive subject such as a subject suffering from AIDS will develop PML, as well as for predicting whether a subject undergoing HAART will develop PML. Determining levels of CD62L and/or of PSGL-1 on T cells improves the assessment of potential HIV complications and facilitates decision making with regard to the further course of HIV therapy and/or HAART.

In part the present invention is also based on the finding that the binding of VLA-4 influences the expression of the cell surface molecules CD62L, PSGL-1 and LFA-1 and basic immune cell functions such as migratory capacity. Without being bound by any, particular theory, the present inventors have discovered that some cell surface molecules, including CD62L, PSGL-1 and LFA-1, are differentially expressed on T cells in subjects who/that develop or have developed PML. In addition, the present inventors have found that CD62L is already differently expressed on T cells in subjects who/that are about to develop PML. Use of such molecules as biomarkers, optionally in conjunction with further biomarkers or tests, provides an indication as to which subjects are more likely to suffer from PML. The biomarkers provided in the present invention can assist physicians in determining an appropriate therapeutic regimen. Without being bound by any particular theory, the inventors' findings may help understand a previous observation by Wipfler et al. (Multiple Sclerosis (2011) 17, 1, 16-23), who reported a decrease in the expression of unblocked CD49d (α4-integrin) on mononuclear cells in blood of patients under treatment with the (α4β1 and (α4β7 integrin inhibiting immunoglobulin Natalizumab.

Accordingly, the biomarkers provided in the present invention can be advantageously used to diagnose the immune competence of a subject, such as a subject who/that is in a state of immunodeficiency, for instance a therapy to prevent graft rejection or a therapy with an α₄-integrin blocking agent. A respective subject may be immunocompromised due to an infection such as an infection with HIV. A respective subject may be immunocompromised due to receiving an immunosuppressive therapy, including therapy for graft-versus-host disease or therapy after having received an organ transplant. An immunosuppressive therapy may also be a therapy for an autoimmune disease such as multiple sclerosis, Crohn's disease, rheumatoid arthritis, systemic lupus erythematosus, diabetes mellitus type I or an idiopathic inflammatory myopathy such as dermatomyositis, polymyositis and sporadic inclusion body myositis. The biomarkers described in this document can also be used to diagnose the immune competence of a subject receiving or expected to receive long-term α₄-integrin blocking agent, VLA-4 blocking agent and/or a LPAM-1 blocking agent treatment or of a subject who/that is HIV positive. The biomarkers described in this document can also be used to diagnose the risk of the subject to suffer from PML. Diagnosing or detecting enhanced risk of PML occurrence can help in modifying a current therapy of a subject or initiate a therapy in order to reduce the risk of PML occurrence. In some embodiments the above biomarkers may be used in panels that include more than one biomarker, for risk stratification, for diagnosis of existing PML, for monitoring for a risk level, including for a potential risk increase, of PML, and for predicting a future medical outcome, such as improved or worsening immunosuppressive therapy and/or of HIV therapy, with regard to the occurrence of a JCV-induced disease in a subject. While HIV infection remains the most common predisposing factor for PML, PML can for instance also occur as a complication of a condition, in particular a chronic illness, associated with secondary immunosuppression such as Lupus Erythematosus (supra). As indicated in the introduction, PML has also been found to be associated with the use of the anti-CD20 monoclonal antibody Rituximab, used in the treatment of lymphomas. After the priority dates of the present application a case has also been reported where PML occurred after combination treatment with Rituximab and the alkylating agent Bendamustine, where an association with Bendamustine was suspected (Warsch, S, et al., Int J Hematol. (2012) 96, 2, 274-278). As a further example, PML has also been reported as a complication of polymyositis. It is understood that a method of the invention can be applied to any such condition, where applicable with an adjustment of the medication of a treatment to which the subject is being exposed.

PML is a formerly rare, but severe, subacute, rapidly progressive demyelinating disease of the brain, which was first characterized in 1958. PML has today reached epidemic proportions, mostly due to the fact that HIV/AIDS has resulted in a remarkable increase in the frequency of PML. In some locales, HIV infection has been found to account for more than 90% of the predisposing disorders associated with PML. As indicated above, PML is caused by a lytic infection of oligodendroglia cells with JCV in the brain. JCV infects children, and seropositivity in adults is reported to be between 50% and 60%, with higher prevalence in men than in women (Soelberg Sørensen, P., et al., Multiple Sclerosis Journal (2012) 18, 2, 143-152). It should be noted that seropositivity appears to increase with age. Likewise, seropositivity appears to increase with duration of Natalizumab exposure (Outteryck, O., et al., J Neurol (2012) DOI 10.1007/s00415-012-6487-5). JCV was first isolated in 1971 from brain tissue of a patient with Hodgkin's lymphoma who developed PML. The virus has a supercoiled double-stranded DNA genome. The mode of transmission of JCV has so far not been well defined, although respiratory transmission is suspected.

JCV infection of cells is initiated by attachment of the viral protein 1 (VP1) of JCV to the oligosaccharide lactoseries tetrasaccharide c (LSTc) on host cells (Neu, U., et al., Cell Host & Microbe (2010) 8, 309-319). The non-enveloped JCV virion is then taken up into cells via clathrin dependent receptor-mediated endocytosis. The supposedly transmittable form of JCV has commonly been referred to as the JCV archetype, as it has been assumed that all other genotypes originate from it. These assumptions, are, however, so far not supported by sound evidence, i.e. it is not established whether the transmittable form of JCV is indeed the archetypal form of the virus. It is further not known whether JCV superinfections can occur after initial childhood infection (White, M. K., & Khalili, K., J. Infect. Disease [2011] 203, 5, 578-586). PML is thought to be caused by reactivation of JCV, which can stay latent in a variety of tissues such as the kidneys, the tonsils, B lymphocytes and lymphoid organs as well as the central nervous system. Fragments of JCV DNA have even been found in oligodendrocytes and astrocytes in non-PML brain. The archetypal form of JCV seems to be exclusively found in the kidneys of non-PML individuals. Pathological JCV PML-type variants, which always have, relative to the JCV archetype, an altered regulatory region, form in the host via an unknown mechanism. Compared to the JCV archetype, pathological JCV PML-type variants have been found to contain in >80% of cases an amino acid substitution in the major capsid protein, VP1, typically in one of the outer loops. Further, deletions, duplications, and point mutations in the noncoding regulatory region and/or the coding region, have been reported.

JCV causes lytic infection and death of myelin producing oligodendrocytes in the white matter. It also infects astrocytes in a non-productive fashion; an abortive infection can lead to multinucleated giant astrocytes. PML typically results in focal neurologic deficits such as aphasia, hemiparesis and cortical blindness. It is currently diagnosed by analysing cerebrospinal fluid or a brain biopsy specimen for the presence of JCV DNA.

Both PML incidents during HIV/AIDS and the risk of PML-attributable death have been found to decrease under HAART as described by Khanna et al. (Clinical Infectious Diseases 48, 1459-1466). This document is incorporated herein by reference in its entirety for all purposes. In case of conflict, the present specification, including definitions, will control. In HIV-infected individuals, the supposedly most effective strategy for fighting PML is to optimize HAART to completely suppress HIV viral load and allow the best CD4⁺ T-cell immune recovery. Since antiretroviral therapy does not have any effect on JCV replication in vitro, it's in vivo effect is thought to be solely due to immune restoration (Hernandez et al., 2009, supra). In this regard higher levels of CD4⁺ cell counts have been associated with an improved survival in several clinical observations. However, in the context of occurrence of PML, JCV specific T cell responses rather than the overall CD4⁺ cell count appear to be the factor critical for PML survival (Khanna et al., 2009, supra). One route of cell entry of JCV has been identified to involve the serotonin 5-HT₂ₐ receptor, so that it can be assumed that HT₂ₐ antagonists may be suitable for gaining time before immune reconstitution is achieved (Focosi, D, et al., The Neuroscientist [2010] 16, 3, 308-323). HT₂ₐ antagonists can, however, not clear the virus from the host. Furthermore, a case has been reported where the quinine analog mefloquine, available under the trade name Lariam® from Roche for the prevention and therapy of P. falciparum, i.e. malaria, was used to treat an HIV patient under antiretroviral therapy, who had developed PML (abstract of Adachi, E., et al., Int J STD & AIDS (2012) 23, 8, 603). With continued antiretroviral therapy the patient's neurological status was reported to have improved substantially. Mefloquine has been shown to inhibit the JC virus infection in vitro (Brickelmaier, M, et al., Antimicrob Agents Chemother (2009) 53, 1840-1849). A case has also been reported where mefloquine could be used to treat PML in a patient with relapsing-remitting MS during and after plasma exchange (Schröder, A., et al., Archives of Neurology (2010) 67, 11, 1391-1394).

In the context of treatment with an α₄-integrin blocking agent and/or a VLA-4 blocking agent, such as Natalizumab treatment, known risk factors for development of PML include the duration of exposure to the α₄-integrin/VLA-4 blocking agent, prior immunosuppressive therapy and the presence of anti-JCV antibodies (Soelberg Sørensen et al., 2012, supra). The elevated risks associated with prior use of immunosuppressants, the duration of exposure to the α₄-integrin VLA-4 blocking agent and presence of anti-JCV antibodies appear to be independent of each other. The overall incidence of PML is reported to be about two in 1000 Natalizumab-treated patients (ibid.). The earlier PML associated with an α₄-integrin/VLA-4 blocking agent is diagnosed and treated the better is the clinical outcome.

One method of the invention is a method of diagnosing or aiding in the diagnosis of the risk of development of a condition associated with JCV in a subject. JCV associated conditions and symptoms of PML generally include defects of motor and/or cognitive performance. Symptoms/conditions that may occur are for instance weakness, hemiparesis, hemiplegia, i.e. partial paralysis, ataxia, altered mental status, visual field disturbances including loss of vision, impaired speech including aphasia, cognitive deterioration, as well as the so called Alien hand syndrome.

A related method of the invention is a method of diagnosing or aiding in the diagnosis of the risk of occurrence of PML in a subject. The subject is in some embodiments infected with HIV. This method of assessing the risk of occurrence of PML may also be taken as a method of diagnosing the likelihood that the subject will develop PML or of diagnosing the predisposition of the subject to develop PML. It is understood that a respective diagnosis/assessment involves a valuation which may subsequently turn out to be less than 100% precise for a given individual. Such assessment is in some embodiments to be taken as an indication of the balance of probabilities rather than as a solid predication.

A respective method according to the present invention generally involves analysis of a sample from the subject in vitro. Typically the sample is, essentially consists of, or includes body fluid from the subject. The sample may in some embodiments be a whole blood sample from the subject. In some embodiments the sample is a blood cell sample. Such a sample contains cells of the blood, however without the serum, which may for instance have been removed by centrifugation. The sample is in some embodiments a lymph sample, taken from the subject at a previous point of time, including taken immediately before use in a method according to the invention. In some embodiments the sample is a sample of cerebrospinal fluid. In some embodiments the method may include providing a sample from the subject. The sample may have been taken at any desired point in time before carrying out the method of the invention. Generally time interval between taking the sample and carrying out the method of the invention is selected to allow analysis of viable cells. It is within the skilled artisan's experience to determine a respective time interval during which T cells in a sample can be expected to remain viable. As a general orientation, the inventors have found that in the form of EDTA blood, i.e. after adding a final amount of about 1-2 mg/ml EDTA (typically potassium EDTA), cells remain viable and suitable for carrying out a method according to the invention during a time interval of up to 48 hours during which the sample is kept in fluid form at room temperature, i.e. about 18° C. Cells may for instance be kept at a temperature in the range from about 2° C. to about 37° C., such as from about 4° C. to about 37° C. or below. In some embodiments the sample is kept at about 32° C. or below. In some embodiments the sample is kept at a temperature of about 25° C. or below. As an illustrative example, a whole blood sample may be kept at about 25° C. or below. As a further example a cerebrospinal fluid sample may be kept at about 25° C. or below. As yet a further example a lymph sample may be kept at about 25° C. or below. In some embodiments the sample is kept at a temperature of about 22° C. or below, such as about 18° C. or below. In some embodiments the sample is kept at about 15° C. or below, such as below 10° C. In some embodiments the sample is kept at about 4° C. or at about 8° C. As an illustrative example, a whole blood sample may be kept at about 8° C. or below. As a further example a cerebrospinal fluid sample may be kept at about 8° C. or below. As further explained below, biomarker expression on T cells in the sample may be compared to expression in a reference sample. Such a reference sample may in some embodiments be or have been kept at comparable or the same conditions for about the same period of time as the sample from the patient. The reference sample may in some embodiments be stored for essentially the same period of time as the sample from the patient. In some embodiments the reference sample may be stored at at least essentially the same temperature as the sample from the patient. The reference sample may have been obtained in at least essentially the same way as the sample from the patient. The reference sample may have been processed in at least essentially the same way as the sample from the patient.

In some embodiments the sample has been taken on the same or on the previous day, such as about 48 hours or about 42 hours, before the method of the invention is being carried out. As an illustrative example, the sample may be a blood cell sample taken about 48 hours before use in a method of the invention. The sample may also be a whole blood sample taken about 48 hours earlier, i.e. before carrying out a method of the invention. The sample may furthermore be a cerebrospinal fluid sample taken about 48 hours earlier. In some embodiments the sample has been taken about 36 hours before carrying out a method of the invention. In some embodiments the sample has been taken about 30 hours before carrying out a method of the invention. In some embodiments the sample has been taken about 28 hours or about 24 hours before the method of the invention is being carried out. The sample may for instance be a lymph sample, taken about 24 hours earlier. In some embodiments the sample may be a whole blood sample taken from the subject about 24 hours before carrying out a method of the invention. In some embodiments the sample is a sample of cerebrospinal fluid taken about 24 hours before use in a method according to the invention. In some embodiments the sample has been taken about 18 hours earlier. In some embodiments the sample has been taken about 15 hours before the method of the invention is being carried out. The sample may also have been taken about 12 hours earlier. As an illustrative example, the sample may be a whole blood sample taken from the subject about 12 hours before carrying out a method of the invention. In some embodiments the sample is a lymph sample, taken about 12 hours before use in a method according to the invention. In some embodiments the sample is a sample of cerebrospinal fluid taken from the subject about 12 hours before carrying out a method of the invention. The sample may also be a blood cell sample taken about 12 hours earlier. In some embodiments the sample has been taken about 10 hours earlier. In some embodiments the sample has been taken about 8 hours, about 6 hours or less before the method of the invention is being carried out. In some embodiments the sample has been taken within a period of up to about 48 hours, i.e. 0 to about 48 hours. The sample may for instance have been taken within about 48 hours and have been stored at about 25° C. or below. In some embodiments the sample has been taken within a period of up to about 42 hours. As an example, the sample may be a whole blood sample taken from the subject within a period of up to about 42 hours before carrying out a method of the invention. In some embodiments the sample is a lymph sample, taken within a period of up to about 42 hours before employing a method according to the invention. The sample may in some embodiments been taken within a period of up to about to about 36 hours. As an illustrative example, the sample may have been taken within about 36 hours and have been stored at about 37° C. or below. In some embodiments the sample has been taken within a period of up to about 30 hours before performing a method of the invention. As an example, the sample may for instance be a lymph sample, taken within up to about 30 hours earlier. In some embodiments the sample may be a whole blood sample taken from the subject within up to about 30 hours before carrying out a method of the invention. In some embodiments the sample is a sample of cerebrospinal fluid taken within a period of up to about 30 hours before use in a method according to the invention. The sample has In some embodiments been taken within a period of up to about 28 hours, up to about 24 hours, to about 18 hours, to about 15 hours or 0 to about 12 hours before a method of the invention is being carried out. As indicated above, the subject, also addressed as a patient or an individual in this document, from which/whom the sample has been obtained is an animal, including a human.

In some embodiments the sample from the individual is a frozen sample. Generally the sample is frozen within one of the above detailed time intervals, e.g. 0 to about 48 or 0 to about 42 hours, and/or at the above exemplified time points, such as about 48 hours, about 36 hours or less, after the sample has been obtained from the individual. A frozen sample may be formed by freezing an obtained sample after adding a cryoprotective agent such as DMSO, glycerol and/or hydroxyethyl starch. In some embodiments, for instance where the sample is a blood cell sample, serum may in addition be added before freezing. As an illustrative example DMSO may be used in a final concentration in the range from about 2% to about 10%, such as about 2%, about 4%, about 5% or about 10% DMSO. Typically the sample is then frozen at a controlled rate to a temperature less than −50° C., whereafter the sample may for instance be stored, including long-term storage, at a temperature below −130° C. such as −160° C., e.g. in liquid nitrogen for extended periods of time.

In some embodiments of a method according to the invention a sample as provided from the individual is depleted of erythrocytes, in some embodiments at least essentially cleared of erythrocytes, if required. Depletion or removal of erythrocytes may for example be required in case the sample is a whole blood sample or a blood cell sample. Lysis of erythrocytes may be carried out osmotically or chemically. Osmotic lysis is suitable in the context of the present invention since erythrocytes lyse at an osmolarity at which leukocytes remain intact. In the art typically a solution of ammonium chloride is used for osmotic lysis, which may further include potassium bicarbonate and/or EDTA. A commercially available reagent may be used, such as the FCM Lysing solution by Santa Cruz (order no sc-3621), Erythrolyse Red Blood Cell Lysing Buffer by AbD Serotec or RBC Lysis Solution by 5 PRIME. Chemical lysis of erythrocytes may for example be achieved using an organic solvent such as diethylether or chloroform, and/or a surfactant, a copper containing solution or via adding one of certain bacterial or animal toxins. After lysis of erythrocytes the remaining blood cells may be collected, for example by means of centrifugation.

In a method according to the invention the level of T cells in the sample that have the protein(s) L-selectin, PSGL-1 and/or LFA-1 on their surface is detected. T cells are known to the skilled artisan as lymphocytes, i.e. nucleated blood cells that are also called white blood cells. T cells mature in the thymus and can be distinguished from other lymphocytes in that they have the T cell receptor on their cell surface. The main known role of the T cell is recognition of antigens bound to major histocompatibility complex (MHC) molecules. The T cell receptor (TCR) is a heterodimer, which in about 95% of T cells consists of a 34 kD α-chain, linked by a disulphide bond to a 34 kD β-chain. Both chains span the plasma membrane and have accordingly an extracellular portion, each of which includes a variable region, termed Vα and Vβ, respectively. About 5% of T cells have a T cell receptor that consists of a γ- and a δ-chain instead of an α- and a β-chain, which likewise have extracellular variable regions. T cell receptors can, like immunoglobulins, recognize a very large number of different epitopes.

In some embodiments the presence of the T cell receptor on the surface of a cell may be used to identify the cell as a T cell. As the T cell receptor has variable regions it may, nevertheless, be advantageous to use another cell surface protein to identify a T cell. An example of suitable protein in this regard is a T cell co-receptor. Two illustrative examples of a co-receptor of the T cell receptor are the protein complex CD3 (Cluster of Differentiation 3) and the protein CD247. CD3 has four chains, which are in mammals one D3γ chain, one CD3δ chain, and two CD3ε chains. These chains associate with a molecule known as the T-cell receptor and at least one T-cell surface glycoprotein CD3 zeta chain also known as T-cell receptor T3 zeta chain or CD247 (Cluster of Differentiation 247). CD247 may be present on the cell surface as either ζ₂ complex or a ζ/η complex. The complex of TCR, CD247 and CD can generate an activation signal in T lymphocytees. The TCR, ζ-chain(s), and CD3 molecule together define the TCR complex. In practicing a method according to the invention identifying the presence of CD3 on a particular cell or plurality of cells is often a convenient way of identifying T cells. Therefore the terms “CD3⁺ T cell” and “T cell” can generally be used interchangeable to address a T cell and to distinguish a T cell from other cell types.

A further example of a co-receptor of the T cell receptor, present on some but not all T cells, is the transmembrane protein CD8 (Cluster of Differentiation 8). Most T cells that have CD8 on their surface are cytotoxic T cells. CD8 plays an important role in binding to the class I major histocompatibility complex. Two isoforms of the protein, namely CD8-alpha and -beta, are known. Each such chain contains a domain that resembles an immunoglobulin variable domain. CD8 is a dimer of two of these chains, either a homo- or a heterodimer.

CD4⁺ T cells have, generally in addition to CD3, the CD4 (Cluster of Differentiation 4) protein on their surface, a glycoprotein consisting of four extracellular immunoglobulin domains, termed D₁ to D₄, and a small cytoplasmic region. The CD4 protein is known to be used by HIV-1 to gain entry into T cells of a host. CD4⁺ T cells can be classified into a variety of cell populations with different functions and should thus not be taken to define a unitary set of cells. Typical examples of a CD4⁺ T cell are a T helper cell, a regulatory T cell and a memory T cell.

In some embodiments a method according to the invention includes identifying CD3⁺ T cells in the sample, for example by employing an immunoglobulin, an immunoglobulin fragment or a proteinaceous binding molecule with immunoglobulin-like functions as further explained below. In some of these embodiments identifying CD3⁺ T cells in the sample serves in distinguishing CD3⁺ T cells from other cells such as CD3⁻ cells or non-T cells. In some embodiments CD4⁺ T cells are identified in the sample. Identifying CD4⁺ T cells typically serves in distinguishing CD4⁺ T cells from other cells such as CD4⁺ T cells or non-T cells. In some embodiments CD8⁺ T cells are identified in the sample. Identifying CD8⁺ T cells typically serves in distinguishing CD8⁺ T cells from other cells such as CD8⁻ T cells or non-T cells. It is understood that CD4⁺ T cells and CD8⁺ T cells are typically also CD3⁺ T cells so that a CD3⁺ T cell identified may also for instance be a CD4⁺ T cell rather than be distinguished from a CD4⁺ T cell. Accordingly, in some embodiments in a first step a T cell may be identified as a CD3⁺ T cell. In a second step it may be determined whether the CD3⁺ T cell is a CD4⁺ T cell. It may also be determined whether the CD3⁺ T cell is a CD8⁺ T cell. As explained above, in some embodiments T cells are identified by the presence of CD3. Of the thus identified T cells CD4⁺ T cells are distinguished from CD8⁺ T cells.

In some embodiments a method according to the invention includes enriching and/or isolating CD3⁺ T cells from the sample. In some embodiments a method according to the invention includes enriching and/or isolating CD4⁺ T cells and/or CD8⁺ T cells from the sample. In some embodiments T cells are enriched, including sorted, based on the presence of CD3 on the cell surface. Of the thus enriched T cells, those T cells that have CD4 on their surface, i.e. CD4⁺ T cells, may be further enriched. In some embodiments, of the T cells that have been enriched based on the presence of CD3, those T cells that have CD8 on their surface, i.e. CD8⁺ T cells, may be further enriched. As an illustrative example, the sample may be from an HIV positive individual. CD3⁺ T cells may be enriched in a first step, of which CD4⁺ T cells may be enriched in a second step, thereby obtaining enriched CD3⁺ CD4⁺ T cells. The CD3⁺CD4⁺ T cells of the HIV positive individual may then be used in a method according to the invention. Furthermore, CD8⁺ T cells may be enriched in a second step, thereby obtaining enriched CD3⁺CD8⁺ T cells from the HIV positive individual. CD8 positive T cells or CD4 positive T cells of the HIV positive individual may for instance be used to analyse the expression of PSGL-1 thereon. Likewise, CD4 positive T cells of the HIV positive individual may for example be used to analyse the expression of CD62L thereon. As a further illustrative example, where the HIV positive individual is of a stadium before stadium C3 (e.g. at stadium A1, A2, A3, B1, B2, B3, C1 or C2), CD8 positive T cells of the HIV positive individual may for example be used to analyse the expression of CD62L thereon. As a further illustrative example, the sample may be from an individual undergoing treatment with an α₄-integrin blocking agent, a VLA-4 blocking agent and/or a LPAM-1 blocking agent. CD3⁺ T cells may be enriched in a first step, of which CD4⁺ T cells may be enriched in a second step, thereby obtaining enriched CD3⁺CD4⁺ T cells. Likewise, CD8⁺ T cells may be enriched in a second step, thereby obtaining enriched CD3⁺CD8⁺ T cells. Both the CD3⁺CD4⁺ T cells and the CD3⁺CD8⁺ T cells of the HIV positive individual may then be used in a method according to the invention.

In some embodiments enriching and/or isolating CD3⁺ T cells, CD4⁺ T cells and/or CD8⁺ T cells from the sample includes cell sorting and/or selection, for instance via negative magnetic immunoadherence or flow cytometry. In some embodiments enriching and/or isolating such cells consist of cell sorting or selection. Such a technique may be based on contacting the cells with a plurality of antibodies directed to cell surface markers present on the cells negatively selected. As an illustrative example, to enrich for CD4⁺ cells by negative selection, a plurality of antibodies may include antibodies directed to CD14, CD20, CD11b, CD16, HLA-DR, and CD8, while to enrich for CD8⁺ cells by negative selection, a plurality of antibodies may include antibodies directed to CD14, CD20, CD11b, CD16, and HLA-DR.

In some embodiments it may be desired to enrich for or positively select for T cells that express CD3⁺. In some embodiments undesired cells are depleted by contacting them with particles/beads on which binding partners such as antibodies are immobilized that bind to proteins found on undesired cells, but not on desired cells. In some embodiments desired cells are collected from the sample by contacting them with beads on which binding partners such as antibodies are immobilized that bind to proteins found on the desired cells, but not on undesired cells.

For isolation of a desired population of cells by positive or negative selection, the amount and concentration of cells and particle/bead surface can be varied. In certain embodiments it may be desired to reduce the volume in which beads and cells are contacted, for instance to ensure maximum contact of cells and beads. For example, in one embodiment, a concentration of about 2 billion cells/ml is used. In one embodiment, a concentration of about 1 billion cells/ml is used. In a further embodiment, a concentration of more than about 100 million cells/ml is used. In some embodiments a concentration of cells of about 10 million cells/ml or more is used. In some embodiments cells are at a concentration of about 15, including about 20, about 25 or about 30 million cells/ml. In some embodiments a concentration of cells of about 35, about 40, about 45, about 50 million cells/ml or more is used. In some embodiments a concentration of cells of about 75 million cells/ml is used. In some embodiments cells are at a concentration of about 80 million cells/ml. In some embodiments cells are at a concentration of about 85 million cells/ml. The concentration of cells may for example be about 90, including about 95, about 100, or about 125 million cells/ml or more. In some embodiments a concentration of cells of about 150 million cells/ml or more is used. The use of high cell concentrations may in some embodiments result in increased cell yield, cell activation, and cell expansion. In some embodiments the use of high cell concentrations may allow more efficient capture of cells that may express e.g. CD62L or PSGL-1 in low number.

Where desired, further matter may be added to the sample for analysis, for example dissolved or suspended in the sample. It is understood that any dilution due to such addition of matter has to be accounted for and may need to be considered when calculating the level of L-selectin (CD62L) expressing T cells. Likewise, any dilution due to the addition of matter has to be accounted for and may need to be considered when calculating the level of PSGL-1 expressing T cells. As an illustrative example one or more buffer compounds may be added to the sample. Numerous buffer compounds are used in the art and may be used to carry out the various methods described herein. Examples of buffers include, but are not limited to, solutions of salts of phosphate, carbonate, succinate, carbonate, citrate, acetate, formate, barbiturate, oxalate, lactate, phthalate, maleate, cacodylate, borate, N-(2-acetamido)-2-amino-ethanesulfonate (also called (ACES), N-(2-hydroxy-ethyl)-piperazine-N′-2-ethanesulfonic acid (also called HEPES), 4-(2-hydroxyethyl)-1-piperazine-propanesulfonic acid (also called HEPPS), piperazine-1,4-bis(2-ethanesulfonic acid) (also called PIPES), (2-[tris(hydroxymethyl)-methylamino]-1-ethansulfonic acid (also called TES), 2-cyclo-hexyl-amino-ethansulfonic acid (also called CHES) and N-(2-acetamido)-iminodiacetate (also called ADA). Any counter ion may be used in these salts; ammonium, sodium, and potassium may serve as illustrative examples. Further examples of buffers include, but are not limited to, triethanolamine, diethanolamine, ethylamine, triethylamine, glycine, glycylglycine, histidine, tris(hydroxymethyl)-aminomethane (also called TRIS), bis-(2-hydroxyethyl)-imino-tris(hydroxymethyl)methane (also called BIS-TRIS), and N-[Tris(hydroxymethyl)-methyl]glycine (also called TRICINE), to name a few. A respective buffer may be an aqueous solution of such buffer compound or a solution in a suitable polar organic solvent. Further examples of matter that may be added to the sample include salts, detergents or chelating compounds. As yet a further illustrative example, nuclease inhibitors may need to be added in order to maintain a nucleic acid molecule in an intact state.

### Biomarkers

In some embodiments of a method according to the invention the level of L-selectin (CD62L) expressing T cells, such as CD3⁺ T cells, including CD4⁺ T cells and/or CD8⁺ T cells, in the sample is detected. In some embodiments of a method according to the invention the level of PSGL-1 expressing T cells, such as CD3⁺ T cells, in the sample is detected. In some embodiments the level of both CD62L and PSGL-1 expressing T cells, e.g. CD62L and PSGL-1 expressing CD3⁺ T cells, in the sample is detected.

The protein L-selectin may be any respective variant or isoform of the respective species, e.g. human. The protein may for example be the human protein of the Swissprot/Uniprot accession number P14151 (version 145 as of 22 Feb. 2012) or the human protein of the Swissprot/Uniprot accession number Q9UJ43 (version 97 as of 22 Feb. 2012). This protein may for instance be encoded by the SELL gene of GenBank accession number NG_{—}016132 (version NG_{—}016132.1 as of 1 Feb. 2012; GI:270047500). The protein may for example be encoded by the mRNA of GenBank accession number BC020758 (version BC020758.1 as of 4 Aug. 2008; GI: 18088807). The protein may in some embodiments be the mouse protein of the Swissprot/Uniprot accession number P18337 (version 121 as of 11 Jul. 2012), the mouse protein of the Swissprot/Uniprot accession number B1B506 (version 39 as of 3 Oct. 2012), or the mouse protein of the Swissprot/Uniprot accession number Q3TCF3 (version 53 as of 3 Oct. 2012). In some embodiments the protein may be the rat protein of the Swissprot/Uniprot accession number P30836 (version 94 as of 22 Feb. 2012) or the rat protein of the Swissprot/Uniprot accession number Q63762 (version 89 as of 22 Feb. 2012). The protein may also be the bovine protein of the Swissprot/Uniprot accession number P98131 (version 82 as of 22 Feb. 2012) or the bovine protein of the Swissprot/Uniprot accession number F1N4U9 (version 13 as of 3 Oct. 2012). In some embodiments the protein may be the horse protein of the Swissprot/Uniprot accession number F7E0Z9 (version 12 as of 3 Oct. 2012). The protein may also be the rhesus macaque protein of the Swissprot/Uniprot accession number F6VQ43 (version 11 as of 3 Oct. 2012), the rhesus macaque protein of the Swissprot/Uniprot accession number Q95198 (version 85 as of 3 Oct. 2012) or the rhesus macaque protein of the Swissprot/Uniprot accession number H9YUD6 (version 3 as of 3 Oct. 2012). The protein may also be the chimpanzee protein of the Swissprot/Uniprot accession number Q95237 (version 87 as of 3 Oct. 2012). In some embodiments the protein may be the protein of the crab-eating macaque (Macaca fascicularis) with Swissprot/Uniprot accession number G8F369 (version 5 as of 3 Oct. 2012). In some embodiments the protein may be the protein of the Sumatran orangutan with Swissprot/Uniprot accession number H2N4S6 (version 7 as of 3 Oct. 2012) or the protein of the Sumatran orangutan with Swissprot/Uniprot accession number H2N4S5 (version 7 as of 3 Oct. 2012). In some embodiments the protein may be the protein of the Bornean orangutan with Swissprot/Uniprot accession number Q95235 (version 78 as of 3 Oct. 2012). The protein may also be the protein of the Northern white-cheeked gibbon (Nomascus leucogenys) with the Swissprot/Uniprot accession number G1RYC8 (version 10 as of 3 Oct. 2012).

L-selectin mediates lymphocyte homing to high endothelial venules of peripheral lymphoid tissue and leukocyte rolling on activated endothelium at inflammatory sites. Most peripheral blood B cells, T cells, monocytes and granulocytes express CD62L/L-selectin. However, some natural killer cells, spleen lymphocytes, bone marrow lymphocytes, bone marrow myeloid cells, thymocytes, and certain hematopoietic malignant cells also express CD62L. Its expression is commonly used to differentiate between central- and effector-memory T cells.

In an embodiment of a method according to the invention the level of LFA-1 expressing T cells in the sample is detected. LFA-1 is an integrin-type cell adhesion molecule that is predominantly involved in leukocyte trafficking and extravasation. LFA-1 binds to CD54, the Intercellular Adhesion Molecule 1, on antigen-presenting cells. LFA-1 is a heterodimer having a (3-chain, termed CD18, and an [symbol] -chain, termed CD11a. Both the [symbol] -chain and the (3-chain contain a von Willebrand factor type A domain (VWFA domain) in their N-terminal portion, also called inserted domain (1-domain) that plays a central role in regulating ligand binding. Also known as CD11a/CD18 or integrin [symbol] _{L} [symbol] ₂, LFA-1 plays a crucial role in many cellular and immunological processes (migration, antigen presentation, cytotoxicity, cell proliferation and haematopoiesis) by displaying both signaling and adhesive properties. LFA-1 is the primary integrin receptor involved in leukocyte arrest on inflamed endothelium.

The protein CD18 (integrin beta-2) may be any respective variant or isoform of the respective species, e.g. human. In some embodiments CD18 is the human protein of the Swissprot/Uniprot accession number Swissprot/Uniprot accession number P05107 (version 169 as of 11 Jul. 2012), the human protein of the Swissprot/Uniprot accession number Q6PJ75 (version 60 as of 11 Jul. 2012) or the human protein of the Swissprot/Uniprot accession number B4E021 (version 26 as of 16 May 2012). The protein may also be the goat protein of the Swissprot/Uniprot accession number Q5VI41 (version 44 as of 11 Jul. 2012), the porcine protein of the Swissprot/Uniprot accession number P53714 (version 88 as of 11 Jul. 2012), the bovine protein of the Swissprot/Uniprot accession number P32592 (version 105 as of 11 Jul. 2012), the chimpanzee protein of the Swissprot/Uniprot accession number Q5NKT5 (version 56 as of 11 Jul. 2012) or the rhesus macaque protein of the Swissprot/Uniprot accession number H9Z8N5 (version 2 as of 11 Jul. 2012). CD18 may be the protein encoded by the ITGB2 gene, for example the mouse gene of GenBank Gene ID No 12575 as of 19 Feb. 2012 or the human gene of GenBank Gene ID No 3689 as of 19 Feb. 2012.

The protein CD45 may be any respective variant or isoform of the respective species, e.g. human. The protein may for example be the human protein of the Swissprot/Uniprot accession number P20701 (version 137 as of 22 Feb. 2012) or the human protein of the Swissprot/Uniprot accession number Q96HB1 (version 76 as of 22 Feb. 2012). The protein may also be the mouse protein of the Swissprot/Uniprot accession number P24063 (version 108 as of 22 Feb. 2012) or the bovine protein of the Swissprot/Uniprot accession number P61625 (version 56 as of 22 Feb. 2012). CD45 may be the protein encoded by the ITGAL gene, such as the human gene of GenBank Gene ID No 3683 as of 5 Feb. 2012, the bovine gene of GenBank Gene ID No 281874 as of 4 Feb. 2012 or the mouse gene of GenBank Gene ID No 16408 as of 14 Feb. 2012.

In one embodiment of a method according to the invention the level of PSGL-1 expressing T cells in the sample is detected. P-selectin glycoprotein ligand-1 (PSGL-1), also termed ELPLG, CLA, Selectin P ligand or CD162 (cluster of differentiation 162), is a 240 kDa homodimer consisting of two 120 kD polypeptide chains. PSGL-1 is a heavily glycosylated sialomucin constitutively expressed on most leukocytes. PSGL-1 can bind to the three selectins, P-, E- and L-selectin, and is an adhesion receptor mediating inter alia leukocyte tethering and activation of stable adhesion. PSGL-1 on circulating monocytes can for instance interact with P- or E-selectin to tether monocytes to endothelium. PSGL-1 appears to be the major molecule mediating leukocyte-endothelium interactions and leukocyte rolling on stimulated endothelium. The protein has been found to be critical in transition from slow rolling to arrest and for efficient transendothelial migration. PSGL-1 is also a facilitator of resting T cell homing into lymphoid organs. Further, PSGL-1 has been reported to transduce an intracellular signal that converts LFA-1 into a partially activated state, in which LFA-1 is able to interact with ICAM-1 (Lefort, C. T, and Ley, K., Frontiers in Immunology (2012), 3, article 157).

The protein PSGL-1 may be any respective variant or isoform of the respective species, e.g. human. The protein may for example be the human protein of the Swissprot/Uniprot accession number Q14242 (version 110 as of 11 Jul. 2012), the human protein of the Swissprot/Uniprot accession number B4DHR9 (version 15 as of 13 Jun. 2012) or the human protein of the Swissprot/Uniprot accession number B7Z5C7 (version 21 as of 13 Jun. 2012). PSGL-1 may also be the mouse protein of the Swissprot/Uniprot accession number Q62170 (version 87 as of 11 Jul. 2012), the mouse protein of the Swissprot/Uniprot accession number Q99L34 (version 49 as of 13 Jun. 2012), the mouse protein of the Swissprot/Uniprot accession number Q3TA56 (version 49 as of 11 Jul. 2012), the dog protein of the Swissprot/Uniprot accession number F7J212 (version 6 as of 13 Jun. 2012), the rat protein of the Swissprot/Uniprot accession number Q8K5B0 (version 45 as of 22 Feb. 2012), the naked mole rat protein of the Swissprot/Uniprot accession number G5AWZ5 (version 3 as of 22 Feb. 2012) or the hamster protein of the Swissprot/Uniprot accession number G3HI97 (version 2 as of 25 Jan. 2012). In some embodiments the protein may be the bovine protein of the Swissprot/Uniprot accession number F1MS77 (version 7 as of 11 Jul. 2012), the gorilla protein of the Swissprot/Uniprot accession number G3R6X5 (version 5 as of 11 Jul. 2012), the gibbon protein of the Swissprot/Uniprot accession number G1R504 (version 5 as of 16 May 2012), the protein of the small-eared galago (Otolemur garnettii) of the Swissprot/Uniprot accession number H0Y0C0 (version 3 as of 16 May 2012), or the protein of the thirteen-lined ground squirrel (Spermophilus tridecemlineatus) of the Swissprot/Uniprot accession number 13N665 (version 1 as of 11 Jul. 2012). PSGL-1 may also be the potential PSGL-1 protein of the Sumatran orang-utan with the Swissprot/Uniprot accession number H2NIJ3 (version 2 as of 16 May 2012), the potential PSGL-1 protein of the chimpanzee with the Swissprot/Uniprot accession number H2RCX5 (version 2 as of 16 May 2012). The protein may also be the rhesus macaque protein isoform 1 of the Swissprot/Uniprot accession number H9EY51 (version 2 as of 3 Oct. 2012), the rhesus macaque protein isoform 2 of the Swissprot/Uniprot accession number H9EY58 (version 2 as of 3 Oct. 2012) or the rhesus macaque protein isoform 2 of the Swissprot/Uniprot accession number H9F2P7 (version 2 as of 3 Oct. 2012). The protein may also be the potential PSGL-1 protein of the crab-eating macaque (Macaca fascicularis) with the Swissprot/Uniprot accession number G7PI56 (version 1 as of 25 Jan. 2012) or the potential PSGL-1 protein of Guinea pig with the Swissprot/Uniprot accession number H0UVZ8 (version 4 as of 11 Jul. 2012). This protein may for instance be encoded by the human SELPLG gene of GenBank Gene ID No 6404 as of 25 Feb. 2012, the mouse SELPLG gene of GenBank Gene ID No 20345 as of 25 Feb. 2012 or the rat SELPLG gene of GenBank Gene ID No 363930 as of 11 Nov. 2011.

As explained above, a method according to the invention includes determining the amount or number of CD62L expressing, PSGL-1 expressing and/or LFA-1 expressing T cells, e.g. CD3 positive T cells. The level of expression, i.e. the amount present, of a protein, is determined by the rate of synthesis and the rate of degradation of the protein. The rate of synthesis of CD62L may for example be assessed by determining the synthesis rate of messenger RNA (mRNA) encoded by the selectin L (SELL) gene. Assessing de novo synthesis of a given protein alone, does, however, not result in information on the actual amount of the protein present in or on a cell, or in an organism. With knowledge of protein levels and de novo synthesis rate of a reference sample, such as a sample from a healthy subject, the skilled artisan can nevertheless generally perform a prediction in terms of relative protein levels. Synthesis of CD62L mRNA refers to any mRNA transcribed from a SELL gene (e.g. GenBank accession No. NG_{—}016132, version NG_{—}016132.1, GI: 70047500). Currently two transcript variants of human SELL are known, termed variant 1 (GenBank accession No. NM_{—}000655, version NM_{—}000655.4, GI:262206314) and variant 2 (GenBank accession No. NR_{—}029467, version NR_{—}029467.1; GI:262205323). Synthesis of CD18 mRNA refers to any mRNA transcribed from an ITGB2 gene. Synthesis of CD45 mRNA refers to any mRNA transcribed from an ITGAL gene.

Likewise, the rate of synthesis of PSGL-1 may in some embodiments be assessed by determining the synthesis rate of mRNA encoded by the selectin P ligand gene (SELPLG). Synthesis of SELPLG mRNA refers to any mRNA transcribed from a SELPLG gene, such as human mRNA. Currently two transcript variants of human SELPLG are known, termed variant termed variant 1 (GenBank accession No. NM_{—}001206609, version NM_{—}001206609.1, GI:331284237) and variant 2 (GenBank accession No. NM_{—}003006, version NM_{—}003006.4; GI:331284235). Further examples of PSGL-1 mRNA, the synthesis of which may be determined, include, but are not limited to, mouse mRNA with the sequence of GenBank accession No. NM_{—}009151 (version NM_{—}009151.3, GI:159110802), bovine mRNA with the sequence of GenBank accession No. NM_{—}001037628 (version NM_{—}001037628.1, GI:83035126), porcine mRNA with the sequence of GenBank accession No. NM_{—}001105307 (version NM_{—}001105307.1, GI:157427735), dog mRNA with the sequence of GenBank accession No. NM_{—}001242719 (version NM_{—}001242719.1, GI:337298526), horse mRNA with the sequence of GenBank accession No. NM_{—}001105161 (version NM_{—}001105161.1, GI:157364981) or chimpanzee mRNA with the sequence of GenBank accession No. XM_{—}001164136 (version XM_{—}001164136.2, GI:332840289.

The rate of synthesis of LFA-1 may in some embodiments be detected by determining the synthesis rate of mRNA encoded by the ITGAL gene and the ITGB2 gene. Synthesis of ITGAL mRNA refers to any mRNA transcribed from an ITGAL gene. Currently two transcript variants of the human integrin alpha L gene are known, termed variant 1 (GenBank accession No. NM_{—}002209, version NM_{—}002209.2, GI:167466214) and variant 2 (GenBank accession No. NM_{—}001114380, version NM_{—}001114380.1; GI:167466216). Human mRNA of the human ITGAL gene may also have or include the sequence of GenBank accession No. BC008777 (version BC008777.2, GI:33870544). Four transcript variants of the mouse ITGAL gene are known, termed variant 1 (GenBank accession No. NM_{—}001253872, version NM_{—}001253872.1, GI:359751454), variant 2 (GenBank accession No. NM_{—}008400, version NM_{—}008400.3; GI:359751456), variant 3 (GenBank accession No. NM_{—}001253873, version NM_{—}001253873.1; GI:359751457) and variant 4 (GenBank accession No. NM_{—}001253874, version NM_{—}001253874.1; GI:359751459). Further illustrative examples of ITGAL mRNA the synthesis rate of which may be analysed, are dog mRNA with the sequence of GenBank accession No. XM_{—}547024 (version XM_{—}547024.2, GI:73958404), wild boar mRNA with the sequence of GenBank accession No. EF585976 (version EF585976.1, GI:156601155) and rat mRNA with the sequence of GenBank accession No. BC101849 (version BC101849.1, GI:74353690).

Synthesis of ITGB2 mRNA refers to any mRNA transcribed from an ITGB2 gene. Currently two transcript variants of the human integrin beta 2 gene are known, termed variant 1 (GenBank accession No. NM_{—}000211, version NM_{—}000211.3, GI:188595673) and variant 2 (GenBank accession No. NM_{—}001127491, version NM_{—}001127491.1; GI:188595676). Human mRNA of the human ITGAL gene may also have or include the sequence of GenBank accession No. S75297 (version S75297.1; GI:242219). Further examples of ITGB2 mRNA, the synthesis of which may be determined, include, but are not limited to, mouse mRNA with the sequence of GenBank accession No. NM_{—}008404 (version NM_{—}008404.4, GI: 14.5966904), rat mRNA with the sequence of GenBank accession No. NM_{—}001037780 (version NM_{—}001037780.2, GI:163937848), dog mRNA with the sequence of GenBank accession No. XM_{—}849290 (version XM_{—}849290.3, GI:359323519) and chicken mRNA with the sequence of GenBank accession No. NM_{—}205251 (version NM_{—}205251.1, GI:46048727).

### Determining the Level of a Biomarker

In the context of the present invention the terms “detect” or “detecting” typically refer to a method that can be used to determine the amount of a nucleic acid or a protein, or an assessment from which such an amount can be inferred. Examples of such methods include, but are not limited to, RT-PCR, RNAse protection assay, Northern analysis, Western analysis, ELISA, radioimmunoassay or fluorescence titration assay. Assessing the amount of a biomarker such as PSGL-1 or CD62L in/on a cell may include assessing the amount of a nucleic acid, e.g. RNA, in a cell encoding the respective biomarker. A nucleic acid probe may be used to probe a sample by any common hybridization method to detect the amount of nucleic acid molecules of the e.g. PSGL-1 or CD62L protein. In order to obtain nucleic acid probes chemical synthesis can be carried out. The synthesized nucleic acid probes may be first used as primers in a polymerase chain reaction (PCR) carried out in accordance with recognized PCR techniques, essentially according to standard PCR protocols utilizing the appropriate template, in order to obtain the probes of the present invention. One skilled in the art will readily be able to design such probes based on the sequence available for the biomarker. The hybridization probes can be labeled by standard labeling techniques such as with a radiolabel, enzyme label, fluorescent label, biotin-avidin label, chemiluminescence or a nanoparticle. After hybridization, the probes may be visualized using a standard technique. As explained above, the rate of synthesis of a protein does not equal the expression of the protein, since the degradation rate of the protein likewise contributes to the expression level. Nevertheless, a change or a deviation in the rate of synthesis can generally be taken as an indication on a change or a deviation in the expression level of a protein.

The rate of synthesis of CD62L, PSGL-1, CD18 and/or CD45 may also be assessed by determining the synthesis rate of the respective protein/polypeptide, including the post-translational modifications of the initial translation product. CD62L is for example synthesized in the form of a pro-L-selectin after removal of the N-terminal signal peptide, which directs the protein to its cell membrane location. L-selectin is then formed after removal of the N-terminal propeptide. Further, a plurality of N-linked glycosylations occur. Likewise, CD162 is for example synthesized in the form of a pro-protein after removal of the N-terminal signal peptide. Removal of the N-terminal propeptide yields the mature protein PSGL-1. CD162 has complex, core-2, sialylated and fucosylated O-linked oligosaccharides and contains the Sialyl-Lewis^{x} (sLe^{x}) glycan. Further, CD162 is postranslationally modified by sulfation, which is required for P- and L-selectin binding. Any of these synthesis steps may be detected alone or in combination, for example based on the accumulation of products of a post-translational modification. It should be noted that resting and activated T cells have different glycosylation profiles and have for example different glycoforms of PSGL-1 on the cell surface.

Any method that can be used to detect the presence of a nucleic acid or a protein in the context of the present invention. Such a method may include established standard procedures well known in the art. Examples of such techniques include, but are not limited to, RT-PCR, RNAse protection assay, Northern analysis, Western analysis, ELISA, radioimmunoassay or fluorescence titration assay. Assessing the amount of a biomarker such as PSGL-1 or CD62L in/on a cell may include assessing the amount of a nucleic acid, e.g. RNA, in a cell encoding the respective biomarker. A nucleic acid probe may be used to probe a sample by any common hybridization method to detect the amount of nucleic acid molecules of the biomarker. In order to obtain nucleic acid probes chemical synthesis can be carried out. The synthesized nucleic acid probes may be first used as primers in a polymerase chain reaction (PCR) carried out in accordance with recognized PCR techniques, essentially according to standard PCR protocols utilizing the appropriate template, in order to obtain the respective probe. One skilled in the art will readily be able to design such a probe based on the sequences available for the biomarker. The hybridization probe can be labeled by standard labeling techniques such as with a radiolabel, enzyme label, fluorescent label, biotin-avidin label, chemiluminescence or a nanoparticle. After hybridization, the probes may be visualized using a standard technique.

A detection method used in the context of the present invention may include an amplification of the signal caused by the nucleic acid or protein, such as a polymerase chain reaction (PCR) or the use of the biotin-streptavidin system, for example in form of a conjugation to an immunoglobulin, as also explained in more detail below. The detection method may for example include the use of an antibody, e.g. an immunoglobulin, which may be linked to an attached label, such as for instance in Western analysis or ELISA. Where desired, an intracellular immunoglobulin may be used for detection. Some or all of the steps of detection may be part of an automated detection system. Illustrative examples of such systems are automated real-time PCR platforms, automated nucleic acid isolation platforms, PCR product analysers and real-time detection systems. As indicated above, the term “antibody” as used herein, is understood to include an immunoglobulin and an immunoglobulin fragment that is capable of specifically binding a selected protein, e.g. L-selectin or a protein specific for T cells, as well as a respective proteinaceous binding molecule with immunoglobulin-like functions. An antibody may for instance be an EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, an LDL-receptor class A domain, a Sushi domain, a Link domain, a Thrombospondin type I domain, an immunoglobulin domain or a an immunoglobulin-like domain (for example a domain antibody or a camel heavy chain antibody), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, a “Kappabody” (Ill. et al., Protein Eng (1997) 10, 949-957), a “Minibody” (Martin et al., EMBO J (1994) 13, 5303-5309), a “Diabody” (Holliger or al., PNAS U.S.A. 90, 6444-6448 (1993)), a “Janusin” (Traunecker et al., EMBO J. (1991) 10, 3655-3659 or Traunecker et al., Int J Cancer (1992) Suppl 7, 51-52), a nanobody, an adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein (cf. also below).

A measurement of a level or amount may for instance rely on spectroscopic, photochemical, photometric, fluorometric, radiological, enzymatic or thermodynamic means. An example of a spectroscopical detection method is fluorescence correlation spectroscopy. A photochemical method is for instance photochemical cross-linking. The use of photoactive, fluorescent, radioactive or enzymatic labels respectively are examples for photometric, fluorometric, radiological and enzymatic detection methods. An example of a thermodynamic detection method is isothermal titration calorimetry. As an illustrative example of a label, a detailed protocol on the use of water-soluble, bio-functionalized semiconductor quantum dots has been given by Lidke et al. (Current Protocols in Cell Biology, [2007] Suppl. 36, 25.1.1-25.1.18). Such quantum dots have a particularly high photostability, allowing monitoring their localization for minutes to hours to days. They are typically fluorescent nanoparticles. Since different types of quantum dots can be excited by a single laser line multi-colour labelling can be performed. Detection can for example conveniently be carried out in different fluorescence channels of a flow cytometer. A quantum dot can be coupled to a binding partner of PSGL-1, CD62L or LFA-1 as well as to a capture molecule (cf. below).

The measurement used is generally selected to be of a sensitivity that allows detection of CD62L, PSGL-1 and/or LFA-1 expressing cells in the range of a selected threshold value, in particular of a sensitivity that allows determining whether CD62L, PSGL-1 and/or LFA-1 expressing cells are below the threshold value. Typically a binding partner of CD62L, PSGL-1 and LFA-1, respectively, may be used in combination with a detectable marker. Such a binding partner of CD62L, PSGL-1 and/or LFA-1 has a detectable affinity and specificity for CD62L, PSGL-1 and LFA-1, respectively. Typically, binding is considered specific when the binding affinity is higher than 10⁻⁶ M. A binding partner of CD62L, PSGL-1 and LFA-1, respectively, has in some embodiments an affinity of about 10⁻⁸ M or higher, or of about 10⁻⁹ M or higher. As indicated above, in some embodiments T cells in the sample are identified by the presence of the CD3 protein on their surface; or T cells may be enriched or isolated via the presence of the CD3 protein on their surface. Identification of CD3⁺ T cells may again be carried out using spectroscopic, photochemical, photometric, fluorometric, radiological, enzymatic or thermodynamic means. Identification and enrichment or isolation of T cells may likewise be carried out by using a suitable binding partner of CD3⁺. Accordingly the above said applies mutatis mutandis to identifying and enriching or isolating T cells. Further, T cells may be identified or isolated in a similar manner, using suitable surface proteins known in the art, for example the T cell receptor. In some embodiments a suitable binding partner of CD3 and a further suitable binding partner of a surface protein characteristic for T cells such as the T cell receptor are combined to identify CD3⁺ T cells. Typically a binding partner of CD3 may be used in combination with a detectable marker. Likewise a binding partner of CD3 may be used in combination with a detectable marker. In some embodiments a suitable binding partner of CD3, a suitable binding partner of a surface protein characteristic for T cells such as the T cell receptor and a suitable binding partner of CD62L are combined to identify CD62L expressing CD3 T cells. In some embodiments a suitable binding partner of CD3⁺, a suitable binding partner of a surface protein characteristic for T cells such as the T cell receptor and a suitable binding partner of LFA-1 are combined to identify LFA-1 expressing CD3⁺ T cells. In some embodiments a suitable binding partner of CD3 and a suitable binding partner of CD62L are combined to identify CD62L expressing T cells. In some embodiments a suitable binding partner of CD3 and a suitable binding partner of PSGL-1 are combined to identify PSGL-1 expressing T cells. In some embodiments a suitable binding partner of CD3⁺ and a suitable binding partner of LFA-1 are combined to identify LFA-1 expressing T cells. In some embodiments a suitable binding partner of CD3⁺, a suitable binding partner of PSGL-1 and a suitable binding partner of LFA-1 are combined to identify T cells that express both LFA-1 and PSGL-1.

A respective binding partner of e.g. CD62L, PSGL-1, LFA-1 or CD3, as well as a binding partner for another selected cell-characteristic protein, may be an immunoglobulin, a fragment thereof or a proteinaceous binding molecule with immunoglobulin-like functions. An antibody fragment generally contains an antigen binding or variable region. Examples of (recombinant) antibody fragments are immunoglobulin fragments such as Fab fragments, Fab′ fragments, Fv fragments, single-chain Fv fragments (scFv), diabodies or domain antibodies (Holt, L. J., et al., Trends Biotechnol. (2003), 21, 11, 484-490). An example of a proteinaceous binding molecule with immunoglobulin-like functions is a mutein based on a polypeptide of the lipocalin family (WO 03/029462, Beste et al., Proc. Natl. Acad. Sci. USA (1999) 96, 1898-1903). Lipocalins, such as the bilin binding protein, the human neutrophil gelatinase-associated lipocalin, human Apolipoprotein D or glycodelin, posses natural ligand-binding sites that can be modified so that they bind to selected small protein regions known as haptens. Examples of other proteinaceous binding molecules are the so-called glubodies (see e.g. international patent application WO 96/23879 or Napolitano, E. W., et al., Chemistry & Biology (1996) 3, 5, 359-367), proteins based on the ankyrin scaffold (Mosavi, L. K., et al., Protein Science (2004) 13, 6, 1435-1448) or crystalline scaffold (e.g. internation patent application WO 01/04144), the proteins described in Skerra, J. Mol. Recognit. (2000) 13, 167-187, AdNectins, tetranectins and avimers. Avimers contain so called A-domains that occur as strings of multiple domains in several cell surface receptors (Silverman, J., et al., Nature Biotechnology (2005) 23, 1556-1561). Adnectins, derived from a domain of human fibronectin, contain three loops that can be engineered for immunoglobulin-like binding to targets (Gill, D. S. & Damle, N. K., Current Opinion in Biotechnology (2006) 17, 653-658). Tetranectins, derived from the respective human homotrimeric protein, likewise contain loop regions in a C-type lectin domain that can be engineered for desired binding (ibid.). Peptoids, which can act as protein ligands, are oligo(N-alkyl) glycines that differ from peptides in that the side chain is connected to the amide nitrogen rather than the [symbol] carbon atom. Peptoids are typically resistant to proteases and other modifying enzymes and can have a much higher cell permeability than peptides (see e.g. Kwon, Y.-U., and Kodadek, T., J. Am. Chem. Soc. (2007) 129, 1508-1509). A suitable antibody may in some embodiments also be a multispecific antibody that includes several immunoglobulin fragments.

An immunoglobulin or a proteinaceous binding molecule with immunoglobulin-like functions may be PEGylated or hyperglycosylated if desired. In some embodiments a proteinaceous binding molecule with immunoglobulin-like functions is a fusion protein of one of the exemplary proteinaceous binding molecules above and an albumin-binding domain, for instance an albumin-binding domain of streptococcal protein G. In some embodiments a proteinaceous binding molecule with immunoglobulin-like functions is a fusion protein of an immunoglobulin fragment, such as a single-chain diabody, and an immunoglobulin binding domain, for instance a bacterial immunoglobulin binding domain. As an illustrative example, a single-chain diabody may be fused to domain B of staphylococcal protein A as described by Unverdorben et al. (Protein Engineering, Design & Selection [2012] 25, 81-88).

A molecule that forms a complex with a binding partner of e.g. CD62L, PSGL-1, LFA-1 or CD4 may likewise be an immunoglobulin, a fragment thereof or a proteinaceous binding molecule with immunoglobulin-like functions, as explained above. Thus, in an exemplary embodiment detecting the amount of CD62L, e.g. on a cell surface, may carried out using a first antibody or antibody fragment capable of specifically binding CD62L, as well as a second antibody or antibody fragment capable of specifically binding the first antibody or antibody fragment.

An immunoglobulin may be monoclonal or polyclonal. The term “polyclonal” refers to immunoglobulins that are heterogenous populations of immunoglobulin molecules derived from the sera of animals immunized with an antigen or an antigenic functional derivative thereof. For the production of polyclonal immunoglobulins, one or more of various host animals may be immunized by injection with the antigen. Various adjuvants may be used to increase the immunological response, depending on the host species. “Monoclonal immunoglobulins”, also called “monoclonal antibodies”, are substantially homogenous populations of immunoglobulins to a particular antigen.

They may be obtained by any technique which provides for the production of immunoglobulin molecules by continuous cell lines in culture. Monoclonal immunoglobulins may be obtained by methods well known to those skilled in the art (see for example, Köhler et al., Nature (1975) 256, 495-497, and U.S. Pat. No. 4,376,110). An immunoglobulin or immunoglobulin fragment with specific binding affinity only for e.g. CD62L, PSGL-1, CD3, LFA-1, CD8 or CD4 can be isolated, enriched, or purified from a prokaryotic or eukaryotic organism. Routine methods known to those skilled in the art enable production of both immunoglobulins or immunoglobulin fragments and proteinaceous binding molecules with immunoglobulin-like functions, in both prokaryotic and eukaryotic organisms.

In more detail, an immunoglobulin may be isolated by comparing its binding affinity to a protein of interest, e.g. L-selectin, with its binding affinity to other polypeptides. Humanized forms of the antibodies of the present invention may be generated using one of the procedures known in the art such as chimerization or CDR grafting. In general, techniques for preparing monoclonal antibodies and hybridomas are well known in the art. Any animal such as a goat, a mouse or a rabbit that is known to produce antibodies can be immunized with the selected polypeptide, e.g. L-selectin. Methods for immunization are well known in the art. Such methods include subcutaneous or intraperitoneal injection of the polypeptide. One skilled in the art will recognize that the amount of polypeptide used for immunization and the immunization regimen will vary based on the animal which is immunized, including the species of mammal immunized, its immune status and the body weight of the mammal, as well as the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or β-galactosidase) or through the inclusion of an adjuvant during immunization.

Typically, the immunized mammals are bled and the serum from each blood sample is analysed for particular antibodies using appropriate screening assays. As an illustrative example, anti-CD62L, anti-PSGL-1 or anti-LFA-1 immunoglobulins may be identified by immunoprecipitation of ¹²⁵I-labeled cell lysates from CD62L, PSGL-1 or LFA-1-expressing cells. Anti-CD62L, PSGL-1 or anti-LFA-1 immunoglobulins may also be identified by flow cytometry, e.g., by measuring fluorescent staining of Ramos cells incubated with an immunoglobulin believed to recognize CD62L, PSGL-1 or LFA-1, as applicable.

For monoclonal immunoglobulins, lymphocytes, typically splenocytes, from the immunized animals are removed, fused with an immortal cell line, typically myeloma cells, such as SP2/0-Ag14 myeloma cells, and allowed to become monoclonal immunoglobulin producing hybridoma cells. Typically, the immortal cell line such as a myeloma cell line is derived from the same mammalian species as the lymphocytes. Illustrative immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion may then be selected using HAT medium, which kills unfused and unproductively fused myeloma cells (unfused splenocytes die after several days because they are not transformed).

Any one of a number of methods well known in the art can be used to identify a hybridoma cell which produces an immunoglobulin with the desired characteristics. Typically the culture supernatants of the hybridoma cells are screened for immunoglobulins against the antigen. Suitable methods include, but are not limited to, screening the hybridomas with an ELISA assay, Western blot analysis, or radioimmunoassay. Hybridomas prepared to produce anti-CD62L, anti-PSGL-1 or anti-LFA-1 immunoglobulins may for instance be screened by testing the hybridoma culture supernatant for secreted antibodies having the ability to bind to a recombinant CD62L, PSGL-1 or LFA-1 expressing cell line. To produce antibody homologs which are within the scope of the invention, including for example, anti-CD62L, PSGL-1 or anti-LFA-1 antibody homologs, that are intact immunoglobulins, hybridoma cells that tested positive in such screening assays can be cultured in a nutrient medium under conditions and Dora time sufficient to allow the hybridoma cells to secrete the monoclonal immunoglobulins into the culture medium. Tissue culture techniques and culture media suitable for hybridoma cells are well known in the art. The conditioned hybridoma culture supernatant may be collected and for instance the anti-CD62L immunoglobulins or the anti-PSGL-1 immunoglobulins optionally further purified by well-known methods. Alternatively, the desired immunoglobulins may be produced by injecting the hybridoma cells into the peritoneal cavity of an unimmunized mouse. The hybridoma cells proliferate in the peritoneal cavity, secreting the immunoglobulin which accumulates as ascites fluid. The immunoglobulin may be harvested by withdrawing the ascites fluid from the peritoneal cavity with a syringe.

Hybridomas secreting the desired immunoglobulins are cloned and the class and subclass are determined using procedures known in the art. For polyclonal immunoglobulins, an immunoglobulin containing antiserum is isolated from the immunized animal and is screened for the presence of immunoglobulins with the desired specificity using one of the above-described procedures. The above-described antibodies, including immunoglobulins, may also be immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art.

A plurality of conventional display technologies is available to select an immunoglobulin, immunoglobulin fragment or proteinaceous binding molecule. Li et al. (Organic & Biomolecular Chemistry (2006), 4, 3420-3426) have for example demonstrated how a single-chain Fv fragment capable of forming a complex with a selected DNA adapter can be obtained using phage display. Display techniques for instance allow the generation of engineered immunoglobulins and ligands with high affinities for a selected target molecule. It is thus also possible to display an array of peptides or proteins that differ only slightly, typically by way of genetic engineering. Thereby it is possible to screen and subsequently evolve proteins or peptides in terms of properties of interaction and biophysical parameters. Iterative rounds of mutation and selection can be applied on an in vitro basis.

In vitro display technology for the selection of peptides and proteins relies on a physical linkage between the peptide or protein and a nucleic acid encoding the same. A large panel of techniques has been established for this purpose, with the most commonly used being phage/virus display, ribosome display, cell-surface display, ‘peptides on plasmids’, mRNA display, DNA display, and in vitro compartmentalisation including micro-bead display (for reviews see e.g. Rothe, A., et al., FASEB J. (2006) 20, 1599-1610; Sergeeva, A., et al., Advanced Drug Delivery Reviews (2006) 58, 1622-1654).

Different means of physically linking a peptide, including a protein, and a nucleic acid are also available. Expression in a cell with a cell surface molecule, expression as a fusion polypeptide with a viral/phage coat protein, a stabilised in vitro complex of an RNA molecule, the ribosome and the respective polypeptide, covalent coupling in vitro via a puromycin molecule or via micro-beads are examples of ways of linking the protein/peptide and the nucleic acid presently used in the art. A further display technique relies on a water-in-oil emulsion. The water droplets serve as compartments in each of which a single gene is transcribed and translated (Tawfik, D. S., & Griffiths, A. D., Nature Biotech. (1998) 16, 652-656, US patent application 2007/0105117). This physical linkage between the peptide including the protein, and the nucleic acid (encoding it) provides the possibility of recovering the nucleic acid encoding the selected peptide/protein. Compared to techniques such as immunoprecipitation, in display techniques thus not only binding partners of a selected target molecule can be identified or selected, but the nucleic acid of this binding partner can be recovered and used for further processing. Present display techniques thus provide means for e.g. target discovery, lead discovery and lead optimisation. Vast libraries of peptides or proteins, e.g. antibodies, potentially can be screened on a large scale.

Illustrative examples of antibodies such as immunoglobulins that specifically bind to PSGL-1 have for example been disclosed in international patent application WO 2005/110475. Examples of immunoglobulins and immunoglobulin fragments that specifically bind to conformational epitopes of PSGL-1 have for example been disclosed in international patent application WO 2012/088265.

As indicated above, a detectable marker may be coupled to a binding partner of CD62L, of PSGL-1, of LFA-1, of CD4, of CD8 or CD3, as the case may be, or a molecule that forms a complex with the binding partner of CD62L, PSGL-1, LFA-1, CD4, CD8 or CD3. A respective detectable marker, which may be coupled to a binding partner of CD62L, PSGL-1, LFA-1, CD4, CD8 or CD3, or a molecule that forms a complex therewith, may be an optically detectable label, a fluorophore, or a chromophore. Examples of suitable labels include, but are not limited to, an organic molecule, an enzyme, a radioactive, fluorescent, and/or chromogenic moiety, a luminescent moiety, a hapten, digoxigenin, biotin, a metal complex, a metal and colloidal gold. Accordingly an excitable fluorescent dye, a radioactive amino acid, a fluorescent protein or an enzyme may for instance be used to detect e.g. the level of CD62L or the level of PSGL-1. Examples of suitable fluorescent dyes include, but are not limited to, fluorescein isothiocyanate, 5,6-carboxymethyl fluorescein, Cascade Blue®, Oregon Green®, Texas red, nitrobenz-2-oxa-1,3-diazol-4-yl, coumarin, dansyl chloride, rhodamine, amino-methyl coumarin, DAPI, Eosin, Erythrosin, BODIPY®, pyrene, lissamine, xanthene, acridine, an oxazine, phycoerythrin, a Cy dye such as Cy3, Cy3.5, Cy5, Cy5PE, Cy5.5, Cy7, Cy7PE or Cy7APC, an Alexa dye such as Alexa 647, and NBD (Naphthol basic dye). Examples of suitable fluorescent protein include, but are not limited to, EGFP, emerald, EYFP, a phycobiliprotein such as phycoerythrin (PE) or allophycocyanin, Monomeric Red Fluorescent Protein (mRFP), mOrange, mPlum and mCherry. In some embodiments a reversibly photoswitchable fluorescent protein such as Dronpa, bsDronpa and Padron may be employed (Andresen, M., et al., Nature Biotechnology (2008) 26, 9, 1035). Regarding suitable enzymes, alkaline phosphatase, soybean peroxidase, or horseradish peroxidase may serve as a few illustrative examples. In some embodiments a method of detection may include electrophoresis, HPLC, flow cytometry, fluorescence correlation spectroscopy or a modified form of these techniques. Some or all of these steps may be part of an automated separation/detection system.

In some embodiments the binding partner of e.g. CD62L, PSGL-1, LFA-1 or CD3, as well as a binding partner for another selected cell-characteristic protein, further includes a capture molecule. Such a capture molecule allows immobilization of the binding partner, and thereby also of a complex formed between e.g. CD62L, PSGL-1, LFA-1 or CD3, or another selected cell-characteristic protein, on a surface or on a polymeric molecule, including an immunoglobulin, an immunoglobulin fragment or a proteinaceous binding molecule with immunoglobulin-like functions. A respective surface may for instance be the surface of a micro- or nanoparticle, the surface of a container or the surface of a particularly designed device used for presentation purposes during measurement. A micro- or nanoparticle may in some embodiments include, essentially consist of or consist of a metal, a metalloid or a polymer. In some embodiments the micro- or nanoparticle is magnetic, such as paramagnetic or supermagnetic. The capture molecule may be immobilised on the surface via a covalent bond or a non-covalent bond.

The capture molecule has an affinity to a binding partner of the capture molecule and is capable of forming a complex with the binding partner of the capture molecule. Hence, the capture molecule and the binding partner of the capture molecule define a specific binding pair. Accordingly, a pair of capture molecule and binding partner of the capture molecule may be selected as desired, for example according to the binding partner of CD62L, PSGL-1, LFA-1 or CD3 or to the measurement conditions used in detection of for instance CD62L. Examples of a capture molecule include, but are not limited to, a nucleic acid molecule, an oligonucleotide, a protein, an oligopeptide, a polysaccharide, an oligosaccharide, a synthetic polymer, a drug candidate molecule, a drug molecule, a drug metabolite, a metal ion, and a vitamin. Three illustrative examples of suitable capture molecule are biotin, dinitrophenol or digoxigenin. Where the binding partner of the capture molecule is a protein/polypeptide, or a peptide, further examples of a capture molecule include, but are not limited to, a streptavidin binding tag such as the STREP-TAGS® described in US patent application US 2003/0083474, U.S. Pat. No. 5,506,121 or 6,103,493, an immunoglobulin domain, maltose-binding protein, glutathione-S-transferase (GST), calmodulin binding peptide (CBP), FLAG-peptide (e.g. of the sequence Asp-Tyr-Lys-Asp-Asp-Asp-Asp-Lys-Gly), the T7 epitope (Ala-Ser-Met-Thr-Gly-Gly-Gln-Gln-Met-Gly), maltose binding protein (MBP), the HSV epitope of the sequence Gln-Pro-Glu-Leu-Ala-Pro-Glu-Asp-Pro-Glu-Asp of herpes simplex virus glycoprotein D, the Vesicular Stomatitis Virus Glycoprotein (VSV-G) epitope of the sequence Tyr-Thr-Asp-Ile-Glu-Met-Asn-Arg-Leu-Gly-Lys, the hemagglutinin (HA) epitope of the sequence Tyr-Pro-Tyr-Asp-Val-Pro-Asp-Tyr-Ala and the “myc” epitope of the transcription factor c-myc of the sequence Glu-Gln-Lys-Leu-Ile-Ser-Glu-Glu-Asp-Leu. Where the binding partner of the capture molecule is a nucleic acid, a polynucleotide or an oligonucleotide, a capture molecule may furthermore be an oligonucleotide. Such an oligonucleotide tag may for instance be used to hybridize to an immobilised oligonucleotide with a complementary sequence.

As an illustrative example, the capture molecule may be a metal ion bound by a respective metal chelator, such as ethylenediamine, ethylenediaminetetraacetic acid (EDTA), ethylene glycol tetraacetic acid (EGTA), diethylenetriaminepentaacetic acid (DTPA), N,N-bis(carboxymethyl)glycine (also called nitrilotriacetic acid, NTA), 1,2-bis(o-aminophenoxy)ethane-N,N,N′,N′-tetraacetic acid (BAPTA), 2,3-dimercapto-1-propanol (dimmercaprol), porphine or heme. A respective metal ion may define a receptor molecule for a peptide of a defined sequence, which may also be included in a protein. In line with the standard method of immobilised metal affinity chromatography used in the art, for example an oligohistidine tag of a respective peptide or protein is capable of forming a complex with copper (Cu²⁺), nickel (Ni²⁺), cobalt (Co²⁺), or zinc (Zn²⁺) ions, which can for instance be presented by means of the chelator nitrilotriacetic acid (NTA).

The capture molecule may be immobilised on a surface (vide infra) such as the surface of a particle such as a metal containing bead. The capture molecule may be immobilised by any means. It may be immobilised on a portion or the entire area of a surface. An illustrative example is the mechanical spotting of a nucleic acid capture molecule onto a metal surface. This spotting may be carried out manually, e.g. by means of a pipette, or automatically, e.g. by means of a micro robot. As an illustrative example, a protein capture molecule, a peptide capture molecule or the polypeptide backbone of a PNA capture molecule may be covalently linked to a gold surface via a thio-ether-bond.

In embodiments where both the capture molecule and the binding partner of a biomarker are a nucleic acid molecule, including an oligonucleotide, the capture molecule typically has a nucleotide sequence that is at least partially complementary to a portion of a strand of the binding partner of the capture molecule. As a further illustrative example, Avidin or streptavidin may be employed to immobilise a biotinylated nucleic acid, or a biotin containing monolayer of gold may be employed (Shumaker-Parry, J. S., et al., Anal. Chem. (2004) 76, 918). As another illustrative example, the capture molecule may be a metal ion bound by a respective metal chelator (see above).

As explained above, a binding partner can bind a nucleic acid molecule, a peptide, a protein, a saccharide, a polysaccharide or a lipid. In some embodiments the binding partner is a PNA molecule. As indicated above, a PNA molecule is a nucleic acid molecule in which the backbone is a pseudopeptide rather than a sugar. Accordingly, PNA generally has a charge neutral backbone, in contrast to DNA or RNA. Nevertheless, PNA is capable of hybridising at least complementary and substantially complementary nucleic acid strands, just as e.g. DNA or RNA (to which PNA is considered a structural mimic). In some embodiments the binding partner is an aptamer, including a Spiegelmer®, described in e.g. WO 01,92655. An aptamer is typically a nucleic acid molecule that can be selected from a random nucleic acid pool based on its ability to bind a selected other molecule such as a peptide, a protein, a nucleic acid molecule a or a cell. Aptamers, including Spiegelmers, are able to bind molecules such as peptides, proteins and low molecular weight compounds. Spiegelmers® are composed of L-isomers of natural oligonucleotides. Aptamers are engineered through repeated rounds of in vitro selection or through the SELEX (systematic evolution of ligands by exponential enrichment) technology. The affinity of Spiegelmers to their target molecules often lies in the pico- to nanomolar range and is thus comparable to immunoglobulins. An aptamer may also be a peptide. A peptide aptamer consists of a short variable peptide domain, attached at both ends to a protein scaffold.

In typical embodiments the binding partner is an immunoglobulin or a proteinaceous binding molecule with immunoglobulin-like functions as defined above. In some embodiments the binding partner may be detectably labelled as explained above, for example where the binding partner is intended to be used together with a detection agent that binds to the biomarker and/or the binding partner. The binding partner and/or a respective detection agent may be detectably labeled by linking the same, typically covalently, to a detectable marker such as a radioactive label, a fluorescent moiety, a chemical entity of low molecular weight, an oligonucleotide, an enzyme, or a protein such as a fluorescent protein such as a Green Fluorescent Protein (cf. above). It is understood that the method may also include any molecules which can be used to indirectly indicate the level of the target molecule of interest such as CD62L, PSGL-1, CD3, CD4, CD8, CD18 or CD11a. The binding partner may in some embodiments be an immunoglobulin, a portion thereof, a proteinaceous binding molecule with immunoglobulin-like functions, a receptor for the biomarker or a portion thereof or a ligand for the biomarker or a portion thereof. The detection agent may in some embodiments be an immunoglobulin, a portion thereof, a proteinaceous binding molecule with immunoglobulin-like functions, a receptor for the biomarker or a portion thereof, a ligand for the biomarker or a portion thereof or a binding partner binding partner or a portion thereof.

In some embodiments a binding partner capable of binding a particular target nucleic acid molecule such as an mRNA molecule encoding e.g. CD62L, PSGL-1, CD18 or CD11a, is a nucleic acid molecule that includes a nucleotide sequence that is at least partially complementary to a portion of a strand of such a target nucleic acid molecule. A nucleotide sequence is the complement of another nucleotide sequence if all of the nucleotides of the first sequence are complementary to all of the nucleotides of the second sequence. Accordingly, the respective nucleotide sequence will specifically hybridise to, or undergo duplex formation with, the respective portion of the target nucleic acid molecule under suitable hybridisation assay conditions, in particular of ionic strength and temperature.

As an illustrative example, a single-stranded nucleic acid molecule may be selected as a nucleic acid binding partner. Such a single-stranded nucleic acid molecule may have a nucleic acid sequence that is at least partially complementary to at least a portion of a strand of the target nucleic acid molecule. The respective nucleotide sequence of the nucleic acid binding partner may for example be 70, for example 80 or 85, including 100% identical to another nucleic acid sequence. The higher the percentage to which the two sequences are complementary to each other (i.e. the lower the number of mismatches), the higher is typically the sensitivity of the method of the invention. In typical embodiments the respective nucleotide sequence is substantially complementary to at least a portion of the target nucleic acid molecule. “Substantially complementary” as used in this document refers to the fact that a given nucleic acid sequence is at least 90% identical to another nucleic acid sequence. A substantially complementary nucleic acid sequence is in some embodiments 95%, such as 100% identical to another nucleic acid sequence. The term “complementary” or “complement” refers to two nucleotides that can form multiple favourable interactions with one another. Such favourable interactions are specific association between opposing or adjacent pairs of nucleic acid (including nucleic acid analogue) strands via matched bases, and include Watson-Crick base pairing. As an illustrative example, in two given nucleic acid molecules (e.g. DNA molecules) the base adenosine is complementary to thymine or uracil, while the base cytosine is complementary to guanine. A nucleic acid probe used in the context of the present invention may be used to probe the sample by usual hybridization methods to detect the presence of nucleic acid molecules encoding e.g. CD62L, PSGL-1, CD18 or CD11a.

Interactions between two or more nucleic acid molecules are generally sequence driven interactions referred to as hybridization. Sequence driven interaction is an interaction that occurs between two nucleotides or nucleotide analogs or nucleotide derivatives in a nucleotide specific manner (supra). Typically sequence driven interactions occur on the Watson-Crick face or Hoogsteen face of the respective nucleotide. The hybridization of two nucleic acids is affected by a number of conditions and parameters known to those skilled in the art. For example, the salt concentrations, pH, and temperature of the reaction all affect whether two nucleic acid molecules will hybridize. For example, concentrations, pH, and temperature of the reaction all affect whether two nucleic acid molecules will hybridize. For example, in some embodiments selective hybridization conditions can be defined as stringent hybridization conditions. For example, stringency of hybridization is controlled by both temperature and salt concentration of either or both of the hybridization and washing steps. For example, conditions of hybridization that achieve selective interactions between complementary sequences may involve hybridization in high ionic strength solution (6×SSC or 6×SSPE) at a temperature that is in the range from about 12 to about 25° C. below the Tm, the melting temperature at which half of the molecules dissociate from their hybridization partners, followed by washing at a combination of temperature and salt concentration chosen so that the washing temperature is in the range from about 5° C. to about 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations than for DNA-DNA hybridizations.

In order to obtain nucleic acid probes having nucleotide sequences which correspond to altered portions of the amino acid sequence of the polypeptide of interest, chemical synthesis can be carried out. The synthesized nucleic acid probes may be first used as primers in a polymerase chain reaction (PCR) carried out in accordance with recognized PCR techniques, essentially according to standard PCR protocols utilizing the appropriate template, in order to obtain the probes that can be used in the context of the present invention.

One skilled in the art will readily be able to design such probes based on a sequence as referred to herein using methods of computer alignment and sequence analysis well known in the art. As explained above, a respective hybridization probe can be labeled by standard labeling techniques using a detectable marker, such as with a radiolabel, enzyme label, fluorescent label, biotin-avidin label, or chemiluminescence (supra). After hybridization, the probes may be visualized using known methods. A nucleic acid probe may be immobilized on a solid support. Examples of such solid supports include, but are not limited to, plastics such as polycarbonate, complex carbohydrates such as agarose and sepharose, and acrylic resins, such as polyacrylamide and latex beads. As an illustrative example one or more nucleic acid probes may be bound to or immobilized on a solid support. The solid support may be a chip, for example a DNA microchip. Techniques for coupling nucleic acid probes to such solid supports are well known in the art.

The most frequently used methods for determining the concentration of nucleic acids include the detection by autoradiography, fluorescence, chemiluminescence or bioluminescence as well as electrochemical and electrical techniques. A further suitable technique is the electrical detection of a target nucleic acid molecule as disclosed in international patent applications WO 2009/041917 and WO 2008/097190, both being incorporated herein by reference in their entirety. In case of conflict, the present specification, including definitions, will control. A technique for the specific detection of a selected nucleic acid well established in the art is based on the hybridisation between a nucleic acid binding partner and a target nucleic acid. Typically the respective nucleic acid binding partner is immobilised onto a solid support, and subsequently one of the above mentioned detection methods is employed.

As indicated above, an immunoglobulin labeled with a fluorescence dye may for instance be used to optically detect the presence of a certain protein or polypeptide. Nucleic acid intercalating dyes, such as YOYO, JOJO, BOBO, POPO, TOTO, LOLO, SYBR, SYTO, SYTOX, PicoGreen, or Oligreen as available from Molecular Probes, may be used for optical detection.

In some embodiments determining the level of expression of the gene of interest includes determining the level of transcription into mRNA. RNA encoding the protein of interest in the sample, such as CD62L, PSGL-1, CD11A, CD18, CD3, CD4 or CD8 may be amplified using any available amplification technique, such as polymerase chain reaction (PCR), including multiplex PCR, nested PCR and amplification refractory mutation specific (ARMS) PCR (also called allele-specific PCR (AS-PCR), rolling circle amplification (RCA), nucleic acid sequence based amplification (NASBA), ligase chain reaction (LCR), QB replicase chain reaction, loop-mediated isothermal amplification (LAMP), transcription mediated amplification (TMA) and strand displacement amplification (SDA), including genome strand displacement amplification (WGSDA), multiple strand displacement amplification (MSDA), and gene specific strand displacement amplification (GS-MSDA). Detection of the obtained amplification products may be performed in numerous ways known in the art. Examples include, but are not limited to, electrophoretic methods such as agarose gel electrophoresis in combination with a staining such as ethidium bromide staining. In other embodiments the method of the invention is accompanied by real time detection, such as real time PCR. In these embodiments the time course of the amplification process is monitored. A means of real time detection commonly used in the art involves the addition of a dye before the amplification process. An example of such a dye is the fluorescence dye SYBR Green, which emits a fluorescence signal only when bound to double-stranded nucleic acids.

As explained above, typically a detectable label or marker is used. Such a marker or label may be included in a nucleic acid that includes the sequence to be amplified. A marker may also be included in a primer or a probe. It may also be incorporated into the amplification product in the course of the reaction. In some embodiments such a marker compound, e.g. included in a nucleic acid, is an optically detectable label, a fluorophore, or a chromophore. An illustrative example of a marker compound is 6-carboxyfluorescein (FAM).

As an illustrative example, real-time PCR may be used to determine the level of RNA encoding the protein of interest in the sample, such as CD62L, PSGL-1, CD11A, CD18, CD3, CD4 or CD8. Such a PCR procedure is carried out under real time detection, so that the time course of the amplification process is monitored. PCR is characterised by a logarithmic amplification of the target sequences. For the amplification of RNA, a reverse transcriptase-PCR is used. Design of the primers and probes required to detect expression of a biomarker of the invention is within the skill of a practitioner of ordinary skill in the art. In some embodiments RNA from the sample is isolated under RNAse free conditions and then converted to DNA via the use of a reverse transcriptase. Reverse transcription may be performed prior to RT-PCR analysis or simultaneously, within a single reaction vessel. RT-PCR probes are oligonucleotides that have a fluorescent moiety, also called reporter dye, attached to the 5′ end and a quencher moiety coupled to the 3′ end (or vice versa). These probes are typically designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. Thereby the fluorescent and quenching moieties are decoupled. Fluorescence increases then in each cycle, in a manner proportional to the amount of probe cleavage. Fluorescence signal emitted from the reaction can be measured or followed over time using equipment which is commercially available using routine and conventional techniques. Quantitation of biomarker RNA in a sample being evaluated may be performed by comparison of the amplification signal to that of one or more standard curves where known quantities of RNA were evaluated in a similar manner. In some embodiments, the difference in biomarker expression is measured as the difference in PCR cycle time to reach a threshold fluorescence, or “dCT.”

As indicated above, in some embodiments T cells such as CD3⁺ T cells are isolated by means of a magnetic, such as paramagnetic or supermagnetic surface. In some embodiments CD4⁺ T cells and/or CD8⁺ T cells may be isolated by means of a magnetic surface. Such a surface may for instance be the surface of a micro- or nanoparticle (supra). Typically a respective surface has covalently or non-covalently bound binding partner such as antibodies coupled onto it. In some embodiments monosized magnetic particles as available from Life Technologies can be used. In some embodiments the technique of magnetic-activated cell sorting (MACS') may be employed. In this technique complexes formed of T cells and magnetic particles are loaded onto a column placed in a strong magnetic field. While other matter passes through the column, complexes of the magnetic particles and T cells remain due to the action of the magnetic field. Likewise, in some embodiments T cells, including CD3⁺ T cells, CD4⁺ T cells and/or CD8⁺ T cells, are isolated using a flow cytometry based method, such as fluorescence-activated cell sorting (FACS), a method further explained below. Cell sorting may be automated using a variety of technologies. For example, one or more steps may be initiated, or cell sorting parameters may be adjusted, using a series of computer executable instructions residing on a suitable computer readable medium. As an illustrative example, computer executable instructions may control a switching element that may be configured to turn the delivery of cells into the measurement “on” or “off”.

In some embodiments the level or amount of CD62L, PSGL-1, LFA-1 and/or CD3 on the surface of cells in the sample is determined using a flow cytometry based analysis. Such an embodiment of a method or use of the invention may be taken to define a method of performing flow cytometry. Flow cytometry based analysis is typically combined with optical detection to identify and classify cells. This allows speed, selectivity/specificity, and a non-invasive nature of the technique. Typically fluorescent markers are used, which are compounds that bind to specific structures or molecules on the surface or within target cells. Such fluorescent markers are introduced into the mixture of cells, whereafter the mixture is rinsed to remove excess fluorescent markers. In some embodiments flow cytometry is combined with immunofluorescence.

Immunofluorescence is generally achieved using a binding partner as described above, which is linked to, or includes, a fluorophore as a detectable marker (supra). Flow cytometry is a technique for counting, examining, and sorting microscopic particles such as biological cells suspended in a stream of fluid. It allows a simultaneous multiparametric analysis of the physical and chemical characteristics of single cells flowing through an optical or electronic detection device. An illustrative example of a well established flow cytometry based analysis in the art is FACS. FACS allows sorting a heterogeneous mixture of cells into a plurality of containers, one cell at a time, based upon the specific light scattering and fluorescent characteristics of each cell. Thereby FACS allows the sorting of subpopulations of cells of interest and their further use in in vitro and in vivo assays. FACS is often used in combination with monoclonal immunoglobulins as a reagent to detect cells as having a particular antigen, indicative of an expressed protein (supra).

This technique allows the concurrent fast, objective and quantitative recording of fluorescent signals from individual cells and the physical separation of respective cells according to particular interest. Fluorescent signals used in flow cytometry, for instance when quantifying and/or sorting cells by any marker present on or in the cell, are typically fluorescently-tagged antibody preparations or fluorescently-tagged ligands for binding to antibodies or other antigen-, epitope- or ligand-specific agent, such as with biotin/avidin binding systems or fluorescently-labeled and optionally addressable beads (e.g. LUMINEX® microspheres). Depending of the equipment used, any desired detectable marker or combination of detectable markers can be detected by the optics and/or electronics of a flow cytometer. Current three-laser, “multidimensional”, FACS machines enable up to 14 simultaneous single-cell measurements, such as two light scatter detectors and 12 fluorescence plus forward detectors allowing for example the detection of fluorescent surface/intracellular markers. As an illustrative example, the three lasers of a FACS machine may be a krypton laser operating at 407 nm, an argon laser operating at 488 nm, and a dye laser operating at 595 nm.

The FACS technique has been used extensively in relation to antigens expressed on the surface of cells, including cells that remain alive during, and after, FACS. Similarly, the method has been used with intracellular reporter gene systems based on the expression of a detectably labeled gene product by the cell. Accordingly, the technique not only allows detecting the presence of e.g. CD62L, PSGL-1, LFA-1, CD4 or CD8 on the cell surface, but also detecting the presence of RNA or DNA within the cell, for example RNA encoding CD62L, PSGL-1 and CD3 or CD4 (vide infra). Therefore FACS can also be used to determine the amount of nucleic acid formation from the SELL gene, which encodes CD62L, in cells, such as T cells, including CD4⁺ T cells or CD8⁺ T cells, of the sample from the subject.

In some embodiments determining the amount of CD62L, PSGL-1, LFA-1, CD3, CD4 and/or CD8 on the surface of cells in the sample is carried out by determining the amount of CD62L, PSGL-1, LFA-1, CD4 and/or CD8 that is accessible in the sample. Such a method can be taken to be a method of determining extracellular CD62L, PSGL-1, LFA-1, CD3, CD4 and/or CD8 in the sample. In embodiments where cells such as T cells are immobilized on a surface, for example using a capture reagent as detailed above, before determining the amount of e.g. CD62L, PSGL-1, LFA-1 and/or CD3, any soluble LFA-1, PSGL-1, CD62L and/or CD3, i.e. LFA-1, PSGL-1, CD62L and/or CD3 that is not immobilized on the surface of a cell, can easily be removed, for example by way of washing. In such embodiments therefore only CD62L, PSGL-1, LFA-1 and/or CD3 on the surface of cells is being determined. An illustrative example of a suitable technique in this regard is a radiolabel assay such as a Radioimmunoassay (RIA) or an enzyme-immunoassay such as an Enzyme Linked Immunoabsorbent Assay (ELISA). While a RIA is based on the measurement of radioactivity associated with a complex formed between an immunoglobulin or a proteinaceous binding molecule with immunoglobulin-like functions and an antigen, an ELISA is based on the measurement of an enzymatic reaction associated with a complex formed between an immunoglobulin or a proteinaceous binding molecule with immuneglobulin-like functions and an antigen. Typically a radiolabel assay or an enzyme-immunoassay involves one or more separation steps in which a binding partner of e.g. CD62L, PSGL-1, LFA-1 or CD3 that has not formed a complex with CD62L, PSGL-1, LFA-1 or CD3 is being removed, thereby leaving only binding partner of CD62L, PSGL-1, LFA-1 or CD3 behind, which has formed a complex with CD62L, PSGL-1, LFA-1 or CD3. This allows the generation of specific signals originating from the presence of CD62L, PSGL-1, LFA-1 or CD3.

An ELISA or RIA test can be competitive for measuring the amount of CD62L, PSGL-1, LFA-1, CD3, CD4 and/or CD8, i.e. the amount of antigen. For example, an enzyme labeled antigen is mixed with a test sample containing antigen, which competes for a limited amount of immunoglobulin or a proteinaceous binding molecule with immunoglobulin-like functions. The reacted (bound) antigen is then separated from the free material, and its enzyme activity is estimated by addition of substrate. An alternative method for antigen measurement is the double immunoglobulin/proteinaceous binding molecule sandwich technique. In this modification a solid phase is coated with specific immunoglobulin or a proteinaceous binding molecule with immunoglobulin-like functions. This is then reacted with the sample from the subject that contains the antigen. Then enzyme labeled specific immunoglobulin/proteinaceous binding molecule is added, followed by the enzyme substrate. The ‘antigen’ in the test sample is thereby ‘captured’ and immobilized on to the sensitized solid phase where it can itself then immobilize the enzyme labeled immunoglobulin/proteinaceous binding molecule. This technique is analogous to the immunoradiometric assays.

In an indirect ELISA method, an antigen is immobilized by passive adsorption on to the solid phase. A test serum may then be incubated with the solid phase and any immunoglobulin in the test serum forms a complex with the antigen on the solid phase. Similarly a solution of a proteinaceous binding molecule with immunoglobulin-like functions may be incubated with the solid phase to allow the formation of a complex between the antigen on the solid phase and the proteinaceous binding molecule. After washing to remove unreacted serum components an immunoglobulin or proteinaceous binding molecule with immunoglobulin-like functions, linked to an enzyme is contacted with the solid phase and incubated. Where the second reagent is selected to be a proteinaceous binding molecule with immunoglobulin-like functions, a respective proteinaceous binding molecule that specifically binds to the proteinaceous binding molecule or the immunoglobulin directed against the antigen is used. A complex of the second proteinaceous binding molecule or immunoglobulin and the first proteinaceous binding molecule or immunoglobulin, bound to the antigen, is formed. Washing again removes unreacted material. In the case of RIA radioactivity signals are being detected. In the case of ELISA the enzyme substrate is added. Its colour change will be a measure of the amount of the immobilized complex involving the antigen, which is proportional to the antibody level in the test sample.

In another embodiment the immunoglobulin or the proteinaceous binding molecule with immunoglobulin-like functions may be immobilized onto a surface, such as the surface of a polymer bead (supra), or coated onto the surface of a device such as a polymer plate or a glass plate. As a result the immune complexes can easily be separated from other components present by simply washing the surface, e.g. the beads or plate. This is the most common method currently used in the art and is referred to as solid phase RIA or ELISA. This embodiment may be particularly useful for determining the amount of CD62L, PSGL-1, LFA-1, CD4 and/or CD8 on the surface of cells (cf. also above). On a general basis, in any embodiment of a radiolabel assay or of an enzyme-immunoassay passive adsorption to the solid phase can be used in the first step. Adsorption of other reagents can be prevented by inclusion of wetting agents in all the subsequent washing and incubation steps. It may be advantageous to perform washing to prevent carry-over of reagents from one step to the next.

Various other modifications of ELISA have been used in the art. For example, a system where the second proteinaceous binding molecule or immunoglobulin used in the double antibody sandwich method is from a different species, and this is then reacted with an anti-immunoglobulin enzyme conjugate or an anti-proteinaceous binding molecule enzyme conjugate. This technique comes with the potential advantage that it avoids the labeling of the specific immunoglobulin or proteinaceous binding molecule, which may be in short supply and of low potency. This same technique can be used to assay immunoglobulin or proteinaceous binding molecule where only an impure antigen is available; the specific reactive antigens are selected by the antibody immobilized on the solid phase.

In another example of an ELISA assay for an antigen, a surface, a specific antigen is immobilized on a surface, e.g. a plate used, and the surface is then incubated with a mixture of reference immunoglobulins or proteinaceous binding molecules and a test sample. If there is no antigen in the test sample the reference immunoglobulin or proteinaceous binding molecule becomes fixed to an antigen sensitized surface. If there is antigen in the test solution this combines with the reference immunoglobulin or proteinaceous binding molecule, which cannot then react with the sensitized solid phase. The amount of immunoglobulin/proteinaceous binding molecule attached is then indicated by an enzyme labeled anti-globulin/anti-binding molecule conjugate and enzyme substrate. The amount of inhibition of substrate degradation in the test sample (as compared with the reference system) is proportional to the amount of antigen in the test system.

Yet a further technique that can also be carried out to quantify and thus determine the amount of CD62L, PSGL-1, LFA-1, CD3, CD4 and/or CD8 on the surface of cells in the sample is Fluorescence Microscopy, including Ratio Fluorescence Microscopy. Fluorescence microscopy has long been used as a descriptive adjunct to quantitative biochemical techniques in studies of cellular organization and physiology. In the late 1970s, sensitive imaging detectors became commercially available and gave fluorescence microscopy the potential to be a quantitative tool. However, because of the prohibitive cost and sophistication of high-speed image processing computers, quantitative fluorescence microscopy was generally limited to relatively few laboratories with a specific interest in “digital imaging microscopy.” This situation has changed in the past 10 years with the revolution in digital technology. Inexpensive personal computers are now capable of tasks that once required large mainframe computers.

Integrated optical imaging systems are commercially available that are capable of processing an entire assay from the biological preparation to the final data. In parallel, significant improvements have been made in optical elements and imaging hardware. Sensitive fluorescent indicators of a variety of physiologically important properties have been introduced, and new fluorescent reagents are continually being developed for sensitively and specifically characterizing the intracellular distribution of proteins, nucleotides, ions, and lipids.

As quantitative microscopy becomes more widely available, a user new to fluorescence microscopy should be aware of the factors that may complicate quantification of fluorescence. The amount of fluorescence detected is affected by the properties of illumination sources, the optical and spectroscopic properties of the microscope, and the resolution, sensitivity, and signal-to-noise properties of the detector. Fluorescence emissions are attenuated by the photobleaching that accompanies illumination. At high concentrations of fluorophore, interactions between fluorophore moieties can alter the amount and/or spectrum of fluorescence emissions. For certain fluorophores, fluorescence is also sensitive to the immediate physical environment (i.e., for example, ionic composition) of the fluorophore.

In ratio fluorescence microscopy two fluorescence images are collected and the parameter of interest is quantified as a ratio of the fluorescence in one image to that in the other image. An illustrative example of a ratio fluorescent ion indicator includes, but is not limited to, fluorescein and fura-2, the excitation spectra of which change shape upon binding protons or calcium ions, respectively. In the case of fluorescein, fluorescence excited by 490 run light is efficiently quenched by proton binding, whereas fluorescence excited by 450 nm light is relatively unaffected. Although the quantity of fluorescein fluorescence emitted by a volume when excited with 490 nm light depends on the pH of that volume, it is also affected by other factors, including the concentration of fluorescein in the volume. However, the ratio of fluorescence excited by 90 nm light to that excited by 450 nm depends on pH, but is relatively independent of many variables that affect quantification in single wavelength images: fluorophore concentration, photobleaching, lateral heterogeneity in illumination and detector sensitivity, and differences in optical path length. Spectroscopic variation in illumination and detection is circumvented by calibrating the microscopic system with known pH standards.

Fluorescence ratio images may be collected by sequentially exciting the sample with two different wavelengths of light and sequentially collecting two different images, by exciting the sample with a single wavelength of light and collecting images formed from light of two different emission wavelengths, or by exciting the sample with two wavelengths and collecting emissions of two wavelengths. Ion indicators have been developed for both excitation ratio microscopy (i.e., for example, fura-2 for calcium and fluorescein for pH) and for emission ratio microscopy (i.e., for example, indo-1 for calcium and SNARF for pH).

A further technique suitable for determining the amount of CD62L, PSGL-1, LFA-1, CD3, CD4 and/or CD8 on the surface of cells is fluorescence resonance energy transfer (FRET). In FRET an excited fluorescent donor molecule, rather than emitting light, transfers that energy via a dipole-dipole interaction to an acceptor molecule in close proximity. If the acceptor is fluorescent, then the decrease in donor fluorescence due to FRET is accompanied by an increase in acceptor fluorescence (i.e., for example, sensitized emission). Thus upon excitation of the donor fluorophore, an exciton, which is a radiationless energy emission, is transferred from one fluorophore to the other. As a result, the acceptor fluorophore emits light that is red-shifted in comparison to light that would be emitted from the acceptor fluorophore. The amount of FRET depends strongly on distance, typically decreasing as the sixth power of the distance, so that fluorophores can directly report on phenomena occurring on the scale of a few nanometers, well below the resolution of optical microscopes. Among other purposes, FRET has been used to map distances and study aggregation states, membrane dynamics, or DNA hybridization.

In principle, FRET measurements can provide information about any system the components of which can be manipulated to change the proximity of donors and acceptors on the scale of a few nanometers. In practice, the ability to label a system of interest with appropriate donors and acceptors is constrained by several physical and instrumental factors. In addition to the requirement that donor and acceptor be in close proximity, the donor emission and acceptor absorption spectra should overlap significantly with minimal overlap of the direct excitation spectra of the two fluorophores. Instrumental differences between a fluorescence microscope and a spectrofluorometer, i.e., spatial confinement of the signal, reduced sensitivity, and generally limited wavelength selection, all affect the quality and quantity of information that can be extracted from a FRET experiment using a microscope. The use of FRET in its traditional incarnation as a molecular ruler to measure absolute distances is often not feasible in the fluorescence microscope. Rather, FRET ratio imaging microscopy is often used as an indicator of proximity, subject to some degree of calibration.

The simplest experimental approach is to excite the donor and measure both the direct donor emission “DD” and the sensitized emission “DA” of the acceptor (the first letter represents the species being excited, and the second letter represents the observed emission). The ratio of acceptor-to donor fluorescence, DA/DD, varies between two extremes: no energy transfer and maximal energy transfer. When donor and acceptor are sufficiently distant, no energy transfer occurs and the donor fluorescence (DD) is at its maximum, whereas the sensitized emission is zero. Acceptor fluorescence results only from direct excitation of the acceptor, and DA/DD is at its minimum. The greatest amount of energy transfer occurs when the donor and acceptor are separated by the shortest possible distance, and excited donors lose most of their energy to the acceptor.

Complete quantification of FRET can involve significant calculations, but an estimation of FRET can be obtained easily by measuring the intensity at two fixed time points and taking the ratio of these intensities.

To quantify the relative amount of an acceptor, the acceptor can also be excited directly with the wavelength ideal for acceptor fluorescence, so that “AA” is recorded rather than DA. With AA used as the reference, the ratio DD/AA can also be used as a measure of FRET. Measurement of AA does not generally affect the measurement of DD because acceptor excitation wavelengths are always longer (lower energy) than donor excitation wavelengths, thus avoiding photobleaching of the donor.

Although photobleaching should usually be minimized, it can in some cases actually be exploited to measure FRET. Photobleaching of the donor usually occurs when it is in the excited state: before fluorescence emission occurs there is some probability that photobleaching will remove that fluorophore from the excited state, and also from future excitation emission cycles. When FRET occurs, the donor is removed from the excited state before emission or photobleaching, and the bleach rate decreases because that donor remains available for another cycle of excitation emission. The efficiency of FRET can be determined from the bleach rate of donor fluorescence in the presence of acceptor compared with the bleach rate of the donor in the absence of acceptor. Experimentally, the instantaneous intensity, I(t), is normalized to the initial intensity I(0) and the decay of fluorescence intensity is analyzed. A major advantage of the photobleaching method is that it uses only a single excitation wavelength and only a single emission wavelength. The bleach rate of the donor in the absence of acceptor should be measured under experimental conditions identical to those for the donor-acceptor pair, because bleaching rates can vary significantly for different intracellular environments.

If (i) the amount of FRET is relatively small; (ii) the acceptor is not fluorescent; or (iii) rapid photobleaching prevents measurement of static fluorescence intensities, a photobleaching method may provide the only practical measurement of FRET. In particular, the photobleaching method should be useful with the high illumination intensities typical with lasers used for confocal microscopy.

### Quantification and Comparability of Biomarker Levels

Determining the level or amount of CD62L, PSGL-1, LFA-1, CD4, CD8 and/or CD3 in the sample typically involves the formation of signals, e.g. signals generated by a detectable marker (supra) that can be quantified. Quantifying the signals in order to determine the level of e.g. CD62L, PSGL-1, CD3 and/or LFA-1 in the sample may be carried out by comparing obtained signals with those of one or more reference measurements. As will be apparent from the above, the word “comparing” as used herein refers to a comparison of parameters or values in terms of absolute amounts/levels that correspond to each other. As an example, a number of cells is compared to a reference number of cells, a concentration is compared to a reference concentration, or a signal intensity obtained from a test sample is compared to the intensity of a corresponding type of signal obtained in a reference sample. A respective reference measurement may be based on the signal generated by a known amount of CD62L, PSGL-1, LFA-1 and/or CD3. Such a known amount of CD62L, PSGL-1, LFA-1 and/or CD3 may for example be present in a sample with a composition that resembles the sample from the subject, in which the amount of CD62L, PSGL-1, LFA-1 and/or CD3 is to be determined. A respective reference sample may be taken to define an external reference sample. In some embodiments of a method of the invention an internal reference sample may in addition or alternatively be used. Such an internal reference sample is a sample obtained from the subject at a previous point of time. The amount of CD62L, PSGL-1, LFA-1 and/or CD3 in such a sample may be determined to identify the changes in CD62L, PSGL-1, LFA-1 and/or CD3 levels in the subject. In some embodiments the level or amount of CD62L, PSGL-1, LFA-1 and CD3, respectively, in the sample may be normalized by a comparison to the level of one or more other proteins, typically cell surface proteins that are known in the art to be stably expressed. In some embodiments a technique of determining the number, amount or ratio of T cells that have e.g. CD62L, PSGL-1 and/or LFA-1 on their surface includes calibrating the analysis equipment. In embodiments where flow cytometry is used, a standardized blood cell sample may for example be used such as the IMMUNO-TROL® Control Cells commercially available from Beckman Coulter Inc. (Fullerton, Calif., USA, order No. 6607077).

In some embodiments of a method or use of the invention the amount or level of T cells that have both CD62L and CD3 determined in the sample may be compared to a threshold value. In some embodiments of the method of the invention the amount or level of T cells that have both LFA-1 and CD3 determined in the sample may be compared to a threshold value. In some embodiments the amount/level of T cells that have both PSGL-1 and CD3 determined in the sample may be compared to a threshold value. In some embodiments the amount of T cells that have PSGL-1, CD62L and CD3 determined in the sample may be compared to a threshold value PSGL-1. In some embodiments the amount of T cells that have PSGL-1, CD62L, LFA-1 and CD3 determined in the sample may be compared to a threshold value PSGL-1. In some embodiments the amount or level of T cells that have CD62L, LFA-1 and CD3 determined in the sample may be compared to a threshold value. In some embodiments the ratio of T cells that have CD62L and/or LFA-1 and CD3 to T cells that have only CD3, but not CD62L and/or LFA-1, may be determined in the sample may and compared to a threshold ratio. In some embodiments the ratio of T cells that have both CD62L and CD3 or both LFA-1 and CD3 to all T cells that have CD3 may be determined in the sample may and compared to a threshold ratio. In some embodiments the ratio of T cells that have CD62L, LFA-1 and CD3 determined in the sample may be compared to a threshold value. In some embodiments the amount or level of T cells that have CD62L and/or PSGL-1, as well as CD3 determined in the sample may be compared to a threshold value. In some embodiments the ratio of T cells that have CD62L, PSGL-1 and/or LFA-1 and CD3 to T cells that have only CD3, but not CD62L, PSGL-1 and/or LFA-1, may be determined in the sample and may compared to a threshold ratio. In some embodiments the ratio of T cells that have both PSGL-1 and CD3 to all T cells that have CD3 may be determined in the sample may and compared to a threshold ratio. In some embodiments the ratio of T cells that have CD62L, PSGL-1, LFA-1 and CD3 determined in the sample may be compared to a threshold value.

A respective threshold value may in some embodiments be a predetermined threshold value. In some embodiments the threshold value is based on the amount of cells having both CD62L and CD3 in a control sample or both LFA-1 and CD3 in a control sample. Likewise, such a threshold value is based on the amount of cells having both PSGL-1 and CD3 in a control sample or both PSGL-1 and CD3 in a control sample. As applicable, in some embodiments the threshold value is based on the amount of cells having CD62L, PSGL-1, LFA-1 and CD3 in a control sample. In some embodiments the threshold value is a threshold ratio based on the ratio of cells that have both CD62L and/or LFA-1 and CD3 to T cells that have only CD3, but not CD62L and/or not LFA-1, or to all T cells that have CD3 in a control sample. In some embodiments the threshold value is a threshold ratio based on the ratio of cells that have both PSGL-1 and CD3 to T cells that have only CD3, but not PSGL-1, or to all T cells that have CD3 in a control sample. A respective control sample may have any condition that varies from the sample main measurement itself. Such a control sample may be a sample of include or essentially consist of the corresponding body fluid as the sample from the subject. A control sample may for example be a sample, such as a blood sample, a plasma sample, a serum sample or a cerebrospinal fluid (liquor) sample, of a subject known not to suffer from PML or from aspects of a JCV induced disease. In some embodiments a respective control sample is from a subject that is age-matched. In some embodiments a respective control sample is from a subject that is known not to have a confounding disease, in some embodiments from a subject known not to have either HIV/AIDS or PML, or from a subject known to suffer from MS, as applicable, and in some embodiments from a subject known not to have a disease. As can be taken from FIG. 1D and FIG. 3A, both HIV infection and treatment with Natalizumab are generally associated with a reduced expression of CD62L on T cells, without occurrence of PML having taken place. Hence, it may in some embodiments be desirable to select a control sample as originating from a subject known not to suffer from PML, but to be under therapy with an [symbol] ₄-integrin/VLA-4 blocking agent such as Natalizumab or suffering from HIV infection, as applicable. As can further be taken from FIG. 3B, both HIV infection and treatment with Natalizumab may in some cases be associated with a, possibly slightly, increased expression of PSGL-1 on T cells, without occurrence of PML having taken place. In some embodiments it may therefore be desirable to select a control sample as originating from a subject known not to suffer from PML, but to be under therapy with an [symbol] ₄-integrin/VLA-4 blocking agent such as Natalizumab or suffering from HIV infection, as applicable.

In some embodiments a threshold value is based on a control or reference value obtained concomitantly with the value of the sample from the subject. In some embodiments a respective control or reference value is determined at a different point in time, for example at a point in time earlier than the measurement of the sample from the subject is carried out. It is understood that the terms control and reference may in some embodiments be a range of values.

Population studies may also be used to select a threshold value. Receiver Operating Characteristic (“ROC”) arose from the field of signal detection theory developed during World War II for the analysis of radar images, and ROC analysis is often used to select a threshold able to best distinguish a diseased subpopulation from a nondiseased subpopulation. A false positive in this case occurs when a person tests positive, but actually does not have the disease. A false negative, on the other hand, occurs when the person tests negative, suggesting the person is healthy, when it actually does have the disease. To draw a ROC curve, the true positive rate (TPR) and false positive rate (FPR) are determined as the decision threshold is varied continuously. Since TPR is equivalent with sensitivity and FPR is equal to 1—specificity, the ROC graph is sometimes called the sensitivity vs (1-specificity) plot. A perfect test will have an area under the ROC curve of 1.0; a random test will have an area of 0.5. A threshold is selected to provide an acceptable level of specificity and sensitivity.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

The comparison to a threshold value, which may be a predetermined threshold value, can be carried out manually, semi-automatically or in a fully automated manner. In some embodiments the comparison may be computer assisted. A computer assisted comparison may employ values stored in a database as a reference for comparing an obtained value or a determined amount, for example via a computer implemented algorithm. Likewise, the comparison to a reference measurement may be carried out manually, semi-automatically or in a fully automated manner, including in a computer assisted manner. A computer assisted comparison may rely on the storage of data, for instance in connection with determining a threshold value, on the use of computer readable media. Suitable computer readable media may include volatile, e.g. RAM, and/or non-volatile, e.g. ROM and/or disk, memory, carrier waves and transmission media such as copper wire, coaxial cable, fibre optic media. Exemplary carrier waves may take the form of electrical, electromagnetic or optical signals conveying digital data streams along a local network or a publically accessible network such as the Internet.

The level of expression of CD62L, PSGL-1 and/or LFA-1 determined in or from a sample of a subject may be expressed in terms of cell numbers, i.e. the number of T cells that are positive for CD62L, for PSGL-1 and/or for LFA-1. The level of expression of CD62L, PSGL-1 and/or LFA-1 may also be expressed in terms of the total amount of CD62L, PSGL-1 and/or LFA-1 in a sample. As explained above, where immobilization of cells onto a surface is employed, for example an immobilized binding partner specific for T cells, the total amount of CD62L, PSGL-1 and/or LFA-1 present on the respective cells may be used to express the total amount of CD62L, PSGL-1 and/or LFA-1. In some embodiments a high level of soluble CD62L can be expected to be included in the sample from a patient. Soluble CD62L, i.e. CD62L that is not immobilized on a cell surface, originates for example from granulocytes. In such embodiments it may be advantageous to distinguish soluble CD62L and CD62L present on the surface of cells or to remove soluble CD62L before detecting CD62L in the detection method. Whether high levels of soluble CD62L are to be expected in a sample can easily be tested by for instance measuring a single value of the sample with and/without immobilizing T cells and subsequently washing the same. A significant difference of the obtained values indicates a high amount of soluble CD62L in the sample. The term “significant” is used to indicate that the level of decrease or increase is of statistical relevance, and typically means a deviation of a value relative to another value of about 2 fold or more, including 3 fold or more, such as at least about 5 to about 10 fold or even more.

The expression level of CD62L, PSGL-1 and/or LFA-1 determined in or from a sample of a subject can be compared to a single control sample or a plurality of control samples, such as a sample from a control subject, in any suitable manner. As an illustrative example, the expression level of CD62L, PSGL-1 and/or LFA-1 in a control sample can be characterized by an average (mean) value coupled with a standard deviation value, for example at a given time point. In some embodiments the expression level of CD62L, PSGL-1 and/or LFA-1 in a subject may be considered different when it is one standard deviation or more higher or lower than the average value of the corresponding expression level determined in one or more control samples. In some embodiments the determined expression level of CD62L, PSGL-1 and/or LFA-1 is regarded as different where the obtained value is about 1.5 standard deviations higher or lower, including about two, about three, about four or more standard deviations higher or lower than the average value determined in a control sample. In some embodiments the determined expression level of CD62L, PSGL-1 and/or LFA-1 is regarded as different where the obtained value is about 1.2 times or more higher, or lower, including about 1.5 times, about two fold, about 2.5-fold, about three fold, about 3.5 fold, about 4-fold, about 5-fold or more higher or lower than the expression level determined in a control sample. In some embodiments the determined expression level of CD62L, PSGL-1 and/or LFA-1 is regarded as different where the obtained value is about 0.8-fold or less than the expression level determined in a control sample. The determined expression level of CD62L, PSGL-1 and/or LFA-1 may for example be regarded as different if a value is about 70%, such as about 60% or about 50% lower than the expression level determined in a control sample. In some embodiments an expression level of CD62L, PSGL-1 and/or LFA-1 is regarded as different if the obtained value is about 40%, including about 30% lower than the expression level determined in a control sample. An expression level of CD62L, PSGL-1 and/or LFA-1 is in some embodiments regarded as different if the obtained value is about 25%, such as about 20% or lower than the expression level determined in a control sample.

A predetermined threshold value may in some embodiments be set on the basis of data collected from one or more subjects known not to suffer from and not to be at elevated risk of PML or of aspects of a JCV induced disease. In some embodiments a certain percentile of such data may be used as a threshold value. The range of the values of a set of data obtained from such individuals can be divided into 100 equal parts, i.e. percentages of the range can be determined. A percentile represents the value within the respective range below which a certain percent of the data fall, in other words the percentage of the values that are smaller than that value. For example the 95th percentile is the value below which 95 percent of the data are found. In some embodiments a level of CD62L, PSGL-1 and/or LFA-1 may be regarded as decreased or low if it is below the 90^{th} percentile, below the 80^{th} percentile, below the 70^{th} percentile, below the 60^{th} percentile, below the 50^{th} percentile or below the 40^{th} percentile.

In evaluating the risk of occurrence of a JCV induced disease such as PML, in some embodiments a reduced amount of CD62L, PSGL-1 and/or LFA-1 relative to a threshold value, indicates an elevated risk of occurrence of a JCV induced disease, typically PML, in a subject. An amount of CD62L, PSGL-1 and/or LFA-1 that is not below a threshold value or that is above a threshold value indicates that there is no elevated risk of occurrence of PML in the subject. A level of CD62L, PSGL-1 and/or LFA-1 below a threshold value may indicate a condition where the subject is in need of therapy or in need of a change of a therapy to which the subject is being exposed. If a level of CD62L, PSGL-1 and/or LFA-1 is detected that is above a, possibly predetermined, threshold value, this may indicate that no PML has occurred, as well as that the risk of occurrence of PML is low. Likewise, a level of CD62L, PSGL-1 and/or LFA-1 that is about the same as a threshold value may indicate that no PML has occurred, as well as that the risk of occurrence of PML is not elevated when compared to other subjects in a similar disease state. The risk that the subject may suffer from PML may be low.

### Repeated Measurements and Monitoring of Biomarker Levels

In some embodiments a plurality of measurements is carried out on a plurality of samples from the same patient. In each of the samples the level of expression of CD62L, PSGL-1 and/or LFA-1 is determined. Typically the level of expression determined in each of the samples is compared to a threshold value as detailed above. In some embodiments the plurality of samples from the same individual is taken over a period of time at certain time intervals, including at predetermined time intervals. Such an embodiment may be taken as a method of monitoring the expression of CD62L, PSGL-1 and/or LFA-1. Matching samples may in some embodiments be used to determine a threshold value for each corresponding time point. The average value may be determined and the standard deviation calculated for each given time point. A value determined in the sample from the subject falling outside of the mean plus 1 standard deviation may be indicative of an increased risk of occurrence of a JCV induced disease such as PML.

In some embodiments, a method of the invention includes monitoring the risk of occurrence of a JCV induced disease such as PML of a subject suffering from HIV or from an autoimmune disease, and under treatment with an α₄-integrin blocking agent, LPAM-1 blocking agent and/or a VLA-4 blocking agent, or with an anti-retroviral therapy, as applicable. In the method expression levels of CD62L, PSGL-1 and/or LFA-1 are determined and the result(s) is/are correlated to the likelihood of occurrence or nonoccurrence of a JCV induced disease, typically PML, in the subject. As explained above, the measured concentration(s) may be compared to a threshold value. When the measured expression level is below the threshold, an enhanced risk of PML may be assigned to the subject; alternatively, when the measured concentration is at or above the threshold, no elevated risk of PML may be assigned to the subject.

In one embodiment the level of CD62L, PSGL-1 and/or LFA-1 is measured at certain, e.g. predetermined, time intervals. Samples from the subject may be provided that have been obtained at the corresponding time points. As an illustrative example, samples may be taken from the same subject after a time interval of about 3 months, including about every month. In some embodiments samples may be taken from the same subject at a time interval of about 6 months. In some embodiments a sample may be taken from the same subject after a time interval of about a year, i.e. about 12 months. In some embodiments a sample may be taken from the same subject after about 18 months. A value obtained from a respective sample may in some embodiments be compared to a sample taken from the same subject at a previous point of time, for example the previous measurement and/or the first measurement taken. In this way a change in the level of CD62L, PSGL-1 and/or LFA-1 may be detected. Matching samples may in some embodiments be used to determine a threshold value for each corresponding time point. The average value may be determined and the standard deviation calculated for each given time point. As an illustrative example, a value determined in the sample from the subject falling outside of the mean plus 1 standard deviation may for instance be indicative of the occurrence or of the risk of occurrence of PML.

In some embodiments a measurement carried out at a certain time point is repeated if during monitoring, i.e. measuring the amount of CD62L, PSGL-1 and/or LFA-1 at certain time intervals a decrease is detected, in particular if a decrease beyond a threshold value is detected. In some embodiments time intervals after which the level of CD62L, PSGL-1 and/or LFA-1 are being determined may be shortened if during monitoring of the amount of CD62L, PSGL-1 and/or LFA-1 a decrease has been detected. As an illustrative example, a decrease in levels of one or more of CD62L, PSGL-1 and/or LFA-1 may have been found at a certain point of time during measurements carried out at intervals of 12 months or during measurements carried out at intervals of 18 months. After such a decrease in levels has been found, monitoring of the level of CD62L, PSGL-1 and/or LFA-1 may be continued at time intervals of about a month. As indicated above, monitoring the amount of CD62L, PSGL-1 and/or LFA-1 may be included in the context of monitoring a therapy, for example in order to assess the efficacy thereof or to evaluate a subject's response to a certain treatment.

In embodiments where the subject is to be treated, for example with an α₄-integrin blocking agent, a LPAM-1 blocking agent and/or a VLA-4 blocking agent, monitoring expression levels may in some embodiments start prior to the treatment. In some embodiments monitoring may start at the same time or at an early stage of the treatment, e.g. administration of an α₄-integrin blocking agent, a LPAM-1 blocking agent and/or a VLA-4 blocking agent.

As indicated above, in some embodiments a method or use according to the present invention includes measuring CD62L, PSGL-1 and/or LEA-1 expression on T cells in a sample or obtained from a sample, and comparing the result obtained therefrom to a reference value. In the context of a therapy or of HIV infection, in some embodiments detecting the level of CD62L expressing T cells as well as monitoring the same includes determining whether one or more of the following indications is present:

(1) In the context of therapy with an α₄-integrin blocking agent and/or a VLA-4 blocking agent, a lack of CD62L expression may be observed after administration of a α₄-integrin/VLA-4 blocking agent. The lack of CD62L expression may be observed at a point of time, such as within about the first week. In some embodiments lack of CD62L expression may be detected within about the second week or within about the third week. A lack of CD62L expression may in some embodiments be detected within about the 1^{st} month, within about the 2^{nd} month, within about the 3^{rd} month, within about the 4^{th}, 5^{th}, 6^{th}, 7^{th}, 8^{th}, 9^{th}, 10^{th}, 11^{th}, 12^{th}, 13^{th}, 14^{th}, 15^{th} or within about the 16^{th} month. In some embodiments a lack of CD62L expression may be detected in the 17^{th} month. In some embodiments lack of CD62L expression may be detected in the 18^{th} month. A lack of CD62L expression may in some embodiments be detected within the 19^{th}, the 20^{th}, 21^{st}, 22^{nd}, 23^{rd}, 24^{th}, 25^{th} month or longer. For example, FIG. 12 shows that only very low levels CD62L in a sample from one subject who later developed PML could be detected after 15 months of treatment.

(2) A differential expression level of CD62L on the T cell surface compared to a reference level obtained from “control subjects”, as indicated above. A differential expression in some embodiments refers to a “decreased” expression compared to a reference. In the context of therapy with an α₄-integrin/VLA-4 blocking agent control subjects may be defined as those who underwent α₄-integrin/VLA-4 blocking agent treatment for about one year or more, such as about 1.5 years or more, about 2 years or more, or about 3 years or longer but who have not been diagnosed with PML. The samples to be compared are in some embodiments obtained from the same or substantially the same time point after the initiation of the treatment. For example, a 1-month sample is in some embodiments compared to another 1-month sample. In the context of HIV infection a control subject may be an individual of a comparable stage of AIDS, who is known not to have PML. A “differential” expression is observed by comparing a measured expression level to a corresponding level of one or more control subjects. In case of a reduced level of a biomarker, as in the context of a decreased level of CD62L, the differential expression is a “decreased” expression compared to a reference.

The expression of CD62L in/on T cells determined from a sample of a subject can be compared to one or more control subjects in any suitable manner. For example, the expression of CD62L in the control subject can be characterized by an average (mean) value coupled with a standard deviation value at a given time point. The expression of CD62L in a subject may for instance be considered different when it is more than one standard deviation different from the average value.

(3) A low number of T cells expressing CD62L. This can be represented by, for example, ratio of such cells to total PBMC, number of cells per sample (e.g. mm³ blood), ratio of such cells to all T cells, or otherwise, as suitable for such representation. When the number is represented by percentage of T cells expressing CD62L, a “low” percentage is defined as less than about 10%. In some embodiments a low percentage is defined as less than about 9%, such as less than about 8%, such as less than about 7%, 6%, 5%, 4%, or 3%. A low percentage of T cells expressing CD62L is in some embodiments defined as less than about 2%. In some embodiments a low percentage is defined as 1% or less, including about 0.5%, or less. If other methods are employed, a skilled person is able to convert the values here given according to the method used and common knowledge. If the value observed is repeatedly low, for instance persistently low over a period of a plurality of months, such as about 5 months or more, the subject is more likely to suffer from PML at present or in the future. In some embodiments an extended period of time is a period of 6 months or more, such as about 7, 8, 9, 10, or 11 months. In some embodiments an extended period of time is a period of 12 months or longer. As an illustrative example, FIG. 12 shows that the CD62L levels on T cells from subjects who later developed PML were persistently low.

(4) A lack of “recovery” of the percentage of T cells which expresses CD62L. The term “recovery” is determined by comparing the obtained amount or level to a threshold value, which may be based on a reference level. As used herein, “recovery” is defined as a return of the percentage of T cells which express CD62L back to the range of the reference level or higher.

The reference level for this purpose can be determined by various methods. In some embodiments, the reference is obtained from the same subject at the first month of the treatment of α₄-integrin/VLA-4 blocking agent. In some embodiments, the reference level may be a determined value from an earlier point in time, such as about 3 months ago. In some embodiments the earlier point in time may be 4 months ago, such as about 5 months ago. The earlier point in time may in some embodiments be about 6 months ago. In some embodiments the earlier point in time may be about 7 or about 8 months ago, or historical reference level from past course of treatment. In some embodiments, the reference level is obtained from one or more control subjects, such as about 30 or more control subjects who underwent treatment of α₄-integrin/VLA-4 blocking agent for more than 1 year. The reference level is in some embodiments obtained from about 40 or more control subjects, including about 50 or more, about 60 or more or about 70 or more control subjects who underwent treatment with an α₄-integrin/VLA-4 blocking agent for more than 1 year, such as more than about 1.5 years, more than about 2 years, about 2.5 years, about 3 years, or more. In some embodiments, the reference level is measured within about the first month after the first administration of the α₄-integrin/VLA-4 blocking agent.

FIG. 12 shows that the CD62L levels of control subjects recovered (exceeding the reference level taken at the first month) after 15 months of α₄-integrin/VLA-4 blocking agent treatment.

In some embodiments, a sample from the subject to be tested is taken about one month after the treatment. PMBC is isolated from the sample and subjected to a suitable detection technique such as FACS analysis. The percentage of T cells, including CD4⁺ T cells and/or CD8⁺ T cells, which are CD62L positive is measured and compared to a reference level derived from one or more control subjects. If the measured value is lower or higher than the threshold value, it is indicative of an increased risk to develop PML.

For instance, the following indicate reference levels for CD62L that can be used to set a threshold value:

TABLE 1
Exemplary reference values for CD62L
for individuals receiving Natalizumab

| | % of CD4⁺ T cells | | reference level (mean % of |
|---|---|---|---|
| | positive for CD62L | | CD4⁺CD62L⁺ T cells |
| Month | (mean (standard deviation)) | | minus 1 standard deviation) |
| 0 (before | 53.7 | (10.2) | 43.5 |
| treatment) | | | |
| 1 | 28.2 | (7.2) | 21.0 |
| 3 | 36.9 | (17.8) | 19.1 |
| 6 | 20.0 | (14.9) | 5.1 |
| 12 | 16.7 | (16.2) | 0.5 |
| 15-20 | 40.0 | (13.9) | 26.1 |
| 21-25 | 41.8 | (10.3) | 31.5 |
| 26-30 | 44.9 | (12.5) | 32.4 |
| 31-35 | 34.0 | (6.9) | 27.1 |
| 36-40 | 38.0 | (18.1) | 19.9 |
| 41-45 | 33.5 | (6.4) | 27.1 |
| 46-50 | 32.3 | (2.3) | 30.0 |
| 51-55 | 39.5 | (20.7) | 18.8 |

As an illustrative example, the reference level for a subject having received 1 month treatment of Natalizumab may be 21%. An expression level that is lower than 21% may be considered “different” and indicate a risk for PML.

When one of the above indications is observed, for example, when there is a lack of CD62L expression, or when low expression of CD62L persists for an extended period of time, the physician should consider, combined with other information available, measures such as stopping or temporarily withholding the treatment, adjusting the dosage, or performing plasma exchange, until the expression level increases or recovers. It may be possible to resume the treatment after the expression level of the biomarkers in the present invention has recovered or increased.

As can be taken from FIG. 3A and FIG. 12, levels of CD62L on T cells tend, with the exception of about the initial 12 months of treatment at all, to remain within a relatively stable range during treatment of relapsing remitting multiple sclerosis with an α₄-integrin/VLA-4 blocking agent. In contrast thereto, in the course of HIV/AIDS levels of CD62L on T cells tend to, possibly slightly, decrease. Nevertheless a drop of CD62L on T cells can typically be observed after onset of PML in either situation, i.e. whether a subject has HIV/AIDS or is under α₄-integrin/VLA-4 blocking agent treatment (cf. also FIG. 12). It is thus in some embodiments helpful to monitor the time course of CD62L levels on T cells of an individual, whether HIV positive, under α₄-integrin/VLA-4 blocking agent therapy or any other particular condition. In this way any unexpected alteration of CD62L levels can be detected. Such alteration is an indication of an elevated risk of PML.

As explained above, in some embodiments of a method or use of the invention the expression level of LFA-1 in the sample is determined. In some embodiments the expression level of LFA-1 is determined at a plurality of time points, for example by determining the expression level of LFA-1 in a plurality of samples, which have been obtained from the same subject at particular time points over a period of time. If the expression level of LFA-1 observed is persistently different from a threshold value over an extended period of time, the subject is at a higher risk to suffer from PML. As an example in this regard, FIG. 11 shows that the LFA-1 levels of two patients who later developed PML were persistently lower than that from control patients after month 6 and 12.

For instance, a reference level of LFA-1 as indicated in the following can be used to seta threshold value:

TABLE 2
Exemplary reference values for LFA-1
for individuals receiving Natalizumab

| | % of CD4⁺ T cells | | reference level (mean % of |
|---|---|---|---|
| | positive for LFA-1 | | CD4⁺LFA-1⁺ T cells |
| Month | (mean (standard deviation)) | | minus 1 standard deviation) |
| 0 (before | 35.5 | (13.6) | 21.9 |
| treatment) | | | |
| 1 | 31.2 | (12.7) | 18.5 |
| 3 | 25.4 | (7.6) | 17.8 |
| 6 | 23.8 | (9.8) | 14.0 |
| 12 | 28.9 | (10.2) | 18.7 |
| 15-20 | 47.3 | (16.3) | 31.0 |
| 21-25 | 59.9 | (7.5) | 52.4 |
| 26-30 | 50.4 | (16.8) | 33.6 |
| 31-35 | 26.0 | (19.8) | 6.2 |
| 36-40 | 40.6 | (18.9) | 21.7 |
| 41-45 | 37.0 | (5.7) | 31.3 |
| 46-50 | 34.5 | (11.8) | 22.7 |
| 51-55 | 42.2 | (17.2) | 25.0 |

For the purpose of the present invention, the detection of LFA-1 expression can also include detecting the protein or mRNA of CD11a and Runx3. In this case, the determining the risk may be carried out using generally the same approach as for the LFA-1 protein.

As should be apparent from the above, if for example a level of T cells, which have CD62L and/or both CD62L and LFA-1 is detected that is below a (e.g. predetermined) threshold value, this may indicate a risk that the subject will have PML, often at a later point of time. In embodiments where the sample is from an HIV positive subject, if a level of T cells that have both CD62L and LFA-1 is detected that is below a predetermined threshold value, this may indicate the need to change therapy. As explained above, administration of an α₄-integrin blocking agent, a LPAM-1 blocking agent and/or a VLA-4 blocking agent may be discontinued, including interrupted, and to the subject an agent such as a compound and/or an antibody may be administered that is known or suspected to be effective against JCV. A level of T cells that have both CD62L and LFA-1 below a predetermined threshold value may also indicate a condition where the subject is suffering from PML. In case it is suspected that a subject is suffering from PML the practitioner will usually carry out MRI imaging. It may for example be analysed whether lesions in subcortical white matter exist. The presenting PML symptoms most commonly include changes in cognition, behaviour, and personality, but in some cases seizures may be the first clinical event. Such symptoms may occur either alone or, associated with motor, language, or visual symptoms.

The above said applies to the detection of PSGL-1 mutatis mutandis. If a level of T cells that have PSGL-1 and/or both PSGL-1 and CD62L is detected that is below a (e.g. predetermined) threshold value, this may indicate a risk that the subject will have PML, often at a later point of time. Typically, a level of PSGL-1 on T cells that is below a threshold value is indicative of a risk of PML. FIG. 3B shows that PSGL-1 levels on T cells tend to increase slightly during treatment with an α₄-integrin blocking agent, a LPAM-1 blocking agent and/or a VLA-4 blocking agent. However, before onset of PML during treatment with a VLA-blocking agent, levels of PSGL-1 on T cells typically drop. As can likewise be taken from FIG. 3B, PSGL-1 levels on T cells of HIV infected subjects typically drop after onset of PML.

If the expression level of either PSGL-1 or CD62L on T cells from a subject is detected that is below a threshold value, this may also indicate a risk that the subject will have PML. If the expression level of PSGL-1 observed is for instance persistently different from, in particular below, a threshold value over an extended period of time, the subject is diagnosed to be at an elevated risk to develop PML. If the expression level of both PSGL-1 and CD62L observed is persistently different from, in particular below, a threshold value over an extended period of time, the subject is also diagnosed to be at an elevated risk to develop PML. Likewise, if the expression level of either PSGL-1 or CD62L observed is persistently lower than a threshold value over an extended period of time, the subject is also diagnosed to be at an elevated risk to develop PML. Where the sample is from an HIV positive subject, if a level of T cells that have PSGL-1 is detected that is below a threshold value, this may indicate the need to change therapy. A level of T cells that have PSGL-1 below a threshold value may also indicate a condition where the subject is suffering from PML. A level of T cells that have at least one of PSGL-1 and CD62L below a threshold value may also indicate a condition where the subject is suffering from PML.

As explained above, PML is a risk factor that can be taken as an adverse effect in treating a subject with an α₄-integrin blocking agent, a LPAM-1 blocking agent and/or a VLA-4 blocking agent. In contrast thereto, PML is an inherent risk associated with HIV infection. The introduction of highly active antiretroviral therapy (HAART) has improved both the clinical and radiologic findings in HIV-infected subjects and reduced the number of opportunistic infections. In countries that use HAART, AIDS (acquired immunodeficiency syndrome) dementia complex is becoming the most common neurologic complication of HIV infection, whereas opportunistic infections are still the major cause of neurologic complications in patients from countries that do not commonly use HAART. Immune reconstitution inflammatory syndrome, which occurs in some patients in the weeks to months after the institution of HAART, may alter the typical imaging appearance of infectious diseases involving the CNS. The advent of HAART, which has been used in Western countries to treat HIV-infected patients since 1996, has resulted in a decline in the incidence of neurologic complications, especially those caused by opportunistic infections. In countries where HAART is available, cognitive dysfunction and peripheral neuropathies that are caused directly by HIV represent the majority of cases of HIV-related neurologic disorders; in other countries, opportunistic infections of the CNS are more common.

In embodiments where the subject is HIV positive, determining the risk of occurrence of PML in a subject does therefore not necessarily command the suspension of the therapy currently used. In this regard data indicate that HAART should rather be started if the risk of occurrence of PML is determined, if an HIV positive subject is not yet under HAART, as already detailed above. Nevertheless, where an increased risk of PML is determined for a particular subject, the combination of anti-retroviral compounds, typically including one or more reverse transcriptase inhibitors and optionally a protease inhibitor, used in HAART may be discontinued. In such an embodiment discontinuing the administration of the combination of anti-retroviral compounds may include a substitution therapy. In such a substitution therapy an alternative combination of anti-retroviral compounds may be administered to the subject.

Where the subject is an HIV positive subject, if an increased risk of PML is determined, a therapy may be initiated that aims at prolonging the time until PML occurrence, thereby giving the organism more time to develop a JCV immune response. In this regard an HT₂ₐ antagonist may be administered, as already indicated above. Where an increased risk of PML has been determined for an HIV positive subject, a therapy may also be initiated that aims at reducing the JCV load in the subject's organism.

On a general basis the finding that viral protein 1 (VP1) of JCV attaches to the oligosaccharide lactoseries tetrasaccharide c (LSTc) on host cells and the determination of the crystal structure of VP1 in complex with LSTc (Neu et al., 2010, supra) can be expected to allow the development of pharmaceutically active compounds in the foreseeable future that are effective in the treatment of JCV infection, including PML. It has been found that glycans terminating in the LSTc motif serve as main receptors for JCV and that JCV infection can be blocked specifically by incubation with soluble LSTc. In addition, the termini of long oligosaccharide chains of the so called ‘I’ antigen, which is expressed on a high proportion of human peripheral lymphocytes, can be taken to define homologs of LSTc, in which a GlcNAc replaces the terminal Glc of LSTc (ibid.).

In some embodiments of a method according to the invention prior to a planned treatment the level of CD62L on T cells from a subject is determined as detailed above. If a decreased level of CD62L present on T cells, relative to a threshold value, is determined, an increased risk of PML occurrence may be diagnosed. In embodiments where the subject is HIV positive the planned therapy may be adjusted in order to achieve a particularly fast and effective immune restoration and/or in order to assist the subject's organism to provide JCV specific T cell responses. In some embodiments it may be considered to include a HT₂ₐ antagonist into a planned therapy. As indicated above, in some embodiments the level of CD62L on T cells from a subject may be monitored over time. For this purpose frozen samples that were obtained from the subject at different time points may for instance be analysed within the same measurement. The level of CD62L on T cells may for instance be measured at time intervals of one or more months such as about every 6 months, about every 8 months, about every 10 months, about every 12 months or about every 14 months during a treatment, for instance with an α₄-integrin/VLA-4 blocking agent, or as long as the subject is diagnosed to suffer from a disease such as HIV or multiple sclerosis. A decrease in the level of CD62L on T cells may indicate that the subject is at a risk of developing PML. Depending on further diagnosis results, a change of the level of CD62L on T cells may also indicate that the subject is developing PML. In some embodiments where the subject is undergoing treatment with an α₄-integrin blocking agent, a LPAM-1 blocking agent and/or a VLA-4 blocking agent the level of CD62L on T cells may be determined before a treatment with a respective blocking agent is begun. Thereafter a further analysis of the level of CD62L on T cells may for instance be carried out about 1.5 years after the start of treatment. Subsequently the level of CD62L on T cells from the subject may be analysed about every 6 months.

In some embodiments of a method according to the invention prior to a planned treatment the level of PSGL-1 on T cells from a subject is determined (supra). A respective measurement of the level of PSGL-1 may for instance serve as a reference for later measurements that may be carried out during the course of the planned therapy. The level of PSGL-1 on T cells may for instance be measured at time intervals of one or more months such as about every 3 months, about every 6 months, about every 8 months, about every 10 months, about every 12 months or about every 14 months during a treatment, for instance with an α₄-integrin blocking agent, a LPAM-1 blocking agent and/or a VLA-4 blocking agent, or as long as the subject is diagnosed to suffer from a disease such as HIV or multiple sclerosis. The detection of decreased level of PSGL-1 on T cells, relative to a threshold value, is determined, may be the basis of or a factor leading to the prediction/diagnosis of an increased risk of PML occurrence. Levels of PSGL-1 may also be compared between measurements carried out a different time points or between samples taken at different time points from the subject. A decrease of the level of PSGL-1 on T cells may indicate an increased risk of PML occurrence. Again, in case the subject is HIV positive the planned therapy may be adjusted in order to achieve a particularly fast and effective immune restoration and/or in order to assist the subject's organism to provide JCV specific T cell responses. In some embodiments administration of a HT₂ₐ antagonist may be considered (supra).

Several methods according to the present invention can be used to predict whether a subject is likely to develop PML. This is of particular importance since no PML therapy is currently available and overall mortality is above 50%, as explained above. In addition, once PML is diagnosed in a subject undergoing treatment with an α₄-integrin blocking agent, a LPAM-1 blocking agent and/or a VLA-4 blocking agent, plasma exchange or immunoadsorption is required in order to more rapidly remove the respective blocking agent from plasma and to speed up the reconstitution of immune surveillance. In this regard immunoadsorption is only established as a medical procedure in Europe and Japan, but not in North America. The reconstitution of immune function following removal of e.g. a monoclonal immunoglobulin with plasma exchange procedures, or immune reconstitution with HAART, is often accompanied by an exaggerated pathological inflammatory response termed immune reconstitution inflammatory syndrome (IRIS), also known as “restoration disease (IRD)”, “immune reconstitution syndrome (IRS)”, “immune recovery disease”, and “immune rebound illness”. As the immune system recovers, influx of cytotoxic and bystander lymphocytes eliminates infected oligodendrocytes and augments bystander inflammation. The immune system has been postulated to respond to a previously acquired opportunistic infection with an overwhelming inflammatory response that paradoxically renders symptoms of infection worse. Since IRIS has been found to occur in the absence of any apparent active infection, it has also been postulated to arise merely due to restoration of the previously suppressed inflammatory immune response due to reactivation of memory cells that had been previously activated by antigen exposure. IRIS typically leads to clinical deterioration, causing high disability and mortality. IRIS was first described in patients with HIV, however it is more common in MS patients treated with Natalizumab.

In HIV infected subjects IRIS typically develops within weeks or months (Post, M. J. D., et al., Am. J. Neuroradiol. (2013) 10.3174/ajnr.A3183). IRIS significantly negatively impacts the HIV infected population on HAART by increasing the number of procedures, number of hospitalizations, and the overall morbidity in this patient cohort (ibid.). Among JCV positive HIV infected patients that have been treated with HAART, it has been reported that 18% may develop IRIS (ibid.). In HIV negative patients on immunomodulatory therapy such as Natalizumab, PML-IRIS is reportedly more severe than in HIV infected patients due to the restored immune surveillance in the latter (ibid.).

IRIS is a robust inflammatory response, which may occur as a mild disease, but also as a life-threatening deterioration. A method according to the invention allows early prediction of the risk of PML occurrence and therefore provides time to adjust treatment before onset of PML. Thus occurrence of IRIS may be avoided and thereby a potential additional health/life risk be circumvented.

The method described above can likewise be used to diagnose the severity of PML in a subject that suffers from HIV infection or in a subject that is undergoing treatment with an α₄-integrin blocking agent, a LPAM-1 blocking agent and/or a VLA-4 blocking agent. In some embodiments in a method according to the invention as described above the subject from whom/which the sample originates is generally known to have HIV and PML. Detecting the level of CD62L and/or PSGL-1 expressing T cells in a sample from the subject and comparing the same to a threshold value may be carried out as above. A level of CD62L and/or PSGL-1 expressing T cells below a predetermined threshold value may indicate a condition where the subject is suffering from severe PML. In some embodiments in a method according to the invention as described above the subject from whom/which the sample originates is generally known to have HIV, but not PML. Such a method may be a method of assessing the risk of development of PML. In such an embodiment the subject may be suspected to be at risk of developing PML. A decreased level of one or both of CD62L and PSGL-1 expressing T cells in a sample from the subject relative to the threshold value indicates the risk of development of PML.

A method as described above may also be a method of assessing the occurrence of PML. In such an embodiment the subject from whom/which the sample originates is generally suspected to suffer from PML. A decreased amount of CD62L, PSGL-1 and/or LFA-1, relative to the threshold value, indicates the presence of PML. A method as described above may further in some embodiments be a method of assessing the chances of survival from PML in a subject. In such an embodiment the subject is generally known to have PML. A decreased level of CD62L and/or PSGL-1 expressing T cells, relative to the threshold value, may indicate low chances of survival of PML.

In addition, further biomarkers, already known or to be discovered, can be optionally used as secondary markers in the context of the present invention to assist the assessment of the risk of occurrence of PML of a subject such as a patient receiving one or more α₄-integrin blocking agents, LPAM-1 blocking agents and/or VLA-4 blocking agents. As explained above, further indicators that may be taken into account for diagnosing a risk of PML include the treatment duration, pretreatment with immunosuppressants, as well as the serum-positivity of JCV antibodies.

### Treatment and Administration of Compounds

The present invention also provides a method of treating a subject. The method includes administering an α₄-integrin blocking agent, LPAM-1 blocking agent and/or a VLA-4 blocking agent or an antiviral agent to the subject. The method further includes determining the expression of one or more biomarkers on one or more T cells from the subject, such as CD4⁺ T cells or CD8⁺ T cells. Such a biomarker is generally CD62L, PSGL-1 or LFA-1. In some embodiments the expression of one or more biomarkers on one or more T cells from the subject may be monitored. In some embodiments the method further includes determining, including monitoring, the migration of CD45⁺CD49d⁺ immune cells. In this regard the invention also relates to the use of an α₄-integrin blocking agent, a LPAM-1 blocking agent and/or a VLA-4 blocking agent or an antiviral agent in the manufacture of a medicament. As explained above, a subject receiving a treatment in accordance with the invention may be an immunocompromised individual. The subject may for instance have acute lymphoid leukaemia, chronic myeloid leukaemia, ulcerative colitis, Crohn's disease or HIV infection.

An α₄-integrin blocking agent, a LPAM-1 blocking agent and/or a VLA-4 blocking agent or an antiviral agent can be administered to a human patient per se, or in pharmaceutical compositions where they are mixed with other active ingredients, as in combination therapy, or suitable carriers or excipient(s). Exemplary routes include, but are not limited to, oral, transdermal, and parenteral delivery.

An α₄-integrin/VLA-4 blocking agent VLA-4 blocking agent can be used to treat a number of diseases and disorders, including multiple sclerosis, Crohn's disease, rheumatoid arthritis, meningitis, neuromyelitis optica, neurosarcoidosis, CNS vasculitis, chronic inflammatory demyelinating polyradiculoneuropathy (CIDP), encephalitis, transverse myelitis, tissue or organ graft rejection or graft-versus-host disease, chronic renal disease, CNS injury, e.g., stroke or spinal cord injury; chronic renal disease; allergy, e.g., allergic asthma; type 1 diabetes; inflammatory bowel disorders, e.g., ulcerative colitis; myasthenia gravis; fibromyalgia; arthritic disorders, e.g., rheumatoid arthritis, psoriatic arthritis; inflammatory/immune skin disorders, e.g., psoriasis, vitiligo, dermatitis, lichen planus; systemic lupus erythematosus; Sjogren's Syndrome; hematological cancers, e.g., multiple myeloma, leukemia, lymphoma; solid cancers, e.g., sarcomas or carcinomas, e.g., of the lung, breast, prostate, brain; and fibrotic disorders, e.g., pulmonary fibrosis, myelofibrosis, liver cirrhosis, mesangial proliferative glomerulonephritis, crescentic glomerulonephritis, diabetic nephropathy, and renal interstitial fibrosis. Any disease or pathological condition which has been treated or is known to be treatable by the blocking agent is part of the present invention. Accordingly, the treatment generally includes administering a therapeutically effective amount of an α₄-integrin/VLA-4 blocking agent or of an antiviral agent. Subjects may be first subjected to prior screening to determine whether the treatment would be suitable. For example, the screening may be based on the patient history, previous use of immunosuppressant, Expanded Disability Status Scale (EDSS) in case of multiple sclerosis patients, anti-JCV antibody status (JCV antibody seropositivity), MRI imaging studies, pre-infusion checklist for continuously worsening neurological symptoms, and other criteria commonly used.

Suitable routes of administration of compounds/agents used in the context of the present invention may, for example, include depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into a solid tumor, often in a depot or sustained release formulation. Furthermore, one may administer the drug in a targeted drug delivery system, for example, in a liposome coated with a blood-cell specific antibody. The liposomes will be targeted to and taken up selectively by the respective cells.

Pharmaceutical compositions that include the compounds of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers including excipients and auxiliaries that facilitate processing of the active compounds into preparations that can be used pharmaceutically. Proper formulation is dependent upon the route of administration chosen.

For injection, the agents of the invention may be formulated in aqueous solutions, for instance in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated.

Pharmaceutical preparations for oral use can be obtained by adding a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl-pyrrolidone (PVP).

If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system including benzyl alcohol, a non-polar surfactant, a water-miscible organic polymer, and an aqueous phase. The co-solvent system may be the VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: D5W) consists of VPD diluted 1:1 with a 5% dextrose in water solution.

This co-solvent system dissolves hydrophobic compounds well, and itself produces low toxicity upon systemic administration. Naturally, the proportions of a co-solvent system may be varied considerably without destroying its solubility and toxicity characteristics.

Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

A pharmaceutical composition also may include suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

For any compound used in the methods of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the desired activity). Such information can be used to more accurately determine useful doses in humans.

Toxicity and therapeutic efficacy of the compounds described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD₅₀ and ED₅₀. It may be desired to use compounds that exhibit high therapeutic indices. The data obtained from these cell culture assays and animal studies can be used in formulating a range of dosage for use in humans. The dosage of such compounds lies typically within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of the kinase. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen that maintains plasma levels above the MEC for 10-90% of the time, for example from about 30 to about 90%, such as from about 50 to about 90%.

In cases of local administration or selective uptake, the effective local concentration of the drug may not be related to plasma concentration. The amount of composition administered will, of course, be dependent on the subject being treated, on the subject's weight, the severity of the affliction, the manner of administration and the judgment of the prescribing physician.

A suitable composition may, if desired, be presented in a pack or dispenser device which may contain one or more unit dosage forms containing the active ingredient. The pack may for instance include metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the compound for human or veterinary administration. Such notice, for example, may be the labeling approved by the U.S. Food and Drug Administration or other government agency for prescription drugs, or the approved product insert.

Certain aspects of the present invention concern a method of treating a subject such as a patient. In some embodiments the treatment includes administering one or more antiretroviral compounds to the patient. In some embodiments of such a method the level of expression of PSGL-1 on T cells of the subject is measured/detected. The administration of the one or more antiretroviral compounds is stopped or continued, based on the level of expression of PSGL-1 on the subject's T cells. As explained above, the level of PSGL-1 expression on T cells can be used to assess the risk of occurrence or the occurrence of PML. A threshold value may be used as a decision threshold (supra). Hence, if a PSGL-1 expression level is detected that indicates that there is no elevated risk of PML occurrence, the treatment may be continued. If a PSGL-1 expression level is detected that indicates that there is an elevated risk of PML occurrence, the administration of the one or more antiretroviral compounds should be stopped. Stopping the administration of the one or more antiretroviral compounds may include terminating or adjourning administering the one or more antiretroviral compounds to the subject. In some embodiments stopping the administration of the one or more antiretroviral compounds includes administering one or more antiretroviral compounds that differ from that/those antiretroviral compound(s) previously administered to the subject.

In some embodiments of such a method the level of expression of CD62L on T cells of the subject is measured/detected. The administration of the one or more antiretroviral compounds is stopped or continued, based on the level of expression of CD62L on the subject's T cells. As explained above, the level of CD62L expression on T cells can be used to assess the risk of occurrence or the occurrence of PML. As explained above, a threshold value may be used as a decision threshold. Hence, if a CD62L expression level is detected that indicates that there is an elevated risk of PML occurrence, the administration of the one or more antiretroviral compounds should be stopped. If a CD62L expression level is detected that indicates that there is no elevated risk of PML occurrence, the treatment may be continued. In one embodiment the level of expression of both PSGL-1 and CD62L on T cells of the subject is measured/detected. The administration of the one or more antiretroviral compounds is stopped or continued, based on the level of expression of PSGL-1 and CD62L on the subject's T cells. If both a PSGL-1 expression level and a CD62L expression level are detected that indicate that there is no elevated risk of PML occurrence, the treatment may be continued. If a PSGL-1 expression level and/or a CD62L expression level is detected that indicates that there is an elevated risk of PML occurrence, the administration of the one or more antiretroviral compounds should be stopped. Again, stopping the administration of the one or more antiretroviral compounds may include terminating or adjourning administering the one or more antiretroviral compounds to the subject. Stopping the administration of the one or more antiretroviral compounds may include administering one or more antiretroviral compounds that differ from that/those antiretroviral compound(s) previously administered to the subject.

In addition, if a PSGL-1 and/or CD62L expression level is detected that indicates that a subject is at an elevated risk of PML occurrence, diagnosis with regard to PML may be intensified. As further explained below, MRI imaging may be employed to identify any area of demyelination. Further, cerebrospinal fluid may be analysed for the presence of JCV DNA, or blood or a brain sample may be analysed with regard to the presence of TNFR1 or TNF-α. If any of these diagnostic measures have previously been carried out on the subject, including carried out on a regular basis, if on the basis of PSGL-1 and/or CD62L expression levels, a subject is found to be at an elevated risk of developing PML, one or more such means of diagnosing PML may be carried out on a regular basis, including on a more frequent basis than previously done. As an illustrative example, it may be decided by the physician that every three months MRI imaging is carried out on the subject's brain.

In some embodiments the treatment includes administering an α₄-integrin blocking agent, a LPAM-1 blocking agent and/or a VLA-4 blocking agent to the subject. The method further includes measuring or detecting the level of expression of PSGL-1 on T cells of the subject. Based on the level of expression of PSGL-1 on the subject's T cells the administration of the α₄-integrin blocking agent, LPAM-1 blocking agent and/or VLA-4 blocking agent is stopped or continued. A threshold value may be used as a decision threshold (supra). If a PSGL-1 expression level is detected that indicates that there is no elevated risk of PML occurrence, the administration of the blocking agent may be continued. If a PSGL-1 expression level is detected that indicates that there is an elevated risk of PML occurrence, the administration of the blocking agent should be stopped. In some embodiments measures are taken to remove the α₄-integrin, LPAM-1 and/or a VLA-4 blocking agent from the subject's plasma if an elevated risk of PML occurrence has been determined. As explained above plasma exchange or immunoadsorption may be carried out in this regard. In some embodiments stopping the administration of a blocking agent means that therapy with an α₄-integrin blocking agent, LPAM-1 blocking agent and/or VLA-4 blocking agent is entirely stopped, i.e. no alternative blocking agent is administered instead of the previously administered α₄-integrin, LPAM-1 or VLA-4 blocking agent. In some embodiments an α₄-integrin blocking agent, LPAM-1 blocking agent and/or VLA-4 blocking agent is an immunoglobulin or a proteinaceous binding agent with immunoglobulin-like functions. In such an embodiment stopping the administration of a blocking agent means that therapy with an immunoglobulin or a proteinaceous binding agent that is an α₄-integrin blocking agent, LPAM-1 blocking agent and/or VLA-4 blocking agent is entirely stopped, i.e. no alternative immunoglobulin or a proteinaceous binding agent is administered instead of the previously administered immunoglobulin or a proteinaceous binding agent. In such an embodiment an α₄-integrin blocking agent, LPAM-1 blocking agent and/or VLA-4 blocking agent that differs from an immunoglobulin or a proteinaceous binding agent may be administered, for instance a low molecular weight compound. Entirely ending or adjourning therapy with an α₄-integrin blocking agent, a LPAM-1 blocking agent and/or a VLA-4 blocking agent may assist reconstitution of the subject's immune surveillance. It is further noted in this regard that a subject suffering from MS and under therapy with an α₄-integrin blocking agent, LPAM-1 blocking agent and/or VLA-4 blocking agent is often a subject that/who did not respond to a first-line therapy such as interferon-β or glatiramer acetate. Beginning such a therapy as a substitute of α₄-integrin, LPAM-1 and/or VLA-4 blocking agent therapy may therefore only have a low chance of improving the subject's condition.

In some embodiments a method of treating a subject, which includes administration of an α₄-integrin blocking agent, a LPAM-1 blocking agent and/or a VLA-4 blocking agent, further includes measuring the level of expression of CD62L on T cells of the subject. The administration of the α₄-integrin blocking agent, LPAM-1 blocking agent and/or VLA-4 blocking agent is stopped or continued based on the level of expression of PSGL-1 and CD62L on the subject's T cells. If one or both of a PSGL-1 expression level and a CD62L expression level is detected that indicates that there is an elevated risk of PML occurrence, the administration of the respective blocking agent should be stopped, otherwise the treatment may continue.

As explained above, the assessment/evaluation of the need to discontinue or not discontinue the administration of an α₄-integrin blocking agent, a LPAM-1 blocking agent and/or a VLA-4 blocking agent or of one or more antiretroviral compounds is typically based on a comparison of the subject's expression level of PSGL-1 and/or CD62L with a threshold level. The determination whether to stop the treatment of the subject or not can be based on comparing the level of expression with a reference. The reference may be derived from one or more patients known to have suffered from PML or other complications, or one or more patients known to have not suffered from PML or other complications (supra). As an example, a reference value or level can be gathered from control subjects. Expression levels PSGL-1 and/or CD62L from the control subjects using any suitable method may be recorded over a period of time, such as over a period selected in the range of about 2-3 years. Average expression levels, standard deviation, and relative standard deviation at given times can be calculated for the control subjects to determine a range of expression levels associated with the control subjects. When a test result from a subject to be evaluated is collected, it will be compared to the reference value. Statistical differences between the test result and the reference will be determined to identify significant variances between the respective expression levels. Based on PSGL-1 and/or CD62L expression, a physician is able to assess whether to continue, restart or stop a treatment with an α₄-integrin blocking agent, a LPAM-1 blocking agent and/or a VLA-4 blocking agent or of one or more antiretroviral compounds. The information provides significant information to the physician regarding the risk associated with the treatment, so that informed benefit-risk decisions can be taken accordingly.

In some embodiments a method of treating a subject, whether including administration of an α₄-integrin blocking agent, a LPAM-1 blocking agent and/or a VLA-4 blocking agent or administration of one or more antiretroviral compounds, further includes measuring the level of expression of LFA-1 on the subject's T cells. As will be apparent from the forgoing, the above explanations with regard to CD62L apply mutatis mutandis to of LFA-1. Thus in one embodiment the administration of the α₄-integrin, LPAM-1 and/or a VLA-4 blocking agent or of the one or more antiretroviral compounds is stopped or continued based on the level of expression of PSGL-1 and LFA-1 on the subject's T cells. If one or both of a PSGL-1 expression level and a LFA-1 expression level is detected that indicates that there is an elevated risk of occurrence of PML or at least some aspects of PML, the administration of the VLA-4 blocking agent or of the one or more antiretroviral compounds should be stopped, otherwise the treatment may continue. In one embodiment the administration of the α₄-integrin, LPAM-1 and/or a VLA-4 blocking agent or of the one or more antiretroviral compounds is stopped or continued based on the level of expression of PSGL-1, CD62L and LFA-1 on the subject's T cells. If one or more of a PSGL-1 expression level, a CD62L expression level and a LFA-1 expression level is detected that indicates that there is an elevated risk of PML occurrence, the administration of the respective blocking agent or of the one or more antiretroviral compounds should be stopped, otherwise the treatment may be continued.

A method of treating a subject with a retroviral infection such as HIV according to the invention may include administering a combination of anti-retroviral compounds to the subject over a period of time, followed by a discontinuation of the administration for a period of time (supra). As explained above, discontinuation of the administration typically includes administering an alternative combination of anti-retroviral compounds to the subject. The method aims at avoiding the additional occurrence of PML. Generally one or more reverse transcriptase inhibitors and optionally a protease inhibitor are administered to the subject. In some embodiments treating the subject with a retroviral infection includes determining, including monitoring, the expression level of CD62L and/or PSGL-1 on T cells in or from a sample of the subject. The method of treating the subject generally includes administering a therapeutically effective amount of each reverse transcriptase inhibitor and/or protease inhibitor used.

Any suitable combination of antiretroviral compounds may be used in the context of the present invention. Typically three or more antiretroviral compounds are being administered simultaneously. One of the antiretroviral compounds may be a nucleoside reverse transcriptase inhibitor such as Zidovudine (AZT), Didanosine (ddI), Zalcitabine (ddC), Stavudine (d4T), Lamivudine (3TC), Emtricitabine, Abacavir, Amdoxovir, Apricitabine or Elvucitabine. One of the antiretroviral compounds may be a nucleoside/nucleotide reverse transcriptase inhibitor such as Tenofovir, Tenofovir disoproxil fumarate (DF) or Adefovir. One of the antiretroviral compounds may also be a protease inhibitor such as Indinavir, Saquinavir hard gel, Ritonavir, Nelfinavir, Fosampernavir, Lopinavir, Atazanavir, Tipranavir or Darunavir. Further, one of the antiretroviral compounds may be a non-nucleoside reverse transcriptase inhibitor such as Nevirapine, Delaviridine, Efavirenz, Etravirine or Rilpivirine. One of the antiretroviral compounds may also be a so called “entry inhibitor”, i.e. a compound that blocks the entry of the retrovirus into a cell. Two illustrative examples of entry inhibitors are Enfuvirtide, which blocks the fusion of the HIV envelope to the cell membrane, and Maraviroc, which is a CCR5 co-receptor antagonist. Further, one of the antiretroviral compounds may be an integrase inhibitor, i.e. a compound that inhibits the viral integrase enzyme, which is required for viral replication. An illustrative example of an integrase inhibitor is Raltegravir.

As explained above, the present invention further provides a method of treating a subject that/who is in an immunocompromised condition, for instance having an autoimmune disease, which may be a demyelinating disease. The method includes administering an α₄-integrin blocking agent, a LPAM-1 blocking agent and/or a VLA-4 blocking agent to the subject. Typically the method also includes monitoring the expression of at least one biomarker on T cells, with the monitoring being carried out on a sample from the subject. A respective biomarker may be CD62L, PSGL-1 and/or LFA-1. The method may also include determining the migratory capacity of CD45⁺CD49d⁺ immune cells.

Typically the treatment of a subject that/who is in an immunocompromised condition includes administering a therapeutically effective amount of an α₄-integrin blocking agent, a LPAM-1 blocking agent and/or a VLA-4 blocking agent. The blocking agent may, for example, be administered intravenously. For Natalizumab, the dose may be 1 to 6 mg per kilogram of body weight. In one embodiment, a standard does of 300 mg Natalizumab diluted with 100 ml 0.9% sodium chloride is injected intravenously once every four weeks. The dose may be repeated at intervals from two to eight weeks. For example, a treatment regimen may include 3 mg Natalizumab per kg body weight repeated at about a four week interval. A skilled person in the art is capable of determining the therapeutic effective amount.

A subject may be first subjected to prior screening to determine whether the planned treatment would be suitable. For example, such a screening may be based on the patient history, previous use of immunosuppressant, Expanded Disability Status Scale (EDSS) in case of multiple sclerosis patients, anti-JCV antibody status (JCV antibody seropositivity), MRI imaging studies, pre-infusion checklist for continuously worsening neurological symptoms, and other criteria commonly used. A brain biopsy may also be performed to determine whether characteristic features of PML, known to the practitioner in the field, can be found.

In some embodiments of a method according to the invention the presence or absence of anti-JCV immunoglobulins in blood of the subject is determined. The presence of anti-JCV immunoglobulins in blood of the subject indicates that the subject may potentially develop PML. If anti-JCV immunoglobulins are detected in blood of the subject, the level of PSGL-1, LFA-1 and/or CD62L may be determined. The absence of anti-JCV immunoglobulins in blood of the subject typically indicates that the subject is at no elevated risk of developing PML. The absence of anti-JCV immunoglobulins in blood of the subject may indicate that levels of anti-JCV immunoglobulins are below the detection limit of the used technique. In such a case an alternative immunoglobulin test may be used. In such a case MRI imaging may be employed, or the presence of JCV DNA in cerebrospinal fluid, the presence of TNFR1 or of TNF-α in blood or in a brain sample may be determined. In some embodiments of a method according to the invention the presence or absence of JCV DNA in blood of the subject is determined. It is to be noted that the presence of JCV DNA in blood is correlated with immunosuppression rather than with PML. Nevertheless in the context of a subject who/that is suffering from a retroviral disease, e.g. HIV, MS or Crohn's disease, the absence of JCV DNA in blood my indicate that the subject is not at an elevated risk of developing PML.

In some embodiments of a method according to the invention the presence or absence of JCV DNA in the cerebrospinal fluid of the subject is determined. If JCV DNA is detected in cerebrospinal fluid of the subject, the level of PSGL-1, LFA-1 and/or CD62L may be determined. It is to be noted that a false positive JCV test of JCV DNA in cerebrospinal fluid occurs in 1-4% of HIV positive subjects. Furthermore, JCV DNA is often only detectable in cerebrospinal fluid after onset of PML. In some embodiments of a method according to the invention the level of TNFR1 or of TNF-α in blood or in a brain sample of the subject is determined. If detectable levels of TNFR1 or of TNF-α are found in a brain sample the subject may potentially be at an elevated risk to develop PML. In this case the level of PSGL-1, LFA-1 and/or CD62L may be determined. If elevated levels of TNFR1 or of TNF-α are found in blood the subject may potentially be at an elevated risk to develop PML. In this case the level of PSGL-1, LFA-1 and/or CD62L may be determined.

Typically a subject undergoing α₄-integrin, LPAM-1 and/or a VLA-4 blocking agent treatment is tested to determine the expression level of a biomarker as disclosed in this document, e.g. the expression level of CD62L and/or PSGL-1 on T cells in or from a sample of the subject. As a further example, the migratory capacity of CD45⁺CD49d⁺ immune cells may be determined. A method according to the invention may also include any other molecule or effect that can be used to indirectly indicate the level of such biomarker. One or more samples from the subject are collected and analyzed. In some embodiments the one or more samples are sent to a central testing facility to ensure that the analysis of phenotype and function can be carried out under standardized conditions. Samples may be taken and tested prior to the treatment and then regularly after the treatment begins, such as monthly, bimonthly, quarterly, every six months, and yearly. The routine assessment for PML provides timely information regarding the safety issues related to the treatment. In one embodiment, the samples are taken at month 1, every 3 months until the first year, and then every 6 months thereafter.

In some embodiments treating the subject undergoing treatment with an α₄-integrin blocking agent, a LPAM-1 blocking agent and/or a VLA-4 blocking agent includes determining, including monitoring, the expression level of CD62L, PSGL-1 and/or LFA-1 on T cells in or from a sample of the subject. If any indication is found that suggests an increased risk of PML occurrence or of the occurrence of another complication, or renders such complication more likely than in other subjects, further tests may be carried out. Subjects showing compromised immune surveillance should be clinically monitored very closely. The physician may test the patient for further biomarkers such as those provided in the present invention or known biomarkers, such as anti-JCV antibody status or other clinical or MRI criteria. Based on the information, the practitioner will assess whether to continue, restart or stop the treatment with an α₄-integrin blocking agent, a LPAM-1 blocking agent and/or a VLA-4 blocking agent. The information provides significant information to the physician regarding the risk associated with the treatment, so that informed benefit-risk decisions can be made accordingly. As an illustrative example, the monitoring may at the beginning include only determining the level of CD62L expression. When the result indicates a low expression level, compared to the reference value as described above, the LFA-1 expression level and/or the migration capacity of T cells may be tested. When the result does not indicate a drop in CD62L levels, including any alteration, for instance by showing a normal or inconspicuous expression level of CD62L, compared to the reference value, the level of PSGL-1 expression on T cells from the sample may be analysed. As a further illustrative example, the monitoring may initially include only determining the level of PSGL-1 expression. When the result does not indicate a drop in CD62L levels, including any change, for instance by showing a normal or inconspicuous expression level of PSGL-1, compared to the reference value as described above, the CD62L expression level on T cells may be determined.

In some embodiments, a reference value or level can also be gathered from subjects who suffered from PML as a result of α₄-integrin, LPAM-1 and/or a VLA-4 blocking agent treatment. Expression levels of the biomarkers from the PML patients are recorded over a period of time, such as over 2-3 years. Average expression levels, standard deviation, and relative standard deviation at given times are calculated for the individuals to determine a range of expression levels associated with subjects having PML. When a test result from an individual to be evaluated is collected, it will be compared to the reference value. Statistical differences between the test result and the reference will be determined to identify significant variances in between.

Accordingly, determining the expression level of CD62L, PSGL-1 and/or LFA-1 can be used to stratify a subject undergoing or about to undergo treatment with an α₄-integrin blocking agent, a LPAM-1 blocking agent and/or a VLA-4 blocking agent for suspension of the treatment. Determining the expression level of CD62L and/or PSGL-1 can also be used to stratify a subject undergoing HAART for suspension of the respective HAART, which may include carrying out an alternative HAART. As explained above, stratification may be based on the assessment of the risk of a subject to of develop PML. As also explained above, in some embodiments of a method of the invention a binding partner specific for CD62L, PSGL-1 and/or LFA-1 is used to screen risk patients which have higher susceptibility to PML.

With regard to human individuals, the use of biomarkers for stratification of patients is a procedure well established in the art. This procedure includes or consists of linking one or more patient subpopulations, characterized by a certain feature, in the context of the present invention the expression level of a particular protein or migratory capacity of cells, to a particular treatment. The general aim of stratification is to match patients with therapies that are more likely to be effective and safe. In a more general context stratifying patients may include evaluation of patient history and physical assessment, combined with laboratory tests on the basis of a method of the present invention, and clinical observation. It is understood that stratifying patients is only feasible if multiple treatment options with heterogeneous responses for the disease exist. In the context of the present invention HIV therapy may be adjusted or treatment with an α₄-integrin blocking agent, a LPAM-1 blocking agent and/or a VLA-4 blocking agent be suspended for a certain period of time, such as one or more months. A general overview of patient stratification and stratified medicine has been given by Trusheim, M. R., et al., Nature Reviews Drug Discovery (2007) 6, 4, 287-293.

### Migration of Immune Cells

As already noted above, in some embodiments a method according to the invention includes determining, including monitoring, the migration of immune cells expressing CD45 and CD49d. In some of these embodiments the subject of whom the sample has been obtained is undergoing treatment with an α₄-integrin blocking agent, a LPAM-1 blocking agent and/or a VLA-4 blocking agent. Generally, CD45d is expressed on all leukocytes, and CD49d is expressed on T cells, B cells, monocytes, eosinophils and basophils. In some embodiments the immune cell to be tested is T cell. In one embodiment the T cell is a CD4⁺ T cell. In one embodiment the T cell is a CD8⁺ T cell.

Migratory capacity of immune cells is a prerequisite for immune reactions. A respective method of the invention which can be used to evaluate a subject's immune competence and risk status to develop PML. Any technique that is suitable for determining the migratory capacity of an immune cell can be used. This can be done using any known techniques in the art. In some embodiments a chemotaxis assays is employed. Such assays are based on the functional migration of cells induced by a compound, and can be used to assess the binding and/or chemoattractant effect of e.g. ligands, inhibitors, or promoters. The use of an in vitro assay is illustrated in the Examples and also disclosed in U.S. Pat. No. 5,514,555. In some embodiment chemotaxis assay determines the migration across endothelium into a collagen gel (described in Kavanaugh et al, J. Immunol (1991) 146, 4149-4156). Such assay may involve the use of a transwell-based set-up. In some embodiments a chemoattractant is dissolved in the medium on one side of a migratory barrier such as a polymeric gel. On the other side of the migratory barrier the cells of the sample from the patient are positioned. The migratory barrier is porous to a certain extent so that the cells of interest such as T cells are able to migrate through the same. The pores of the porous migratory barrier further allow the passage of chemoattractant molecules, so that a diffusion gradient forms, which can be detected by the cells of interest. As a result the cells are attracted to migrate across the migratory barrier. Typically the cells are allowed to migrate in the experimental setup for a certain, e.g. predetermined, period of time, whereafter the number of migrated cells and/or the migration distance is being determined. For this purpose the migratory barrier may be analysed under a microscope. The cells may also be stained before starting the chemotaxis assay and their position be determined according to the signals obtained from the stain.

In one embodiment, the migratory capacity is compared to that obtained from the same patient prior to the treatment with α₄-integrin, LPAM-1 and/or VLA-4 blocking agent. The value obtained can be set to 100%. After treatment is initiated, a drop in immune cell migration can be observed and compared. The migration at a given time point can be characterized by an average (mean) value coupled with a standard deviation value. Cell migration in a subject may be considered different when it is more than one standard deviation different from the average value (supra). The reference value may be defined as the mean minus 1 standard deviation. When the difference falls below the reference value, it may be indicative of an increased risk for PML occurrence in the subject. The above said with regard to a threshold value in this regard applies mutatis mutandis.

As an illustrative example the following table provides an exemplary reference level for immune cell migration that may be used. In this instance, migration of CD3⁺ T cells over endothelium has been monitored over a period of time.

TABLE 3
Exemplary reference values for migration of CD3⁺ T cells

| | % of migrated CD3⁺ T cells in | | |
|---|---|---|---|
| | relation to untreated patients | | reference level (mean % of |
| | (set to 100%) | | migrated CD3⁺ T cells |
| Month | (mean (standard deviation)) | | minus 1 standard deviation) |
| 0 (before | 100.0 | (none) | |
| treatment) | | | |
| 1 | 62.7 | (27.5) | 35.2 |
| 3 | 38.8 | (7.0) | 31.8 |
| 6 | 11.1 | (11.3) | 0 |
| 12 | 31.3 | (22.3) | 9.0 |
| 15-20 | 71.7 | (38.9) | 32.8 |
| 21-25 | 104.7 | (61.8) | 42.9 |
| 26-30 | 61.8 | (36.9) | 24.9 |
| 31-35 | 35.7 | (22.7) | 13.0 |
| 36-40 | 57.5 | (25.7) | 31.8 |
| 41-45 | 57.0 | (22.6) | 34.4 |
| 46-50 | 104.6 | (48.8) | 55.8 |
| 51-55 | 119.8 | (45.6) | 74.2 |

Immune cells have a basic capacity to migrate over cellular barriers and permeable membranes. The inventors have found that a lack or reduced CD62L expression and/or lack or reduced PSGL-1 expression is associated with a strongly reduced migratory capacity. A migration assay used in a method according to the present invention may involve the use of a permeable membrane. The membrane may be any membrane commonly used in the field, such as polycarbonate (PC), polyester (PET), and collagen-coated polytetrafluoroethylene (PTFE) membrane, which are available commercially (for example Transwell® membrane). A migration assay over a blank permeable membrane, i.e. without cells, may be used for such assessment. In some embodiments a migratory assay involves the use of cells. Cells such as endothelial cell or cell lines are within the scope of the present invention. Examples of suitable cells include, but are not limited to, cells of the HMEC-1 cell line, of the human brain endothelial cell line HCMEC/D3, of the murine cell lines mHEVa and mHEVc, of the mouse aortic vascular endothelial cell line MAEC, of the Mouse Cardiac Endothelial Cell line MCEC, the c-end cell line, and cells of the immortal hybridoma cell line EA.hy926. As a further example of suitable cells, human umbilical vein endothelial cells (HUVEC) may be used, which are primary cells isolated from the human umbilical vein of a donor. In some embodiments primary human choroid plexus-derived epithelial cells are employed. In some embodiments primary human brain microvasculary endothelial cells are used.

As indicated above, any number of steps of a method according to a method of the invention, including the entire method, may be performed in an automated way—also repeatedly, using for instance commercially available robots. Computer executable instructions may for instance control data analysis or control mechanical courses of movements employed in a method according to the invention. As an illustrative example, the method may be an in vitro screening method, for example carried out in multiple-well microplates (e.g. conventional 48-, 96-, 384- or 1536 well plates) using automated work stations. The method may also be carried out using a kit of parts, for instance designed for performing the present method. As a further illustrative example, in cell sorting one or more steps may be initiated, or cell sorting parameters may be adjusted, using a series of computer executable instructions. Such computer executable instructions may be residing on a suitable computer readable medium. Suitable computer readable media may include volatile (e.g. RAM) and/or non-volatile (e.g. ROM, disk) memory, carrier waves and transmission media (e.g. copper wire, coaxial cable, fibre optic media). Exemplary carrier waves may take the form of electrical, electromagnetic or optical signals conveying digital data streams along a local network or a publically accessible network such as the Internet.

### Kit

Reagents needed or useful in the context of the present invention may be provided in the form of a kit. Such a kit may in particular include means for detecting one or more biomarkers as provided in the present invention. Means for detecting a biomarker are known in the art, and include, for example, the use of a binding partner such as an immunoglobulin or a proteinaceous binding molecule with immunoglobulin-like functions, which optionally is detectably labeled. As explained above the binding partner may be used together with a detection agent that binds to the biomarker and/or the binding partner. In one embodiment the kit may include a CD62L specific binding partner, and optionally a LFA-1 binding partner. In one embodiment the kit may include a PSGL-1 binding partner. In one embodiment the kit may include both a PSGL-1 specific binding partner and a CD62L specific binding partner. The kit may further include a CD3 specific binding partner. In some embodiments the kit may further include a CD4 specific binding partner and/or a CD8 specific binding partner. In some embodiments the kit may include a container that has a first immunoglobulin or a proteinaceous binding molecule with immunoglobulin-like functions. The first immunoglobulin or proteinaceous binding molecule is capable of specifically binding to PSGL-1. The kit may also include a container that has a second immunoglobulin or a proteinaceous binding molecule with immunoglobulin-like functions. In some embodiments the kit may also include a reagent that allows the detection of a detectable label, which is coupled to a binding partner of PSGL-1, CD62L, LFA-1, CD3, CD4 and/or CD8. As an illustrative example, the detectable label may be an enzyme and the reagent may be a substrate of the enzyme. The substrate may for instance be converted by such enzyme into a product that emits a signal such as a fluorescent or a colour signal. In some embodiments the kit may include a multi-specific binding partner directed to CD3, CD62L and LFA-1, optionally together with a detection agent. A multi-specific binding partner may for instance be directed to any two of CD3, CD62L, PSGL-1 and LFA-1. In one embodiment the kit includes components for setting up a method of detecting CD3 and CD62L. In one embodiment the kit includes components for carrying out a method of detecting CD3 and PSGL-1. In one embodiment the kit includes components for carrying out a method of detecting CD62L and PSGL-1. In some embodiments the kit includes an immunoglobulin or a proteinaceous binding molecule with immunoglobulin-like functions, or any other binding partner directed to the protein or mRNA of CD11a, and a binding partner directed to the protein or mRNA of CD18. Such a kit may also include a binding partner directed to the protein or mRNA of Runx3.

A respective kit may furthermore include means for immobilising the binding partner to a surface. As explained above, a nucleic acid binding partner included in the kit may have a moiety that allows for, or facilitates, an immobilisation on a surface.

The kit may further include instructions and/or imprint indicating that a patient is to be stratified by a method described herein; and/or instructions regarding how to carry out a method as defined herein. It may also include positive and/or negative controls which allow a comparison to the control. The kit shall enable the assessment of a patient's treatment progress and the risk of PML occurrence. A respective kit may be used to carry out a method according to the present invention. It may include one or more devices for accommodating the above components before, while carrying out a method of the invention, and thereafter.

Provided is also the use of a kit that includes components to be employed in a PSGL-1 binding assay, and optionally components to be employed in a CD62L and/or LFA-1 binding assay to determine the immune competence of a subject. The subject may be undergoing a treatment that includes an α₄-integrin blocking agent, a LPAM-1 blocking agent and/or a VLA-4 blocking agent or one or more anti-retroviral compounds. The binding assay may include a PSGL-1 binding partner and optionally a CD62L and/or a LFA-1 binding partner as described above. The kit allows the assessment of the risk for PML during the course of the treatment. Thereby the physician can determine whether to stop, continue, or resume the treatment of VLA-4 blocking agent or one or more anti-retroviral compounds, or to make any other suitable adjustment of a respective treatment regimen.

The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the appending claims. In addition, where features or aspects of the invention are described in terms of Markush groups, those skilled in the art will recognize that the invention is also thereby described in terms of any individual member or subgroup of members of the Markush group.

In order that the invention may be readily understood and put into practical effect, particular embodiments will now be described by way of the following non-limiting examples.

### EXAMPLES

The examples illustrate techniques that can be used in methods according to the invention as well as exemplary embodiments of determining the level of T cells that express L-CD62L, LFA-1 and/or PSGL-1. Studies in recent years have come up with three statistical observations of predicting an MS patient's overall risk to later develop PML when treated with Natalizumab, but there is no possibility yet to measure an individual's PML risk. This, however, is urgently needed to facilitate the treatment decisions of clinicians and patients alike, as many patients opt for continuing treatment with Natalizumab even when their statistical risk to develop PML rises up to roughly 1:120 with all three risk factors present. As compromised immune surveillance has long been a hypothesis to explain the occurrence of an opportunistic infection, the inventors designed a study to include a variety of adhesion molecules on the surface of immune cell subpopulations. Patients analysed included inter alia MS patients under long-term therapy with Natalizumab, MS patients before escalation to Natalizumab, but with diverse prior immunemodulatory treatments such as e.g. Glatiramer Acetate, Interferon-β, Azathioprine, or Methotrexate, HIV infected patients during the CDC stadium B1-C2, and HIV/AIDS patients during the CDC stadium C3.

The obtained data show that two surface molecules were strongly downregulated on T cells of patients who either would later develop PML or, in the case of HIV, had recently developed PML. These molecules were CD62L, and PSGL-1, both of them members of the selectin family. In addition, LFA-1 was likewise strongly downregulated on T cells of MS patients under Natalizumab therapy before occurrence of PML. These markers are therefore risk predictors, because their expression pattern during Natalizumab therapy differed. The expression of PSGL-1 rose continuously during long-term treatment with Natalizumab, whereas the expression of L-Selectin was stable during that time frame, meaning that PSGL-1 should be considered an ideal candidate for risk prediction the longer the therapy lasts and especially on CD8⁺ T cells, as the difference between the non-risk patients and the risk patients increased over time. However, at the start of therapy, PSGL-1 showed only a negligible difference in risk patients, whereas L-Selectin expression was already strongly reduced in some of the later risk patients (up to 45 months before PML onset).

As the expression of L-Selectin was usually higher on CD4⁺ T cells (because in contrast to CD8⁺ T cells, there are no CD4⁺CD62L⁻CD45RA⁺ cells), it may in some cases be advantageous to use CD4⁺ T cells to determine risk when using L-Selectin. As an additional note, one patient only showed the downregulation of L-Selectin, but not PSGL-1 before PML. Therefore, performing the combined measurement of both molecules (L-Selectin and PSGL-1) on CD4⁺ and CD8⁺ T cells may in some embodiments be advantageous to assess the risk for PML development, and to take a decision on changing a therapeutic regimen.

### Subjects Treated with Natalizumab

The status and longitudinal development, as well as function of major peripheral and CSF immune populations in patients under long-term treatment with Natalizumab was assessed. Focus of the following experiments underlying these examples was finding changes in the immune status of patients to possibly predict the occurrence of PML by assessing the impact of Natalizumab on T-cell function in combining immune phenotyping with functional in vitro and ex vivo assays.

Natalizumab, a humanized IgG4 antibody against the α-chain of VLA-4 (α4, CD49d), has been approved for the treatment of active relapsing-remitting Multiple Sclerosis (RRMS) since 2006. Long-term treatment with Natalizumab is associated with severe side effects, above all the development of progressive multifocal leukencephalopathy (PML). In addition to duration of treatment, previous immunosuppressive therapy (Panzara, M. A., et al., Multiple Sclerosis (2009) 15, 9, S132-S133) as well as the presence of JC virus, as ascertained by the presence of anti-JCV antibodies in serum, contribute to the risk of developing Natalizumab-associated PML (Bloomgren, G., et al., The New England Journal of Medicine (2012) 366, 20, 1870-1880). When all these risk factors are present, the statistical risk of PML can be as high as 1:120 (Clifford, D. B., et al., Lancet Neurology (2010) 9, 4, 438-446; Bloomgren et al., 2012, supra). While it is still unclear why and how (long-term) treatment with anti-CD49d contributes to the development of PML (Tan and Koralnik, 2010, supra), a multifactorial scenario is likely, including impaired immune surveillance and active JC virus replication (Schwab, N., et al. Multiple sclerosis [Houndmills, Basingstoke, England] (2012) 18, 3, 335-344; Schwab, N., et al. Neurology (2012) 78, 7, 458-467).

114 patients with the diagnosis of clinically definite active RRMS according to the 2005 revised McDonald diagnostic criteria (Polman, C. H. et al., Ann Neurol (2005) 58, 840-846) were enrolled in this study. 67 MS patients had continuously been treated with Natalizumab for 18-66 months, 21 MS patients received baseline immune-modulatory treatments (Interferons, Glatiramer acetate) and 26 MS patients were untreated and clinically as well as MRI-stable (cf. also Table 1, supra, for 22 of these patients). Age and sex-matched healthy donors (HD) with no previous history of any neurological or immune-mediated diseases served as controls. Furthermore, samples were available from different therapy-associated PML conditions: Natalizumab-associated (n=13), Rituximab-associated (n=1) and Efalizumab-associated (n=1). Six cases of HIV-associated PML served as additional controls. In 6 of the 13 Natalizumab-associated PML cases pre-PML samples were available (FIG. 14). The Table depicts average±standard deviation (if applicable).

The study was approved by the local ethics committee (Ethik-Kommission der medizinischen Fakultät der Universität Würzburg, registration number 155/06; Ethik-Kommission der Ärztekammer Westfalen-Lippe and der Medizinischen Fakultät der Wesfälischen Wilhelms-Universität, registration number: 2010-245-f-S) and informed written consent was obtained from all participants. This study was performed according to the Declaration of Helsinki.

Data shown in FIG. 1D, FIG. 11, FIG. 12 and FIG. 13 are based on a smaller group of patients. 52 patients with the diagnosis of clinically definite active RRMS according to the 2005 revised McDonald diagnostic criteria were enrolled. Analyzed MS patients had been treated continuously with Natalizumab for 18-50 months and were stable by assessment of clinical and MRI parameters. 18 patients among this cohort underwent analysis of CSF in parallel to assessment of peripheral blood. 39 patients were followed longitudinally from treatment initiation. Two patients developed PML after 26 or 29 months, respectively. 45 age and sex-matched healthy donors (HD) with no previous history of neurological or immune mediated diseases served as controls. Furthermore, 22 untreated MS patients served as controls (Table 1, supra). PBMC from patients suffering from PML (HIV⁺ (n=4), Natalizumab-associated (n=3), Rituximab-associated (n=1), Efalizumab-associated (n=1)) were used as additional controls.

Peripheral blood (n=52) and cerebrospinal fluid (n=18) from patients under Natalizumab therapy (≧18 months) were analyzed using flow cytometry and in vitro transendothelial migration assays.

Data shown in FIG. 3 are based on a group of patients of yet different size. 78 patients with the diagnosis of clinically definite active RRMS were included. Analyzed MS patients had been treated continuously with Natalizumab for 18 to 60 months and were stable by assessment of clinical and MRI parameters. Five patients developed PML. In addition, samples were obtained from 30 patients with the diagnosis of clinically definite active RRMS before treatment with Natalizumab. 73 age and sex-matched healthy donors with no previous history of neurological or immune mediated diseases served as controls.

### HIV Patients

Samples of 14 HIV patients at different stadiums of HIV infection were analysed. Three additional HIV patients were suffering from PML. Samples of the 73 age and sex-matched healthy donors with no previous history of neurological or immune mediated diseases (supra) were included as controls.

### Isolation of PBMC and Flow Cytometric Analysis

Peripheral blood mononuclear cells (PBMC) freshly isolated from EDTA blood were isolated by density gradient centrifugation using lymphocyte separation medium (PAA Laboratories, Pasching, Austria) as described previously in Schwab et al J. Immunol. (2010) 184, 9, 5368-5374, incorporated herein by reference in its entirety. Flow cytometry analysis of CSF was performed as described in Schwab et al. Multiple Sclerosis (2009) 15, S275-S275, incorporated herein by reference in its entirety. In case of conflict of a document incorporated by reference, the present specification, including definitions, will control. Cells were then typically cryopreserved in freezing medium (50% RPMI 40% FCS 10% DMSO).

Ex vivo isolated, cultured or thawed cells were washed with FACS®-buffer (phosphatebuffered saline (PBS) supplemented with 0.1% bovine serum albumine (BSA) and 0.1% NaN₃) or staining buffer (phosphate-buffered saline (PBS) supplemented with 0.1% bovine serum albumine (BSA) and 200 mM EDTA). Cells were subsequently stained with fluorescence-labeled monoclonal antibodies (Mab) together with blocking mouse IgG (Sigma-Aldrich, Hamburg, Germany) at 4° C. for 30 min or at room temperature for 15 min. After washing once with staining buffer, cells were immediately measured on a FACSCalibur (BD Biosciences, Heidelberg, Germany) and Gallios™ Flow Cytometer (Beckman Coulter, Krefeld, Germany) and analyzed using FlowJo (Tree Star, Ashland, Oreg., USA) and Kaluza (Beckman Coulter) software. It should be noted that the presence of CD62L on the cell surface tends to be unstable, so that the staining buffer cannot contain sodium azide and measurement needs to take place immediately after the staining procedure.

In particular, LFA-1 protein was stained for CD11a, the α-chain of LFA-1. VLA-4 was stained for CD49d (the α-chain of VLA-4), as CD49d is the precise molecule blocked by Natalizumab.

Monoclonal immunoglobulins used in these Examples were anti-CD62L (DREG-56, BioLegend), anti-CD3 (UCHT1, Beckman Coulter), anti-CD4 (13B8.2, Beckman Coulter), anti-CD8 (B9.11, Beckman Coulter), anti-CD11a (HI111, BD Pharmingen), anti-CD14, (MoP9, BD Biosciences), anti-CD19 (HIB19, BD Biosciences), anti-CD45 (J33, Beckman Coulter), anti-CD45RA (HI100, Beckman Coulter), anti-CD56 (NCAM 16.2, BD Biosciences), anti-CD49d (9F10, Biolegend) and anti-CD197 (3D12, BD Biosciences).

### Immunohistochemistry

Retrospectively investigated were 2 chordoid plexus tissue samples (autopsies) from 2 multiple sclerosis patients (both female, 31 and 72 years), and 15 tissue samples from patients without neurological diseases (11 men, 4 women; between 34 and 81 years, mean 60 years). The study was approved by the Ethics Committee of the University of Muenster. For histological analysis the paraffin embedded tissue samples were cut in 4 μm thick sections and stained with haematoxylin and eosin (HE). Immunohistochemistry for mouse anti-human CD3 (1:25) (Dako, Denmark) was performed using an automated immunostainer (autostainer Link48, Dako) and the avidin-biotin technique. Steamer pretreatment (citrate buffer pH6.1) for better antigen retrieval was performed.

### In Vitro Migration Assays

Primary human brain microvascular endothelial cells (HBMEC) and primary human choroid plexus epithelial cells (HCPEpiC) were purchased from ScienCell Research Laboratories (San Diego, Calif., USA). Cells were cultured on filter membrane of Transwells (3 μm pore size; Corning, N.Y., USA) for three days until reaching confluence.

Transmigration assays were performed essentially as described in Schneider-Hohendorf et al. Eur J Immunol. (2010) 40, 12, 3581-3590, incorporated herein by reference in its entirety. In case of conflict, the present specification, including definitions, will control. Briefly, PBMC in 100 μl of pre-warmed RPMI medium (RPMI, Penicilline/Streptamycine (1%), B27 supplement (2%) [Invitrogen, Darmstadt, Germany]) were added to the top of the HBMEC monolayers, and 600 μl of medium were added to the outer chamber of the inserts. The cells were allowed to migrate for six hours in a humidified cell culture incubator at 37° C. and 5% CO₂. Absolute counts of T cells were measured with Flow-Count Fluorospheres following the manufacturer's instructions (Beckman Coulter) to normalize the migration rates to standardized bead concentrations.

### Statistical Analysis

Statistical significance of differences between two groups was determined using unpaired Student's t-test except for comparisons between peripheral blood and CSF of the same patient, where the paired Student's t-test was used. Differences were considered statistically significant with p* values <0.05, with p**<0.01 and p***<0.001. Software for statistical and correlation assessment was Prism 5 (GraphPad, La Jolla, Calif., USA).

### Changes in the Composition of Major Immune Subsets Under Long-Term Natalizumab Therapy

Characterization of the major peripheral immune cell subpopulations in patients under long-term treatment with Natalizumab (n=34, treatment ≧18 months) (FIG. 2). The percentage of CD4⁺ T cells did not deviate significantly from healthy donors and untreated MS patients. The CSF compartment of these patients (n=18) was characterized by reduced percentages of B cells and CD4⁺ T cells compared to peripheral blood. The CD4/CD8 ratio in the CSF was reduced to 0.54 (11.8:21.8), indicating a stronger effect of Natalizumab on CD4 than CD8 T cells (FIG. 4).

### Impact of Long-Term Natalizumab Treatment on T-Cell Function and Phenotype

As published previously (Defer, G., et al., J Neurol Sci (2012) 314, 1-2, 138-142; Hauer, A., et al., J Neuroimmunol (2011) 234, 1-2, 148-154), treatment with Natalizumab influenced the expression of CD49d on patients' peripheral CD4⁺ T cells over time. However, it could be observed that after a decrease of surface expression to a minimum at month six of treatment, the CD49d levels surprisingly recovered. Of note, it could be shown that a patient, who developed antibodies against Natalizumab, did not downregulate CD49d on CD4⁺ T cells, which might easily be used as an early marker for the detection of patients who will not benefit from Natalizumab due to production of antibodies, as previously suggested by (Defer et al., 2012, supra). Nevertheless, CSF flow cytometry showed that CD49d levels on CD4⁺ T cells were undetectable in these patients compared to their peripheral counterparts, independent of the peripheral recovery (data not shown), whereas it has been shown repeatedly that control MS patients usually show a strongly enhanced CD49d expression on CSF T cells when compared to the periphery (data not shown and (Barrau, M. A. et al., J Neuroimmunol (2000) 111, 215-223). Additionally, CSF CD4⁺ T cells in patients under long-term treatment were characterized by missing expression of CD45RA and CCR7 (indicating an effector memory phenotype). This stands in contrast to the central-memory-like phenotype (CD45RA-CCR7⁺), which has been published previously for MS patients (Kivisäkk, P., et al., Ann Neurol (2004) 55, 627-638). Similar results were obtained for CD8⁺ T cells (data not shown). Effector memory compartments (as determined by CCR7 expression) in the periphery were not significantly affected by Natalizumab long-term therapy (data not shown) (Planas, R., et al., Eur J Immunol. (2011) doi: 10.1002/eji.201142108). CSF is generated in the choroid plexus (CP), which has also been shown in animal models to be the main entry site for leukocytes during CNS immune surveillance (Carrithers, M. D., et al., Brain (2000) 123 (Pt 6), 1092-1101) as well as inflammation (Reboldi, A., et al., Nat Immunol (2009) 10, 514-523). The inventors could show that this route is a possible entry site for T cells in the human system during homeostatic as well as pathological conditions. In both MS tissue samples as well as in 7 out of 15 controls the inventors detected CD3 positive cells in the choroid plexus. The majority of T cells was located perivascularly, however the inventors observed also single T cells in close proximity to the epithelium. As administration of Natalizumab is assumed to reduce CNS-invasion of leukocytes by inhibiting immune cell adhesion to endothelial cells of the blood-brain barrier (BBB), it was unexpected that quantitative comparison of individual migration through primary human brain-derived microvascular endothelium revealed a strong heterogeneity among Natalizumab-treated patients compared to healthy controls or untreated MS patients (FIG. 5), even though all treated patients were considered clinically stable. In contrast to this, diapedesis through primary choroid plexus-derived epithelium (simulating the blood-CSF barrier) revealed a significant and homogeneous reduction in long-term Natalizumab-treated patients (FIG. 6). As the inventors had observed a strong correlation between the expression of CD49d and treatment duration, they decided to analyze the apparent heterogeneity of transendothelial migration in relation to the months of Natalizumab treatment in more detail.

### Longitudinal Assessment of T-Cell Function Under Natalizumab Treatment: Implications for the Development of PML

Therapy-associated PML has developed as a significant challenge in a number of medical specialties over the past several your (Vinhas de Souza, M., et al., Clinical Pharmacology and Therapeutics (2012) 91, 4, 747-750). Natalizumab-associated PML has attracted considerable attention, since anti-CD49d treatment has been associated with a particularly large number of PML cases in a population, which is traditionally not at risk. Three factors have been identified that can be used as risk stratification tools. Two, namely prior immunosuppressant use and duration of therapy, are based on statistical observations, while one, presence of anti-JCV antibodies, is based on a patient's specific biologic parameter. However, even JCV seropositivity is relatively non-specific, since it simply identifies patients who have had or currently have a JCV infection and therefore the theoretical possibility of developing PML, which is JCV-mediated (Panzara et al., 2009, supra; Clifford et al., 2010, supra; Gorelik, L., et al. Annals of Neurology (2010) 68, 3, 295-303; Bloomgren et al., 2012, supra). A method to measure an individual's biological response to treatment as a way to monitor for PML risk is urgently needed. The inventors used the following groups of blood donors to differentiate between effects of MS, pre-treatments, and Natalizumab: 1) healthy controls, 2) treatment-naive MS patients, 3) MS patients before treatment with Natalizumab and 4) MS patients under long-term therapy with Natalizumab (18-66 months). The Natalizumab-treated subjects were recruited from five separate cohorts (Würzburg, Münster, Osnabrück (Germany), French Cohort Study (France) and Brascia (Italy)). In part among these five cohorts, the inventors had access to samples from 13 PML patients. Importantly, six of these patients had given blood before the diagnosis of PML (19, 26, 4, 15, 21, 20 months before PML diagnosis). As additional controls, samples from non-Natalizumab patients were analyzed who developed PML (both therapy-associated and HIV-associated; see study design and FIG. 14). Surprisingly, our results showed that the percentage of CD62L expressing cells was consistently much lower (by more than tenfold) on CD4 T cells of patients who would later on develop PML with a mean of 3.3% compared with a mean of 46.6% from non-PML Natalizumab patients (FIG. 1A, and FIG. 7 for individual dot plots).

Furthermore, samples from patients suffering from acute PML also showed a reduction or lack of CD62L expression, indicating a persistent dysregulation at least up to the point of PML diagnosis. CD62L expression showed a more diverse pattern in PML patients post diagnosis, perhaps due to the acute treatments administered for management of the PML. After PML (recovery phase, post immune reconstitution syndrome, IRIS), the percentage of CD4 cells expressing CD62L returned to a more normal range (45.4%) (FIG. 1B). Surface expression of CD62L on CD4⁺ T cells was higher than on CD8⁺ T cells. Therefore, the detection of CD62L levels on CD4⁺ T cells allowed for the most accurate discrimination of patients who eventually developed PML (data not shown). Of note, the present inventors found that using the percentage of positive cells against the isotype (in contrast to the MFI) gave the most reproducible results on different flow cytometers. The detailed gating is sketched in FIG. 7.

Expression levels of CD62L and LFA-1 were followed longitudinally in 39 Natalizumab patients in relation to transendothelial migration. Notably, levels of peripheral LFA-1 (FIG. 11) and CD62L (FIG. 12) showed a pronounced decrease within the first months, with a minimum at 6 months of therapy, followed by a subsequent gradual recovery. Functionally, this shift (reduced levels of CD49d, LFA-1, and CD62L) lead to a pronounced reduction of T-cell migration until 6 months of therapy and a subsequent recovery (FIG. 13). Between months 3 and 12 of treatment, transendothelial migration of T cells in vitro is severely reduced, which coincided with the reduced expression of CD62L and LFA-1.

Two patients in the cohort on which the data of FIG. 1D, FIG. 11, FIG. 12 and FIG. 13 are based, developed PML after 26 (FIG. 11 to FIG. 13, grey circles) and 29 (11 to FIG. 13, white circles) months of therapy. Analysis of these patients' samples (time point 0 was not available) revealed that, in contrast to the normal development, levels of LFA-1 on CD4⁺ T cells further decreased after 12 months of therapy instead of the expected recovery (FIG. 11). Additionally, CD62L expression was completely absent during the investigated time frame for one of the patient who later developed PML (FIG. 12) and the migration of T cells was already very low at month 1. Migratory function did not recover over time (FIG. 13). Notably, analysis of one of these patients more than one year after PML revealed a restored transendothelial migration/CD11a expression with poor recovery of CD62L expression.

Compared to the control patients (patients who did not develop PML), PML patients showed a lack of LFA-1 recovery, (FIG. 11), a lack or reduced of CD62L expression and a lack of CD62L recovery (FIG. 12), and reduced transendothelial migration (FIG. 13). Patients suffering from PML (n=8) associated with HIV infection or treatment with monoclonal antibodies (Natalizumab, Rituximab, Efalizumab) showed a similar lack of CD62L expression on the surface of CD4⁺ T cells at the beginning of their PML. This was again not associated with a shift towards effector memory T cells as delineated by CD45RA/CCR7 stainings (data not shown).

Perhaps importantly, the effector-memory distribution (assessed by CCR7) (Schwab et al., Multiple Sclerosis, 2012, supra; Sottini, A., et al. PLoS ONE (2012) 7, 4, e34493) of two of these patients was also altered, whereas the other four were comparable to HDs (data not shown). This may define a group with inherent risk of PML development under specific conditions.

While more research is needed, the inventors' results suggest a possible treatment paradigm where, after more than 18 Natalizumab infusions (months of therapy), the percentage of CD62L positive CD4+ cells is assessed. If the CD62L level drops below a defined threshold, which in this study could be set to approximately 25%, (FIG. 1A, dotted line in the lower right portion: defined as two times the standard deviation (SD) from the mean (m) of the control cohort (mean=46.6; SD=11.1; threshold=24.5)) an early re-assessment (e.g. one month later) of the percentage of CD62L expressing T cells may be advisable. Continuous lack of CD62L could indicate a higher risk of PML and warrant either very close clinical monitoring or a potential change in treatment regimens (Natalizumab cessation). As acute PML appears to exert variable, but not well understood effects on the immune system, CD62L should not be used as a method of PML diagnosis per se, but rather as a prospective risk factor for developing PML in the future.

Taken together, the present cell-based assay for PML risk prediction may provide an immensely valuable tool for patients and practitioners in the field of MS treatment, albeit it needs to be further validated in larger, multicenter cohorts, as well as using more patient samples collected before development of PML.

### Real Time PCR Analysis

RNA isolation was performed using Trizol® (Invitrogen, Karlsruhe, Germany) following the manufacturer's instructions. mRNA was transcribed using random hexamers and MuLV reverse transcriptase (all reagents supplied by Applied Biosystems, Foster City, USA). Gene expression assays for the detection and quantification of CD11a, Runx3 and CD62L and the housekeeping gene hS18 were purchased from Applied Biosystems and used according to the manufacturer's protocol. The Applied Biosystems Step-One Plus real-time PCR system was used, all samples were run in duplicates and each run contained several controls (healthy donor samples, wells without cDNA). There were no significant differences in cycle threshold neither within nor between the experiments. Quantification of gene expression was performed by comparing the amplification efficiencies of targets and housekeeping gene. All samples were normalized to hS18. Therefore, a lower CT value equals a higher expression of mRNA of the specific target. FIGS. 10-12 show the relative quantification of CD11a, Runx3, and CD62L as compared to hS18 on thawed PBMC from MS patients before (month 0) and in the time course of therapy (months 1, 3, 6, 12, 15-20, 21-25, 26-30, 31-40, 41-50; n=27 patients) as assessed by real-time PCR.

Long-term treatment with Natalizumab leads to changes in the peripheral immune subset distribution, which is in accordance to previous reports (increased numbers of peripheral B cells, attributed to the recruitment of precursor B cells (Krumbholz, M., et al., Neurology (2008) 71, 1350-1354) and decreased numbers of peripheral CD14⁺ monocytes (Skarica, M., et al., J Neuroimmunol (2011) 235, 1-2, 70-76). The increase in peripheral CD8⁺ T cells with no significant changes in the CD4 compartment might possibly contribute to the reversed CD4/CD8 ratio in the CSF, as observed in the cohort of these Examples and previously published (Stüve, O, et al., Arch Neurol (2006) 63, 1383-1387). Not mutually exclusive, CD4 cells might also undergo apoptosis upon encountering the antibody for a prolonged period of time, which has been published for short-term exposure in vitro (Kivisäkk, P., et al., Neurology (2009) 72, 1922-1930). The alterations in CD62L and LFA-1 expression on T cells, which have previously been shown for their CD34 stem cells (Jing, D., et al., Bone Marrow Transplantation (2010) 45, 1489-1496), might also be due to the co-localization of CD49d with CD62L on cell surface microvilli (Wedepohl, S., et al., Eur J Cell Biol. (2012) 91, 4, 257-264). In contrast to CD62L, LFA-1 is solely expressed on the planar cell body (ibid.), suggesting that the expression of LFA-1 is regulated on the gene expression level, as the connection between CD49d and LFA-1 has been shown in the inverted setting, where the blockade of CD11a increased the percentage of CD49d⁺ T cells (Harper, E. G., et al., J Invest Dermatol (2008) 128, 1173-1181).

LFA-1 and CD62L have previously been used together with CD45RA as markers to distinguish naïve, central-memory, and effector-memory T cells (Maldonado, A., et al., Arthritis Res Ther (2003) 5, R91-R96; Okumura, M., et al., J Immunol (1993) 150, 429-437). These subpopulations differ in their functional tasks with central-memory cells conferring immunity against viruses and cancer cells and effector-memory cells producing cytokines like IFN-γ and IL-4 (reviewed in (Wherry, E., et al., Nat Immunol. (2003) 4, 3, 225-234). The CSF of MS patients has been shown to mainly consist of central memory cells (Giunti, D., et al., J Leukoc Biol (2003) 73, 584-590; Kivisäkk et al., 2004, supra). This population is known to be involved in immune-mediated CNS damage during EAE (Grewal, I. S., et al., Immunity (2001) 14, 291-302) invading the CNS via the choroid plexus (Reboldi, A., et al., Nat Immunol (2009) 10, 514-523). The CSF of patients under long-term treatment with Natalizumab, however, almost exclusively contains effector-memory-like T cells. Furthermore, transepithelial migration of long-term treated Natalizumab patients is permanently reduced while transendothelial migration recovers during long-term therapy. The choroid plexus divides blood and CSF consisting of two barriers, one endothelial barrier on the blood side and one epithelial on the CSF side (Engelhardt, B., et al., Microsc Res Tech (2001) 52, 112-129) and reviewed by (Wilson, E. H., et al., J Clin Invest (2010) 120, 1368-1379). In line with previous findings in the murine system, showing that CD49d is mandatory for adhesion to the epithelial-, but not to the endothelial barrier (Steffen, B. J., et al., Am J Pathol (1996) 148, 6, 1819-1838) of the choroid plexus, it is conceivable that Natalizumab efficiently impairs this route to the CSF, resulting in a low cell count in the CSF of patients and the clinical anti-inflammatory effects. Immune surveillance, which can be accomplished using alternative routes e.g. via the subarachnoid space or directly through the blood brain barrier (reviewed by Hickey, W. F., Semin Immunol (1999) 11, 125-137), should still be functional in patients under long-term treatment with Natalizumab, as they only require crossing an endothelial barrier. In line with this, the T cells in the CSF of Natalizumab patients do not express CD49d, indicating that these cells did not use the choroid plexus as entry site into the CNS. It was shown very recently that Th17 cells in EAE migrate into the spinal cord independently of α4 integrin, whereas Th1 cells, which are supposed to be mainly responsiblefor MS pathology, use α4 integrin for the migration into the brain (Rothhammer, V., et al., J Exp Med. (2011) 21, 208, 12, 2465-2476). The invasion of these putatively pathogenic Th1 cells would therefore be inhibited by Natalizumab.

Between months 3 and 12 of treatment, transendothelial migration of T cells in vitro is severely reduced. This coincides with a reduced expression of LFA-1 and CD62L, both being molecules imperative for endothelial migration (reviewed by (Ransohoff et al., Nat Rev Immunol. (2003) 3, 7, 569-581). Interestingly, this fits to previously published data, showing peaking JCV-, but also Epstein-Bar-, Cytomegalo- and MOBP-specific T-cell responses in the same time frame indicating that the majority of primed effector T cells are efficiently trapped in the periphery (Jilek, S., et al., Lancet Neurol. (2010) 9, 3, 264-272). As a side note, the observed modulation of LFA-1 should have major implications for T-cell function besides migration, such as formation of the immunological synapse together with CD49d, cytotoxicity and antigen-specific restimulation (Mittelbrunn, M., et al., Proc Natl Acad Sci U.S.A. (2004) 27, 101(30):11058-63; Rutigliano, J. A., et al., 2004, J. Virol. (2004) 78, 6, 3014-3023; Yarovinsky, T. O., et al., Am J Respir Cell Mol. Biol. (2003) 28, 5, 607-615). Admittedly, the applied migration paradigms can only partly reflect the in vivo situation, as especially the inflammatory milieu at stages of a possible MS relapse cannot be simulated properly in vitro to date. Nevertheless, a non-inflamed cellular barrier lacking attracting stimuli on the basolateral side most likely reflects the conditions of basic immune surveillance which we consider as more important in terms of controlling a JCV reactivation event. Furthermore, the in vitro paradigms were designed to identify individuals at risk of PML on a large scale and therefore were kept as basic as possible to enable maximum experimental reproducibility.

Five patients in the inventors' cohort developed PML. One of these patients has previously been described in a case report, mainly focusing on the immune response during PML and subsequent IRIS (Schwab, N., et al., Mult Scler. (2012) 18, 335-344). Strikingly, all 5 PML patients shared three remarkable differences to the rest of the investigated cohort: 1) reduced transendothelial migration over the complete time frame, 2) missing LFA-1 recovery after 12 months, 3) missing CD62L expression and recovery. Data from Natalizumab-associated PML patients after plasma exchange revealed that migration rates normalized after stopping the Natalizumab treatment, while CD62L expression only recovered to some extent. This might hint towards a possible pre-existing condition in some patients, possibly associated with a predisposed shift in effector/memory T-cell compartments (Schwab et al., 2012, supra). All patient samples at the beginning of the PML showed the very characteristic absence of CD62L while leaving the effector-memory percentages intact (assessed by CCR7). It should be noted that especially naive (CD45RA⁺CCR7⁺) CD4 T cells lacking the expression of CD62L do not exist in controls. CD62L might therefore be the first dynamic biomarker linking all different types of PML (antibody-associated concerning treatment with Natalizumab, Efalizumab, and Rituximab, as well as HIV-associated). Further studies need to be conducted to find out if the loss of CD62L contributes to the development of PML or whether it is not functionally associated, but rather symptomatic.

Taken together, the above data support the assumption that part of the clinical efficacy of Natalizumab is due to a selective inhibition of the T-cell trafficking route through the choroid plexus into the CNS responsible for the entry of effector cells during inflammatory events i.e. MS relapse. Absent recovery of transendothelial migration could result in impaired basic CNS immune surveillance, thereby increasing the risk for PML development. It cannot be excluded that other biomarkers might also be important in patients at enhanced risk for PML. Therefore, the inventors' hypothesis ought to be evaluated and expanded in larger cohorts. However, the inventors would suggest testing patients under long-term treatment for their capacity for transendothelial migration, their peripheral levels of LFA-1, and especially CD62L to assess basic immune competence. Patients showing compromised immune surveillance should be clinically monitored very closely.

1. A method of assessing the risk of occurrence of progressive multifocal leukoencephalopathy (PML) in a subject infected with HIV, the method comprising detecting the level of T cells expressing L-selectin (CD62L) in a sample from the subject, wherein a decreased level of CD62L expressing T cells, relative to a threshold value, indicates an increased risk of occurrence of PML.

2. The method of claim 1, wherein the threshold value is based on the level of CD62L expressing T cells in a control sample.

3. The method of claim 1, further comprising detecting the level of at least one of T cells expressing P-selectin glycoprotein ligand-1 (PSGL-1) and T cells expressing lymphocyte function-associated antigen-1 (LFA-1) in the sample, wherein a decreased level of at least one of PSGL-1 and LFA-1 expressing T cells, relative to a threshold value, indicates an increased risk of occurrence of PML.

4. (canceled)

5. The method of claim 1, wherein the subject is undergoing Highly Active Antiretroviral Therapy (HAART).

6-8. (canceled)

9. The method of claim 3, wherein the threshold value of PSGL-1 is based on the level of PSGL-1 expressing T cells in a control sample, and wherein the threshold value of LFA-1 is based on the level of LFA-1 expressing T cells in a control sample.

10. The method of claim 1, wherein detecting the level of CD62L expressing T cells comprises detecting at least one of:
(i) the number of T cells in the sample from the subject that have CD62L on the cell surface,
(ii) the amount of CD62L present on T cells of the sample from the subject, and
(iii) the amount of nucleic acid formation from the SELL gene encoding CD62L in T cells of the sample from the subject.

11. The method of claim 10, wherein (i) detecting the number of T cells in the sample that have CD62L on the cell surface or (ii) detecting the amount of CD62L present on T cells of the sample comprises contacting T cells in/of the sample with a binding partner, wherein the binding partner is specific for CD62L, and detecting the amount of the binding partner binding to CD62L.

12-13. (canceled)

14. The method of claim 1, wherein the method further comprises determining the migration of CD45⁺CD49d⁺ immune cells.

15. The method of claim 14, wherein the immune cells are T cells.

16-21. (canceled)

22. A method of stratifying a subject having HIV infection, the method comprising detecting the level of T cells expressing CD62L and, optionally, at least one of PSGL-1 and LFA-1 in a sample from the subject.

23. The method of claim 22, wherein the subject is undergoing Highly Active Antiretroviral Therapy (HAART).

24. The method of claim 22, wherein the threshold value of CD62L is based on the level of CD62L expressing T cells in a control sample, wherein the threshold value of PSGL-1 is based on the level of PSGL-1 expressing T cells in a control sample, and wherein the threshold value of LFA-1 is based on the level of LFA-1 expressing T cells in a control sample.

25. The method of claim 22, wherein detecting the level of CD62L expressing T cells comprises detecting at least one of:
(i) the number of T cells in the sample from the subject that have CD62L on the cell surface,
(ii) the amount of CD62L present on T cells of the sample from the subject, and
(iii) the amount of nucleic acid formation from the SELL gene encoding CD62L in T cells of the sample from the subject,
wherein detecting the level of PSGL-1 expressing T cells comprises detecting at least one of:
(i) the number of T cells in the sample from the subject that have PSGL-1 on the cell surface,
(ii) the amount of PSGL-1 present on T cells of the sample from the subject, and
(iii) the amount of nucleic acid formation from the SELPLG gene encoding PSGL-1 in T cells of the sample from the subject,
wherein detecting the level of LFA-1 expressing T cells comprises detecting at least one of:
(i) the number of T cells in the sample from the subject that have LFA-1 on the cell surface,
(ii) the amount of LFA-1 present on T cells of the sample from the subject, and
(iii) the amount of nucleic acid formation from the ITGAL gene encoding CD11A and the ITGB2 gene encoding CD18 in T cells of the sample from the subject.

26. The method of claim 25, wherein (i) detecting the number of T cells in the sample that have CD62L, LFA-1 or PSGL-1 on the cell surface or (ii) detecting the amount of CD62L, LFA-1 or PSGL-1 present on T cells of the sample comprises contacting T cells in/of the sample with a binding partner, the binding partner being specific for CD62L, LFA-1 or PSGL-1, and detecting the amount of the binding partner binding to CD62L, LFA-1 or PSGL-1.

27. The method of claim 22, comprising repeatedly detecting the level of at least one of CD62L expressing T cells, LFA-1 expressing T cells and PSGL-1 expressing T cells in a sample from the subject.

28. The method of claim 22, wherein detecting the number of T cells in the sample from the subject that have CD62L on the cell surface comprises determining the number of T cells in the sample that do not have CD62L on the cell surface.

29. The method of claim 22, wherein the sample is one of a blood sample, a blood cell sample, a lymph sample and a sample of cerebrospinal fluid.

30. The method of claim 22, wherein the T cells are CD3⁺ T cells.

31. The method of claim 22, wherein the T cells are at least one of CD4⁺ T cells and CD8⁺ T cells.

32-84. (canceled)

85. A kit comprising a first container that includes an immunoglobulin or a proteinaceous binding molecule with immunoglobulin-like functions specific for CD62L, a second container that includes an immunoglobulin or a proteinaceous binding molecule with immunoglobulin-like functions specific for CD3, and a third container that includes an immunoglobulin or a proteinaceous binding molecule with immunoglobulin-like functions specific for at least one of CD4 and CD8.
